(19) United States
(12) Patent Application Publication
(10) Pub. No.: US 2016/0135741 A1
(43) Pub. Date: May 19, 2016

(54) APPARATUSES FOR HOME USE IN DETERMINING TISSUE WETNESS

(71) Applicant: INTERSECTION MEDICAL, INC., Carlsbad,, CA (US)

(72) Inventors: Scott Matthew CHETHAM, Del Mar, CA (US); Paul J. ERLINGER, San Clement, CA (US); Alfonso L. DE LIMON, Encinitas, CA (US); Eniko SRIVASTAVA, San Diego, CA (US)

(21) Appl. No.: 14/900,881

(22) PCT Filed: Jul. 1, 2014
(86) PCT No.: PCT/US14/45159

§ 371 Date: Dec. 22, 2015

Related U.S. Application Data
(60) Provisional application No. 61/841,900, filed on Jul. 1, 2013.
(60) Provisional application No. 61/861,360, filed on Aug. 1, 2013.

(51) Int. Cl. A61B 5/00; A61B 5/053

(52) U.S. Cl. CPC A61B 5/4878; A61B 5/0537; A61B 5/6833; A61B 2562/164; A61B 2562/0209; A61B 2562/043; A61B 2560/0425

(57) ABSTRACT

Compact and lightweight, non-invasive apparatuses to determine tissue wetness/hydration based on the frequency responses of regions of the tissue below a sensor of the apparatus. Described herein are compact and lightweight apparatuses having a sensor with an array of electrodes that is directly connected or connectable to control circuitry to attach to the back of the sensor, which can be worn by a patient. The control circuitry may include a multiplexer (MUX) coordinating the reciprocal selection of drive and sensing electrodes, and a one or more constant current sources. Methods of using these devices to detect tissue wetness are also described.

### CROSS REFERENCE TO RELATED APPLICATIONS

This patent application claims priority to U.S. provisional patent application No. 61/841,900, titled “COMPACT AND WEARABLE APPARATUSES AND METHODS FOR DETERMINING THE RELATIVE SPATIAL CHANGE IN SUBSURFACE RESISTIVITIES ACROSS FREQUENCIES IN TISSUE,” filed Jul. 1, 2013, and U.S. provisional patent application No. 61/861,360, titled “COMPACT AND WEARABLE APPARATUSES FOR HOME USE IN DETERMINING TISSUE WETNESS,” and filed Aug. 1, 2013, each of which is herein incorporated by reference in its entirety.

This patent application may be related to U.S. patent application Ser. No. 13/715,788, titled “METHODS FOR DETERMINING THE RELATIVE SPATIAL CHANGE IN SUBSURFACE RESISTIVITIES ACROSS FREQUENCIES IN TISSUE,” filed on Dec. 14, 2012, herein incorporated by reference in its entirety.

### INCORPORATION BY REFERENCE

All publications, including patents and patent applications, mentioned in this specification are herein incorporated by reference in their entirety to the same extent as if each individual publication or patent application was specifically and individually indicated to be incorporated by reference.

### FIELD

The inventions described herein relate to methods and apparatuses (devices and systems) for non-invasively determining tissue wetness based on the effects of changing current frequency on an electrical property such as resistivities (e.g., “frequency responses”) of regions of the tissue beneath an array of electrodes placed on the surface of a body. In particular, described herein are methods, devices and systems for determining tissue wetness, and particularly lung wetness, using a compact and lightweight (e.g., wearable) system, and including flexible sensors having an arrays of electrodes controlled by control module including one or more constant current sources that can reciprocally drive and sense from combinations of electrodes in the sensor.

### BACKGROUND

Tissue water content is an important and informative diagnostic parameter. Dehydration decreases cognitive and physical work capabilities, while the excessive hydration (swelling, edema) is a common symptom of cardiac, hepatic or renal pathology, malnutrition and many other pathologies and diseases. Edema causes muscle aches and pains and may affect the brain, causing headaches and irritability. Edema is a major symptom for deep venous thrombosis. It may be caused by allergies or more serious disorders of the kidney, bladder, heart, and liver, as well as food intolerance, poor diet (high sugar & salt intake), pregnancy, abuse of laxatives, diuretics, drugs, the use of contraceptive pills, hormone replacement therapy, phlebitis, etc.

For example, muscle water content (MWC) is a clinically useful measure of health. Monitoring of muscle water content can serve as an important indicator of body hydration status in athletes during the training as well as in soldiers during deployment. It is generally known that body hypohydration causes severe complications, health and performance problems, and that increasing body water weight loss causes increasing problems: water weight loss of up to 1% causes thirst, 2% may cause vague discomfort and oppression, 4% may cause increased effort for physical work, 5% may cause difficulty concentrating, 6% may cause impairment in exercise temperature regulation, increases in pulse and respiratory rate; 10% may cause spastic muscles; and 15% may cause death. Soldiers commonly dehydrate 2%-5% of body weight due to high rate of water loss from environmental exposure and performing stressful physical work. Dehydration by modest amounts (2%) decreases cognitive and physical work capabilities, while larger water losses have devastating effects on performance and health. Numerous pathologic signs and symptoms due to body dehydration include digestion problems, high blood pressure, muscle cramps, etc. MWC monitoring by an objective instrument may help prevent hazard thresholds. This is important because subjective indicators like thirst can be inadequate.

Control of MWC in athletes and soldiers could help in monitoring total body hydration during long-term endurance exercise or performance in hot weather conditions. In addition, tissue wetness may be particularly helpful in assessing lung wetness, which may be an important metric for treating cardiac disorders such as congestive heart failure.

Congestive heart failure (CHF) causes difficulty breathing because oxygen exchange in the lung is impeded by pulmonary congestion. The vast majority of CHF hospital admissions are because of difficulty breathing. Further, the high rate of CHF readmission (by some estimates approximately 24% within 30 days) is due to re-accumulation or inadequate removal of pulmonary congestion resulting in difficulty breathing. Currently, there is no quantifiable method or metric to identify pulmonary congestion and better prevent difficulty breathing and hospital admission. This problem is growing. In 2010, there was an estimated of 5.8 million CHF cases in the US, with over 670,000 new cases each year.

A subject suffering from CHF may be diagnosed using a physical exam and various imaging techniques to image the subject's chest. Treatment typically includes the use of vasodilators (e.g., ACEI/ARB), beta blockers, and diuretic therapy (e.g., Lasix). Management of treatment often proves difficult and unsuccessful. In particular, diuretic therapy is difficult for subjects and physicians to optimally manage. For example, changes in diet may require frequent changes in the diuretic therapy. Overuse (an underuse) of diuretic therapy may negatively impact clinical outcomes.

Pulmonary congestion is typically the result of high pulmonary blood pressures that drive fluid into the extra-vascular “spongy” interstitial lung tissue. FIG. 1A illustrates a pair of lungs, and shows the interstitial lung space surrounding the alveoli. High pulmonary blood pressures are present in subjects with elevated intravascular filling pressures as a result of heart failure. This high pulmonary blood pressure may also lead to increased amounts of fluid entering the extravascular space. Congestion within the extra-vascular interstitial lung tissue may prevent gas exchange ultimately, leading to a difficulty breathing that may require hospitalization. Hospital therapies are typically directed at reducing the pulmonary blood pressure by removing intravascular fluid with diuretic therapy. Although subject symptoms may improve, significant extravascular interstitial fluid may still be present. Subjects may feel well enough for discharge, but only a small change in pulmonary blood pressures will cause fluid to quickly re-accumulate, requiring readmission. Thus, subject symptoms do not reflect adequate treatment for the extent of the disease. Therefore, there is a need to detect and monitor extravascular interstitial fluid (e.g., lung wetness) and to provide an index or measure of the level extravascular interstitial fluid both instantaneously, and over time.

There are several methods for assessing total body water, as the most prominent indicator of hydration status, including methods based on bioelectrical impedance and conductance. For example, U.S. Pat. No. 4,008,712 to Nyboer discloses method and apparatus for performing electrical measurement of body electrical impedances to determine changes in total body water in normal and deranged states of the body, U.S. Pat. No. 5,615,689 to Kotler discloses a method of predicting body cell mass using impedance analysis, U.S. Pat. No. 6,280,396 to Clark discloses an apparatus and method for measuring subject's total body water content by measuring the impedance of the body, and U.S. Pat. No. 6,459,930 to Takehara et al. discloses a dehydration condition judging apparatus by measuring bioelectric impedance. However, these methods and systems have proven unreliable and difficult to implement. The aqueous tissues of the body, due to their dissolved electrolytes, are the major conductors of an electrical current, whereas body fat and bone have relatively poor conductance properties. Significant technical problems have hampered many such electrical methods for in vivo body composition analyses; impedance spectroscopy is an attempt to refine bio-impedance measurements, which measures resistance and reactance over a wide range of frequencies. A technique based on this approach is described in U.S. Pat. No. 6,125,297 to Siconolfi which describes a method and apparatus for determining volumes of body fluids in a subject using bioelectrical response spectroscopy.

Although various systems for using electrical energy have been proposed and developed, many of these systems are complex and difficult and expensive to implement. For example, systems such as electrical impedance imaging/tomography (EII/EIT) and applied potential tomography have been described elsewhere. For example, a system such as the one described in US 2007/0246046 to Teschner et al. (and others owned by the Draeger corporation) uses an electrical impedance tomography (EIT) method for reconstituting impedance distributions. In such systems, a plurality of electrodes may be arranged for this purpose on the conductive surface of the body being examined, and a control unit, usually a digital signal processor, typically ensures that a pair of (preferably) adjacent electrodes are each supplied consecutively with an electric alternating current (for example, 5 mA at 50 kHz), and the electric voltages are detected at the remaining electrodes acting as measuring electrodes and are sent to the control unit. Typically, a ring-shaped, equidistant arrangement of 16 electrodes is used, and these electrodes can be placed around the body of a subject, for example, with a belt. Alternating currents may be fed into two adjacent electrodes each, and the voltages are measured between the remaining currentless electrode pairs acting as measuring electrodes and recorded by the control unit.

Other described EIT systems, such as those illustrated in U.S. Pat. No. 7,660,617, US 2010/0228143, and WO 91/019454, do not show evidence that measurements would not vary with subject habitus, e.g., body shape or geometry.

Unfortunately, electrical impedance methods have proven difficult to reliably and accurately implement for determining tissue wetness, and particularly lung wetness. Often, additional anthropometric terms (i.e., weight, age, gender, race, shoulder width, girth, waist-to-hip ratio, and body mass index) must be included in these previous prediction models to reduce the error of the estimate within acceptable boundaries. In addition, the reliability and reproducibility of the wetness estimates may vary depending on the geometry and placement of the electrodes. Thus, current methods and systems for assessing water content based on the bioimpedance of tissues may result in low accuracy, significant dependence of testing results on the anthropometrical features of the subject and on electrolyte balance.

There is therefore a need for a simple and highly accurate method and device for monitoring tissue hydration status that can be used in a broad range of field conditions.

Described herein are systems, devices and methods that may provide an objective measure of tissue wetness. In some specific variations, the systems, devices and methods may be configured to measure pulmonary congestion (e.g., extravascular interstitial fluid) in in-subject and/or out-subject settings, including home use. For example, the systems described herein may provide non-invasive, accurate, and reproducible measures of pulmonary congestion. These systems may be referred to as lung fluid assessment monitors. Any of the systems described herein and may include executable logic to detect tissue wetness utilizing relative percent differences of apparent resistivities from the skin into the tissue derived from applying currents and measuring voltages in a specified geometric pattern of electrodes applied to the skin. The systems described herein may therefore be non-invasive, rapid, and do not use ionizing radiation.

Some variations of the systems described herein may be referred to as lung fluid assessment monitors, and may have executable logic configured to detect extravascular interstitial lung fluid utilizing determining relative spatial change in subsurface resistivities across frequencies from the skin to the lung region derived from applying currents and measuring voltages in a specified geometric pattern of electrodes applied to the skin. As mentioned, these systems may also provide an objective absolute measurement of pulmonary fluid status, such as an extravascular lung water (EVLW) index. The systems, devices and methods described herein may address many of the problems identified above, and may offer reliable and effective techniques for determining tissue wetness by determining a distribution of relative percent differences of the tissue regions beneath the electrodes to derive a value or distribution of values that are independent of the subject's body geometry. The resulting information may provide a map indicating the relative percent differences of spatial distributions of resistivities within the body across multiple frequencies. Also described herein are methods of interpreting the relative percent difference map to determine tissue wetness and, in particular, to monitor changes in tissue wetness.

### SUMMARY OF THE DISCLOSURE

Lightweight, wearable tissue wetness monitors may be particularly useful for home monitoring of subjects. However, in order to function as a home monitor, the sensor and control circuitry must be robust and easy to use. Described herein are tissue wetness, and particularly lung wetness, monitors that are well suited for use as a compact, lightweight and wearable tissue wetness monitor. Part I of this disclosure describes devices, systems and methods for non-invasively determining tissue wetness. These methods and apparatuses may determine the effect of a change in current frequency of one or more electrical property (e.g., resistivity) for sub-regions of the tissue below a sensor portion of an apparatus, such as the relative spatial change in subsurface resistivities across frequencies which may be used to determine tissue wetness for the region of tissue below the sensor.

For example, described herein are compact and lightweight devices for detecting tissue wetness. These devices may include a flexible sensor having a front and a back; an array of electrodes arranged on the front of the sensor; control circuitry configured to apply a constant current at a plurality of frequencies to the array of electrodes, the control circuitry comprising: a constant current source, a multiplexer adapted to select electrodes from the array of electrodes to act as a current source electrode, a current sink electrode, and pairs of voltage sensing electrodes, a controller connected to the multiplexer and the constant current source and adapted to drive current between different combinations of current source and current sink electrodes and to record voltages from the sensing electrodes; and a connector on the sensor adapted to connect the control circuitry to the array of electrodes.

A compact and lightweight device for detecting tissue wetness may include: a sensor having a front and a back; an array of electrodes arranged on the front of the sensor; control circuitry configured to apply a constant current at a plurality of frequencies, the control circuitry comprising: a constant current source comprising a wideband digital to analog converter, a multiplexer adapted to select electrodes from the array of electrodes to act as a current source electrode, a current sink electrode, and pairs of voltage sensing electrodes, and a controller connected to the multiplexer and the constant current source and adapted to control the multiplexer to sequentially drive current between different combinations of current source and current sink electrodes and to record sensed voltages; a connector on the sensor adapted to connect the control circuitry to the array of electrodes; and an enclosure on the back of the sensor housing the control circuitry.

A compact and lightweight device for detecting tissue wetness may include: a sensor having a front and a back; an array of electrodes arranged on the front of the sensor; control circuitry configured to apply a constant current at a plurality of frequencies, the control circuitry comprising: a first constant current source; a second constant current source that is 180 degrees out of phase with the first constant current source; a multiplexer adapted to select electrodes from the array of electrodes to act as a current source electrode, a current sink electrode, and pairs of voltage sensing electrodes, and a controller connected to the multiplexer and the first and second constant current sources, and adapted to control the multiplexer to sequentially drive current from the first constant current source on the current source electrode and current from the second constant current source on the current sink electrode and to record voltages sensed on the voltage sensing electrode pairs; a connector on the sensor adapted to connect the control circuitry to the array of electrodes; and an enclosure on the back of the sensor housing the control circuitry.

As suggested above, the constant current source may generally comprise a digital to analog converter configured as a bipolar, differential, voltage-controlled constant current source. In addition, the multiplexer may be a crosspoint switch matrix. More than one multiplexer may be used, or a multiplexer capable of selecting separate current source, current sink, first voltage sensing and second voltages sensing electrodes from an array of n electrodes (e.g., n>5, n>10, n>15, n>20, n>25, n>30). For example, the array of electrodes may comprises more than 10 electrodes, more than 15 electrodes, more than 20 electrodes, more than 25 electrodes, more than 30 electrodes, etc. The electrodes may be elongate (e.g., longer than they are wide), so that they may be positioned adjacent to each other in a line. For example, each electrode may have a length that is greater than five times its width (e.g., each electrode may be 0.8 inches long, 1 inch long, 1.2 inches long, 1.5 inches long, 1.8 inches long, 2 inches long, etc.). The electrodes may be arranged in parallel to each other down the length of the sensor.

Any of the compact, wearable apparatuses described herein may include an enclosure on the back of the sensor that houses the control circuitry. The enclosure may be removable, so that a durable/reusable housing may be used with different sensors. Thus, the sensors (electrode arrays) may be replaceable/disposable. The housing may be relatively small and lightweight (e.g., thinner than 4 inches, 3 inches, 2 inches, 1 inch, etc., above the back of the sensor).

In general, the sensor may include a bio-compatible adhesive, which may adhere both the sensor and, in some variations, the enclosure housing the control circuitry, to the patient. A battery may be included or it may be separate, and connected, e.g., via a cable, cord, etc. to the apparatus. In some variations the battery may be worn at a different location than the sensor and/or control circuitry.

In general, the control circuitry may include a second constant current source that is 180 degrees out of phase with the constant current source and wherein the controller is configured to drive current from the constant current source on the current source electrode and to drive current from the second constant current source on the current sink electrode.

Any of the apparatuses described herein may include data recording unit configured to record the voltages and an indicator of the current source electrode, a current sink electrode, and voltage sensing electrodes from which the voltage was sensed. The data recording unit may record the voltage(s) sensed as well as the identity and/or positions of the driving electrodes (e.g., current source/current sink) and sensing electrodes corresponding to the sensed voltages, as well as the frequency of the current being applied. As long as the constant current source is being used, it is not necessary to record the current. This information; the matrix of sensed voltages as well as the relative positions of the electrodes doing the driving and sensing, may be used to solve for the electrical properties within the sub-regions beneath the sensor, as described in detail below. In addition, or as an alternative to a recording unit, data may be transmitted from the device to a remote memory and/or processor for further processing. Thus, any of these device may include communications circuitry (e.g., wireless radios such as Bluetooth, etc.).

Any of these devices may also include a processor adapted to determine the effect of a change in current frequency on an electrical parameter (e.g., resistivity) of region of the tissue beneath the sensor from the data in the data recording unit, including the sensed voltages.

The control circuitry may be configured to apply a constant current at any appropriate frequencies. For example, the control circuit may be configured to apply a constant current at two or more frequencies between about 10 kHz and about 200 kHz.

As mentioned, in general these devices may be lightweight (particularly excluding the battery), so that they can be comfortably worn. For example, the devices may weigh less than 4 pounds, less than 3 pounds, less than 2 pounds, less than 1 pound, etc.

Also described herein are methods of determining tissue wetness based on the effect of a change in current frequency on an electrical parameter (e.g., frequency response) of a region of a tissue beneath a sensor, the method comprising: applying a sensor having an array of electrodes on the subject; sequentially repeating the steps of: using a multiplexer to select, from the array of electrodes, a current source electrode, a current sink electrode, and pairs of voltage sensing electrodes, applying a constant current at a plurality of different frequencies between the current source electrode and the current sink electrode and sensing voltages between the pairs of voltage sensing electrodes; and calculating an electrical parameter of the tissue beneath the sensor from the sensed voltages at the applied frequencies; and determining an indicator of tissue wetness from the electrical parameter.

In general, the multiplexer, for a given ‘round’ of sensing, may select the current source electrode the current sink electrode, and a plurality of pairs of voltage sensing electrodes from the n total electrodes in the array. The multiplexer and/or controller may apply constraints, such as avoiding “bad” electrodes, or such as only choosing voltage sensing pairs that are between the current source and current sink electrodes, etc.

Applying a constant current may comprise applying an in-phase constant current to the current source electrode and applying a 180 degree out-of-phase constant current to the current sink electrode.

In some variations the methods do not include the application step, but may include instructions to apply the sensor or a step of verifying (e.g., by checking for electrical contact with skin) that the sensor has been applied. Applying the sensor may include placing the sensor on the subject's back so that a long axis of the sensor is a proximal to distal axis that extends cranially to caudally along the subject's back, and the electrodes on the sensor may be positioned lateral to the subject's spine.

As mentioned, the device may include a processor (separately or as part of the control circuitry) to calculate from the sensed voltages and the locations of the electrodes sensing voltages/applying current one or more electrical property of sub-regions of tissue beneath the sensor at different frequencies, and may use this information to determine the effect of a change in current frequency on an electrical parameter (e.g., frequency response) for these different regions, e.g., by comparing the electrical property at the different frequencies. The frequency response generally indicates the change in the electrical property at different frequencies (e.g., as the frequency is changed). In some variation this frequency response may be compared to water (e.g., saline) and the closer it is to saline, the more “wet” the tissue region is. The tissue regions may be kept small/local (e.g., only deeper regions may be examined) or they may be averaged. In some variations, the changes in the frequency responses between the different regions may be determined (which may indicate a shift to a lower, e.g. lung, region). In any of these variations, determining the indicator of tissue wetness may comprise determining an indicator of lung wetness.

Although any electrical property may be determined, the electrical property examined may be resistivity. Other electrical properties may include resistive density, capacitance, inductance, or the like. These electrical properties may be mathematically related to each other or may be embedded in other values (e.g., inverted, offset, scaled, etc.). The method of claim 15, wherein calculating the electrical parameter comprises calculating resistivities for region of the tissue beneath the sensor. Thus, determining the indicator of tissue wetness may comprise determining the effect of a change in current frequency on an electrical parameter (frequency response) for the region of the tissue beneath the sensor, including determining the effect of a change in current frequency on an electrical parameter (e.g., resistivity) for the region of tissue beneath the sensor, and particularly the region of tissue corresponding to the lung.

Part II of this disclosure describes additional types of patch (wearable electrode arrays) configurations, and systems including such configurations. In particular, Part II also describes compact and/or wearable systems in which the electronics have been configured to fit onto the patch so that the collection electronics and the patch may be easily worn. Alternative variations of patches described may include two-dimensional arrays of electrodes, as well as patches having driving electrode positioned at a fixed distance from the sensing electrodes (e.g., an array of sensing electrodes).

Part III of this disclosure describes systems and devices incorporating techniques for optimizing the signals used to determine a relative spatial change in subsurface resistivities across frequencies. Such techniques may include filtering and/or selecting (e.g., accepting/rejecting, weighting, etc.) pairs or quads of sensing and/or driving electrodes.

In general, the effect of a change in current frequency on an electrical parameter (e.g., resistivity) of sub-regions of tissue below a sensor may be referred to as a relative spatial change in subsurface resistivities across frequencies. This may also be referred to as the relative change in resistivities between one or more frequencies for a designated spatial region below the surface on to which a sensor (or a portion of a sensor) is placed. This may also refer to the resistivity change in the region below a patch (of electrodes) relative to one or more measurements taken at different frequencies.

In general, the relative spatial change in subsurface resistivities across frequencies (RSCSRAF) refers to the relative spatial change in the region below (the subsurface region) the sensor, which may also be referred to as a patch or electrode patch. Thus, the relative spatial change may refer to the change in resistivities for subsurface regions which are located beneath the sensor at various depths (z) and lengths (x), in some cases breadth (y). The subsurface resistivities have spatial locations within the mesh elements of the subsurface (i.e. in a finite difference or finite element analysis or analytic). As described in greater detail below, the subsurface resistivities for a region under a sensor may be determined as a set of unknown resistivity variables contained within a forward problem by which each subsurface resistivity variable can be solved at each frequency at which measurements are taken, using an iterative method by minimizing the error between measured values of the system and corresponding outputs of the forward problem. This technique is called an inverse problem. Each subsurface resistivity variable determined at a given frequency can be compared against its value determined at another frequency in a relative manner in which one value is divided by the other. Examples of relative spatial change values include ratios between two values or taking a relative percent difference between the two values, at high and low frequency values, i.e.

$$\frac{{ρ}_{H}}{{ρ}_{L}} or 100 * \frac{{ρ}_{H}}{{ρ}_{L}} , 100 * \frac{{ρ}_{L} - {ρ}_{H}}{{ρ}_{L}} , or 100 ( {ρ}_{L} - {ρ}_{H} ) / {ρ}_{H} .$$

Thus, one example of relative spatial change in subsurface resistivities across frequencies the relative percent difference (RPD) in resistivity, e.g., at a low and a high frequency. As described in detail below, the relative spatial change in subsurface resistivities across frequencies can surprisingly and robustly indicate the water content (e.g., hydration) of a tissue, and may be used to determine, track, or otherwise monitor hydration status. In some variations, an index of hydration may be determined from the relative spatial change in subsurface resistivities across frequencies.

For example, a relative spatial change in subsurface resistivities across frequencies may be estimated as a relative percent difference in resistivity at a high and low frequency. In some variations, the relative percent difference of resistivity between a low and high frequency is determined for each region in the spatial distribution by multiple applied currents and measured voltages and using mathematical inversion methods to construct a spatial image of the relative percent differences in resistivity within the subsurface spatial distribution. An electrode array can be configured to consist of typically four electrodes where two electrodes are used for measuring electric current and two electrodes are used to measure differential voltage. The sensor, applied to the subject, contains numerous fixed spaced electrodes of which thousands of electrode arrays can be configured. The sensor may be referred to as a patch.

In an improvement described herein, when determining an electrical property (such as resistivity) for sub-regions of tissue beneath a sensor using the forward and reverse (inverse) methods described herein, it may be particularly useful to apply a constant current, even at the different frequencies. Thus, a constant current source capable of applying current at different frequencies would be particularly useful.

The sensors described herein may be sized and configured to allow reliable detection of the effect of a change in current frequency on an electrical parameter for subsurface regions beneath the sensor. For example, the system may be configured to provide reliable estimation of electrical properties as deep as three inches (e.g., 2″ to 2.5″) or more beneath the sensor.

The first part of this disclosure describes methods, devices and systems for determining tissue wetness. In general, the devices, methods and systems described herein may be used to determine the effect of a change in current frequency on an electrical parameter for subsurface regions beneath the sensor on a body. In some variations, a map of the effect of a change in current frequency on an electrical parameter (“frequency response” of the electrical property/properties) in sub-regions within the body may be created; this map may be displayed, or used internally and not displayed. For example, the values may be stored as a vector or matrix of values that may be used to determine or estimate a physiological parameter, such as lung wetness. In one variation, these values may be used to determine one or more hydration index, ranking or output using the non-invasive technology described herein.

In general, the system uses an applied sensor consisting of multiple electrodes of which it can be configured to apply a series of electrical currents between drive (e.g. current-injecting) electrodes of multiple frequencies and measures the voltage between selected pair of electrodes. The source of energy driving the current-applying electrodes may be a constant-current supply. In some variations, the constant current source is a bipolar, differential, voltage controlled constant current source, as described below. As mentioned, these devices, methods and systems may produce a map or matrix of the effect of a change in current frequency on (frequency response) one or more electrical parameter (such as resistivity) for sub-regions of the tissue beneath a sensor, or an index derived from the effect of a change in current frequency on an electrical parameter (such as resistivity) for sub-regions of the tissue beneath a sensor.

A map of, matrix of, or an index derived from the effect of a change in current frequency on one or more electrical parameter for sub-regions of the tissue beneath a sensor (such as the RSCSRAF) may be used by physicians to improve the medical management of subjects, including heart failure subjects, by enabling appropriate and timely interventions that reduce unnecessary hospitalizations and slows disease progression amongst a growing population of chronic heart failure subjects throughout the world.

Any of the systems, devices and methods described herein may be configured to determine a relative spatial change in subsurface resistivities across frequencies, and the resulting spatial distribution of relative changes in subsurface resistivities (relative the different frequencies examined) may be used to determine characteristics of tissue in the spatial region examined, which corresponds to the region of tissue beneath the sensor. For example, any of the systems/devices and methods described herein may be used to determine tissue water content.

Any of the systems, devices and apparatus described herein may also be configured to determine lung wetness. For example, described herein are systems for determining lung wetness including: a controller configured to control the application of currents at a plurality of different frequencies between current-injecting electrode pairs on a sensor, and to receive resulting voltage information from pairs of voltage detection electrodes on the sensor; a processing unit in communication with the controller that is configured to determine a relative spatial change in subsurface resistivities across frequencies based at least in part on parameters of the applied currents and the resulting voltages; and an output for outputting a representation of lung wetness from the relative spatial change in subsurface resistivities.

As with any of the systems and devices described herein, the systems may include a sensor or may not include a sensor but be configured for operation with a sensor. For example, a system may include a sensor comprising a plurality pairs of current-injecting electrodes and a plurality of pairs of voltage detection electrodes. In some variations, the sensor includes a plurality of four point electrode arrays each comprising one pair of current-injecting electrodes and one pair of voltage detection electrodes. The system may include a sensor comprising a plurality pairs of current-injecting electrodes and a plurality of pairs of voltage detection electrodes wherein the plurality of pairs of current-injecting electrodes and a plurality of pairs of voltage detection electrodes are arranged in a line along a tissue-contacting surface of the sensor. As mentioned above, the sensor may be configured as an adhesive patch sensor having a plurality pairs of current-injecting electrodes and a plurality of pairs of voltage detection electrodes.

In any of these systems/devices, the controller may control the application of current and the receipt of voltage to/from the subject's tissue. For example, a controller may be configured to control the application of current between the current-injecting electrode pairs at a low frequency and a high frequency, for example: a low frequency of less than about 100 kHz and a high frequency of greater than about 100 kH; a low frequency of about 50 kHz or less and a high frequency of about 200 kHz or more, etc.

The processing unit may be configured to determine the relative spatial change in subsurface resistivities across frequencies by determining a distribution of relative percent difference (RPD) in resistivities between a first applied current frequency and a second applied current frequency over a volume of the subject's tissue. The processing unit may be configured to determine the relative spatial change in subsurface resistivities across frequencies by estimating a spatial distribution of sub-surface resistivities for a region of tissue based on the applied currents and resulting voltages using an inverse problem to solve for the spatial distribution of subsurface resistivities by minimizing the error between detected values and those produced by a forward model calculating a change in subsurface resistivities. In some variations, the processor is configured to determine an index of lung wetness from the relative spatial change in subsurface resistivities across frequencies, and further wherein the output is configured to output the index of lung wetness as the representation of lung wetness.

Another example of a system for non-invasively determining lung wetness may include: a controller configured to control the application of currents at a plurality of different frequencies to pairs of current-injecting electrodes on a sensor and to concurrently receive resulting voltage information from pairs of voltage detection electrodes on the sensor; a processing unit configured to process information about the applied current and resulting voltage and to calculate apparent resistivities at different frequencies, and to determine relative percent differences of the apparent resistivities determined across different frequencies; and an output configured to present a representation of lung wetness based on the relative percent differences of the apparent resistivities determined across different frequencies. As mentioned, the controller may be configured to control the sequential application of currents at a plurality of different frequencies.

As used herein, “apparent resistivities” may include those resistivities taken or estimated at the surface of the tissue. Subsurface resistivities are typically those estimated for regions within the tissue. Generally, the resistivities referred to herein are subsurface resistivities unless the context indicates otherwise.

### SUMMARY OF PART II

As mentioned, some of the apparatus (e.g., system and device) variations described herein include a sensor, which may also be referred to as a patch. In some variations the sensor is a one-dimensional array of sensor elements (e.g., electrodes) arranged in a line. For example, a patch or sensor may include a plurality of elongate electrodes arranged in parallel and transverse to a proximal to distal axis of the sensor, wherein the electrodes comprise a plurality of voltage-sensing voltage detection electrodes and a plurality of current-applying current-injecting electrodes.

In variations including an output, the system output may be configured to provide an index of lung wetness. In some variations, the output is configured to provide a map of spatial resistivities, a map of the relative percent differences representing the region of the subject tissue beneath the electrode array, or both.

The processor may be configured to determine a slope of the spatial distribution of relative percent differences of the apparent resistivities; further wherein the output is configured to indicate that the lung is dry based on the slope. For example, the system may be configured to indicate that the lung is dry when the slope is above a threshold. The system may be configured to indicate that the lung is dry when the slope is positive.

As mentioned above, sensors for determining tissue hydration/wetness are also described. In particular, sensors for determining lung wetness are described. These sensors may be used with systems other than those explicitly described herein, though they are particularly useful for the systems/devices and methods described herein.

The configuration of the sensor may generally include a sufficient number of appropriately dimensioned (e.g., sized) stimulation/detection electrodes in a predetermined arrangement.

For example, in some variations, the sensor (and particularly a lung wetness sensor variations of the sensors generally described herein) is configured to extend cranially to caudally along an off-midline region of the subject's back while maintaining good contact along the entire region. For example, the sensor may include: a flexible support backing extending along a proximal to distal axis; a plurality of elongate electrodes arranged in parallel and transverse to the proximal to distal axis of the support backing to form an active region, wherein the active region extends between about 8 and about 14 inches along the proximal to distal axis; further wherein each of the electrodes is between about 1.5 and about 2.5 inches long and between about 0.1 and about 0.5 inches wide. This configuration may be an optimized variation for the determination of lung wetness.

From a sensor consisting of tens of electrodes, thousands of tetrapolar arrays, consisting of two current electrodes and two voltage measurement electrodes, are possible. The device is connected to the sensor from which it can select tetrapolar arrays that are advantageous in determining lung wetness. A device may use programmable logic connected to the sensor from which it can select tetrapolar array configurations and the frequency of the measurement. The tetrapolar arrays may be used by the device to determine the RSCSRAF in soft tissue. Many of the systems, devices and methods described herein are configured so that the sensor is applied to a region of the back that is just off the midline of the back, which may be particularly helpful for determining lung wetness. In some variations the sensor (or a variation of the sensor) may be applied to other body regions. For example, in some variations the sensor may be applied along the midaxillary line (e.g., a coronal line on the torso between the anterior axillary line and the posterior axillary line; the line may extend caudally from the subject's armpit). In applying the sensor, the sensor may be applied so that the tip of the sensor is as far in the subject's armpit as possible while maintaining good electrical contact. The electrodes may extend down the side of the body. In some variations, the same sensor configured as described herein for use down the off-midline region of the back may be used in midaxillary placement; in some variations a modified version of the sensor (e.g., having fewer electrodes and/or electrodes of different dimensions) may be used. The spacing of the electrodes may or may not be equally spaced. Other than the midaxillary placement, the system and method may otherwise be the same, such as determining the distribution of the RSCSRAF.

The sensor may be configured so that it extends down the subjects back without losing contact (e.g., without wrinkling, bending, buckling, or otherwise losing contact). Loss of contact over all or a portion of the sensor (and particularly the active region containing the electrodes) may result in inaccuracies in the measurements which, while they may be compensated by the other aspects of the system such as the circuitry and analysis logic, could increase the time required for the analysis or the decease the accuracy. Thus, in general, the sensor may be flexible, thin and have a small overall area yet still be large enough to take reasonable measurements. Towards this goal, the sensor may have a width that is less than 2.5 inches. In some variations, the active region extends substantially across the entire width of the flexible support backing, limiting the excess support backing region (particularly laterally from the width) which may otherwise lead to buckling or a loss of width. The support backing may comprise a polyester material and an anti-bacterial titanium oxide material (e.g., coating, etc.). Further, in some variations the sensor is conformable to the contour of a subject's back and has a thickness of less than about 5 mils.

In general, the sensor active region may include about 20 or more electrodes, about 25 or more electrodes, about 31 or more electrodes, or the like. The current-injecting electrodes may each be configured as current emitting electrodes and may be connected to a dedicated current-injecting lead for the application of current. The sensing electrodes may be configured for sensing voltage, and may each be connected to a dedicated sensing lead for sensing voltage. The leads are typically insulated connections between the surface of the electrodes applying current and/or sensing voltage and the rest of the system, including the processors and the like. In some variations the sensing electrodes and the current-injecting electrodes alternate, with the first and last electrodes in the sensor (at the proximal and distal ends, respectively) current-injecting electrodes. In some variation any electrode can be either a current drive electrode, voltage sense electrode, or both a current drive electrode and voltage sense electrode.

In some variations the sensor further comprises a proximal grip region extending proximally of the active region and a distal grip region extending distally of the active region. This grip region may be particularly useful when the electrodes extend laterally (transverse to the proximal/distal axis) across the entire width of the sensor.

In some variations the sensor further comprises a graphic print layer that may indicate how to align to anatomical landmarks.

Any appropriate conductive material may be used to form the electrodes, including silver/silver chloride. In some variations the sensor includes a conductive gel on the electrodes (or is compatible with a conductive gel). In some variations the conductive gel may include an adhesive. For example, some variations, the sensor is non-adhesive and the gel adheres it to the subject.

Any of the sensors described herein may be disposable (including single-use) or reusable. The sensors may be provided sealed and with pre-applied conductive gel. Other variations of sensor designs are described and illustrated below.

### SUMMARY OF PART III

In general, any of the apparatus (e.g., system and devices) described may be optimized so that the signals (data) provided for processing is vetted and/or filtered before being used to determine RSCSRAF. In some variations, this means that certain electrodes or pairs of (or quads of, e.g., two sensing and two driving) electrodes are rejected based on an acceptance/rejection criterion, or that raw signals are weighted based on a confidence or weighting criterion. Apparatuses and methods using such optimizing techniques may be referred to herein as “optimized” although these apparatuses may still be improved.

For example, described herein are methods and systems for the use of the optimized tetrapolar arrays and device, including the optimized placement of the sensor along an off-midline in a cranial to caudal axis along the subject's back.

In some variations, the apparatuses and methods include techniques for selecting which tetrapolar arrays within the sensor are to be used to determine tissue wetness/hydration, including lung wetness. When a tetrapolar array is used to measure apparent resistivity it is referred to as an electrical resistivity array. Different electrical resistivity arrays may provide better signals for determining tissue wetness, and the quality and sensitivity of the estimates for tissue wetness may be enhanced by using a subset of all possible arrays. In addition, not every possible electrical resistivity array need be used to determine tissue wetness.

For example, described herein are methods of determining which subset of electrical resistivity arrays from a plurality of electrodes in a sensor to use to determine a relative spatial change in subsurface resistivities across frequencies. The method may include: placing the sensor on a subject so that at least some of the electrodes are in contact with the subject's skin; scoring (e.g., ranking, rating, etc.) a plurality of electrical resistivity arrays, wherein each electrical resistivity array comprises a pair of current-injecting electrodes and a pair of voltage detection electrodes; applying current and recording voltages from a subject using only those electrical resistivity arrays meeting a selection criterion based upon their scores.

In general, scoring may comprise determining a score for an electrical resistivity array by estimating signal error for the electrodes in the electrical resistivity array and estimating a depth of investigation for the electrical resistivity array. For example, scoring may comprise determining a score for an electrical resistivity array by estimating one or more of signal error and depth of investigation for the electrical resistivity array. Scoring may comprise determining a score for one or more of error due to placement, voltage error, and current error, and depth of investigation for the electrical resistivity array. In some variations, scoring comprises combining two or more estimations to form a score for the electrical resistivity array, wherein the estimations are selected from the group consisting of: error due to placement, voltage error, and current error, and depth of investigation for the electrical resistivity array. In some variations, scoring comprises weighting each of the two or more estimations prior to combining them.

Applying current and recording voltages from a subject using only those electrical resistivity arrays meeting a selection criterion based upon their scores may include comparing scores between the electrical resistivity arrays and using only those whose scores are within a predetermined range of values. In some variations, applying current and recording voltages from a subject using only those electrical resistivity arrays meeting a selection criterion based upon their scores comprises ranking scores of the electrical resistivity arrays and using a predetermined number of the electrical resistivity arrays having the highest score values. Applying current and recording voltages from a subject using only those electrical resistivity arrays meeting a selection criterion based upon their scores may comprise ranking scores of the electrical resistivity arrays and using a predetermined number of the electrical resistivity arrays having the lowest score values.

In another variation, a method of determining which subset of electrical resistivity arrays from a plurality of electrodes in a sensor to use to determine a relative spatial change in subsurface resistivities across frequencies may include: positioning the electrodes on a subject; scoring a plurality of electrical resistivity arrays, wherein each electrical resistivity array comprises a pair of current-injecting electrodes and a pair of voltage detection electrodes, wherein the score comprises an estimation of signal error for the electrodes in the electrical resistivity array and an estimation of a depth of investigation for the electrical resistivity array; applying current and recording voltages from a subject using only those electrical resistivity arrays meeting a selection criterion based upon their scores; and determining a relative spatial change in subsurface resistivities across frequencies using applied currents and resulting voltages only from those electrical resistivity arrays meeting the selection criterion.

In general a sensor with voltage sensing/current-injecting electrodes is applied to a specific region of a subject's body and the patch configuration described above and may be optimized for use in this body region. The sensor may also include, for example, instructions, diagrams or other indicators instructing, illustrating or confirming proper placement of the sensor on the subject's body. The proper placement of electrodes on the subject's body, for example, typically includes the placement down the cranial-to-caudal axis of the subject's back, just off of the midline of the subject's back (e.g., to the right or left of the subject's spine). This configuration may allow penetration of the electrical signal within the body to a depth sufficient to reach the subject's lung region between the spine and scapula.

Also described herein is the analysis of the effect of a change in current frequency on an electrical parameter (such as resistivity) for sub-regions of the tissue beneath a sensor. The changes in effect of a change in current frequency on an electrical parameter such as resistivity in different sub-regions of the tissue beneath a sensor may be referred to as the relative spatial change in subsurface resistivities across frequencies (RSCSRAF). Data may be collected from a sensor by applying a relative technique to normalize, allowing for accurate and reliable interpretation of the RSCSRAF, including methods and systems for difference techniques to determine lung wetness as mentioned above.

Also described herein is the use of various methods to interpret the differences in effect of a change in current frequency on an electrical parameter in different sub-region of the tissue beneath the sensor to accurately diagnose, monitor, treat, track, or otherwise identify lung wetness in one or more subjects. In particular, the methods described herein (as well as system specifically adapted to preform them or enable their performance) include methods of determining if a subject is experiencing lung wetness, even in the absence of other clinical manifestations.

In general, the systems and methods described herein may implement one, or more than one, process or tests for determining lung wetness from the normalized RSCSRAF. In some variations, the methods and systems may apply sequential processes or tests to determine lung wetness.

### BRIEF DESCRIPTION OF THE DRAWINGS

FIG. 1A shows a pair of lungs, illustrating some off the structures within and surrounding them.

FIG. 1B illustrates four variations of electrical resistivity arrays that may be used.

FIG. 1C is a graph comparing 36 Wenner-Schlumberger type electrical resistivity arrays using point electrodes verse measured values using rectangular electrodes

FIG. 1D is a graph comparing 36 Wenner-Schlumberger type electrical resistivity arrays using the line-charge electrode model verses the measured values using rectangular electrodes (to be contrasted with FIG. 1C).

FIG. 1E is a representation of a saline tank model that may be used to test many of the devices, systems and methods described herein.

FIG. 1F is a table for a Wenner array of electrodes, modeled from preliminary data showing that values can be predicted to within a reasonable measurement error.

FIG. 2A illustrates a possible location for placement of electrodes or a sensor on a subject's back.

FIG. 2B shows the relationship between the scapula and lungs.

FIG. 2C is one representation of a system for measuring lung wetness as described herein.

FIG. 3A shows one example of an array of electrodes as described herein; FIG. 3B is a schematic of another variation of an array of electrodes as described herein.

FIG. 3C shows the frequency response of the resistivity of a test object (e.g., potato).

FIGS. 3D, 3E and 3F show heat maps of the RSCSRAF for the test object (potato) in a left, middle and right position within a test tank of saline.

FIG. 3G shows another exemplary heat map showing the RSCSRAF of another biological model (“ribs” formed of a potato and plastic).

FIG. 4 is a schematic of one variation of a system as described herein.

FIG. 5 is one example of a method of determining the spatial relationship of resistivities of a region of a body beneath an electrode array using the RSCSRAF.

FIG. 6A shows a spatial map of resistivities in the control case, using a tank of saline. FIG. 6B shows a spatial map of RPD in the control case, using a tank of saline. FIGS. 7A and 7B shows a spatial map of resistivities and RPD, respectively, of a test case using a test object held in a saline tank. FIGS. 8A and 8B illustrate calculated apparent resistivity (FIG. 8A) in a pseudo-section beneath the electrode array used in a test tank containing a test object. This data is generated and used to solve the forward and inverse problem to generate a spatial map of resistivity as shown in FIG. 8C and the graphical representation of apparent resistivities illustrated in FIG. 8B. The resistivity map (in FIG. 8C) may be combined with a second map at a different frequency to produce a RPD map.

FIGS. 9A and 9B illustrate another calculated apparent resistivity (FIG. 9A) in a pseudo-section beneath the electrode array and RPD. The test object include the same test object of FIGS. 8A-8B but separated from the electrode array by a second (organic) test material. FIG. 9B shows a spatial map of the apparent resistivities. FIG. 9C shows a resistivity map similar to the map shown in FIG. 8C. FIG. 10 illustrates the frequency response of water (top trace) compared to a biological material (bottom trace) from low to high frequencies of applied current.

FIG. 11 illustrates a theoretical model showing the distribution of equipotential lines and current paths for an exemplary patch electrode on a surface having varying resistivities with depth. The amount of current used to make the measurements is imperceptible to the subject/patient.

FIG. 12 schematically illustrates one variation of a method for applying a drive current and detecting (sensing) voltages to detect resistivities at different depths. In this example there are approximately 100 current drive pairs (current-injecting) and 250 voltage sensing measurements. By combining shallow and deep sensitivity measurements we can infer depth of objects. Shallow sensitivity may be achieved when voltages are measured close to the current drive pairs. Deep sensitivity may be achieved when voltages are measured away from the current drive electrodes. Approximately 100 current-injecting pairs and 250 voltage sensing measurements are used in this example. By combining shallow and deep sensitivity measurements, the depth of objects can be inferred.

FIG. 13 schematically illustrates determining a spatial distribution of resistivities for subsurface regions beneath an array of electrodes (“imaging subsurface”). Knowing the resistivity of the subsurface allows the calculation of the voltages on the surface. An image may be produced by optimizing the resistivity in the subsurface to match the voltage measurements at the surface. The volume beneath the sensor may be divided up into various subsurface layers/volumes and represented by the mesh elements (grid) beneath the electrodes in the sensor.

FIG. 14 illustrates one method of normalizing the resistivities determine as illustrated in FIG. 13 by determining a relative percent difference.

FIG. 15 is a schematic and resulting spatial distribution for an exemplary system determining the relative percent difference of a test biological material in a saline environment (biological inclusion in a saline tank). Saline has a low relative percent difference in resistivity between two frequencies 1501. An inclusion with cellular structure has a high relative percent difference in resistivity between two frequencies 1505.

FIG. 16A shows one example of a spatial distribution of relative percent differences of resistivities between a low frequency (e.g., 20 kHz) and high frequency (e.g., 200 kHz) resistivity determination for a region of a healthy subject beneath a patch electrode array.

FIG. 16B shows an example of a spatial distribution of relative percent differences similar to that shown in FIG. 16A, only taken from an edematous subject.

FIGS. 17A and 17B illustrate various methods of determining if a lung is wet or dry using a spatial distribution of relative percent differences (a subset of the relative spatial change in subsurface resistivities across frequencies) such as those shown in FIGS. 16A and 16B.

FIG. 18A illustrates another example of a spatial distribution of relative percent differences from a subject that does not have wet lung (showing a central region and gradient method of extracting information from the spatial distribution of relative subsurface resistivities). FIG. 18B illustrates on test for determining if the lung is wet or dry using the exemplary data shown in FIG. 18A.

FIG. 19 illustrates 2 metrics that provide examples of methods for determining if a lung is wet or dry applied to screened healthy subjects (circles) with dry lungs, and edematous subjects (squares) having wet lungs, using an average of a central region of the spatial distribution of relative percent differences for each subject.

FIG. 20 shows a measure of clinical progression for four subjects monitored as described herein using an average of a central region of the spatial distribution of relative percent differences for each subject.

FIG. 21 illustrates a single pole-pole array.

FIGS. 22A-22J show Table 2, showing DCIs that satisfy: (1) Relative % depth variance less than 3%; and (2) line charge K-factor does not vary from point charge K-factor by more than 3%.

FIG. 23 illustrates a graph of the distribution of the number of sub-arrays versus the depth of investigation (DOI) for sub-arrays above the threshold of deviation of the depth of investigation (Δm/m<20%) and threshold of voltage drop (ΔV>5 mV).

FIG. 24 illustrates one variation of a method of determining which arrays of electrodes to use from a sensor.

FIG. 25 shows a schematic for a one variation of drive/read electronics that can be miniaturize and configured to reside on the patch containing the electrodes.

FIG. 26 is a schematic circuit diagram for one variation of a constant current supply.

FIG. 27 is an example of another variation of a patch comprising an array of electrodes that may be used with the devices described herein.

FIGS. 28A and 28B show front and side views, respectively of another variation of an array of electrodes (patch); in this variation the patch include integrated circuitry for controlling the determination of which electrodes act as current sources and which act as sensing (voltage sensing) electrodes; the circuitry may also control the application of energy and recording of sensed voltage.

FIGS. 29A and 29B show front and side views, respectively of another variation of an array of electrodes (patch) having integrated controller circuitry, wherein the electrodes are arranged in a 2D array.

FIG. 30 is one example of a paddle-shaped sensor including an array of electrodes.

FIG. 31 is another example of a sensor comprising an array of electrodes, configured to be held by a patient/subject using the apparatus.

FIG. 32A illustrates one variation of an apparatus including a sensor connected (via over-the-shoulder cable connection) to the control circuitry. FIG. 32B is another variation with the control circuitry (with a case/housing) on the back of the sensor and connected by a cable to a battery held over the shoulder by a cable; the battery and (in some variations) case/housing may be held on an over-the shoulder strap.

FIG. 33A shows another variation of an apparatus in which the control circuitry (in a case/housing) connected to the sensor, and attached to the skin by a separate adhesive. Similarly, FIG. 33B shows a variation with a support (rather than or in addition to an adhesive) holding the electronics which is connected to the sensor (“electrode”); a power source such as a battery may be separately connected to the electronics, e.g., on the front of the patient on the support, counterbalancing the electronics.

FIG. 34A is a front view of one variation of a flexible (“semi-flexible”) sensor configured as a patch. FIG. 34B is a side view of the sensor of FIG. 34A.

FIG. 35 is one variation of a garment incorporating the sensor, showing a vest incorporating an electrode array. The control circuitry may also be included or attached.

FIG. 36A shows another variation of a garment incorporating the sensor, configured a halter or bra. Similarly, FIG. 36B shows another variation of garment incorporating the sensor, configured as a holster. The control circuitry may also be included or attached.

FIG. 37A shows a strap or band including, incorporating, or housing the sensor (patch). The control circuitry may also be included or attached.

FIG. 37B shows another variation of a strap or holster for the sensor (patch).

FIG. 38 illustrates a pre-curved sensor (patch/strip).

FIG. 39 shows an example of a sensor integrated into a chair or set.

### DETAILED DESCRIPTION

The apparatuses (e.g., devices and systems) and methods described herein allow non-invasive determination of one or more measures of soft tissue hydration which is largely indifferent to body habitus (e.g. skeletal and thoracic geometry across subjects). These methods, systems and devices use the change in an electrical parameter within a volume of tissue at different current frequencies, such as the change in resistivity at different frequencies in different sub-regions of the tissue below a sensor placed on the skin surface, to determine tissue wetness. This “frequency response” of the sub-regions of tissue may therefore indicate wetness. In particular, the change in resistivity at different frequencies in different sub-regions of the tissue will depend on the water content of the tissue. Conceptually, water (e.g., saline) has a frequency response of resistivity (a change in resistivity at different applied current frequencies) that is relatively flat, e.g., zero. Soft tissues, such as lung tissue, have a relatively higher frequency response of the electrical properties (particularly at higher frequencies). By comparing the effect of a change in current frequency on an electrical parameter (the frequency response of the applied current), an estimate of how dry or wet the tissue, and particularly the lungs, may be made. This effect of a change in current frequency on an electrical parameter may be referred to in some variations as the relative spatial change in subsurface resistivities across frequencies (RSCSRAF). The RSCSRAF may be taken within a subject to cancel out insulating boundary conditions presented by the outer shape of the subject's torso. Several metrics of soft tissue hydration may be determined from the RSCSRAF.

### Part I: Determining Relative Spatial Change in Subsurface Resistivities Across Frequencies

To provide a measure of soft tissue hydration across subjects of varying body habitus (e.g., skeletal and thoracic geometry), these systems, devices and methods compensate for the effects of varying anatomical geometry on the electrical apparent resistivity measurements. The outer shape of a subject's torso and non-conductive tissue typically presents an insulating boundary condition. Tissue structures such as bone appear as an insulator relative to muscle. It is well known that insulating boundary conditions influence the direction of the current lines of flux and thus the electric fields. The boundary has more influence on current lines and electric fields when an electrode is closer to a boundary.

For apparent resistivity measurements taken on the torso, with a an array of electrodes (the sensor) with spacing chosen to provide sensitivity to underlying tissue, the finite boundary conditions of the torso should be considered. If the finite boundary conditions are not considered or accurate, the spatial resistivity values derived by electrical resistivity tomography will be inaccurate (also known as electrical impedance tomography).

A problem arises incorporating the torso boundary shape into calculating electrical resistivity tomography, as the geometry of the human torso varies significantly among people. Models of the human torso are constructed as an attempt to incorporate the boundary into the electrical resistivity problem. A boundary model may be an ellipsoid with principle dimensions similar to that of the human torso or, the models may be imported from other imaging modalities. However, these techniques are prone to error, require external tools, and take time to measure. Whatever the model, the imperfect fit of the model to the actual subject will generate errors on the boundary of the forward problem and propagate errors to spatial resistivities found using inverse problem techniques.

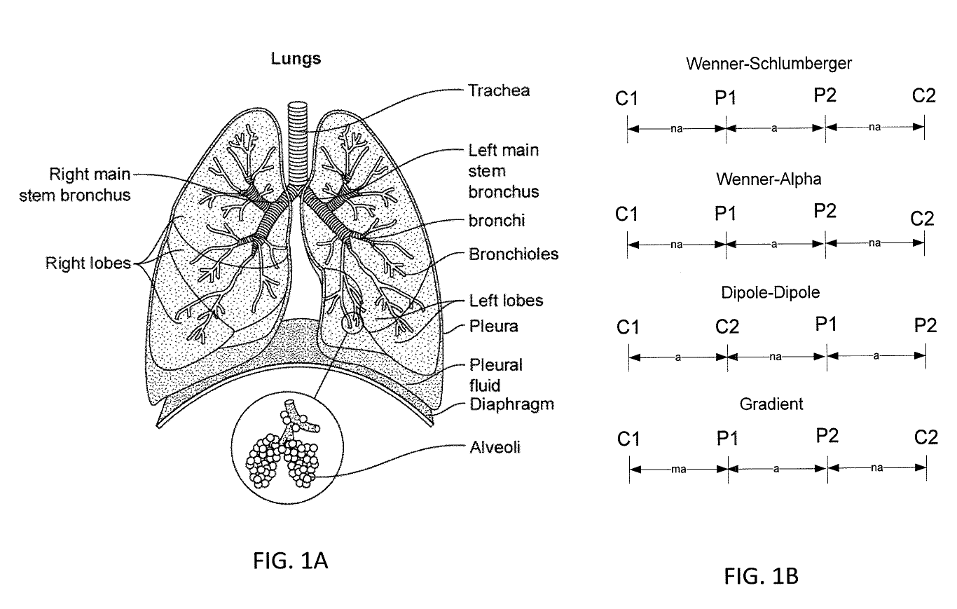

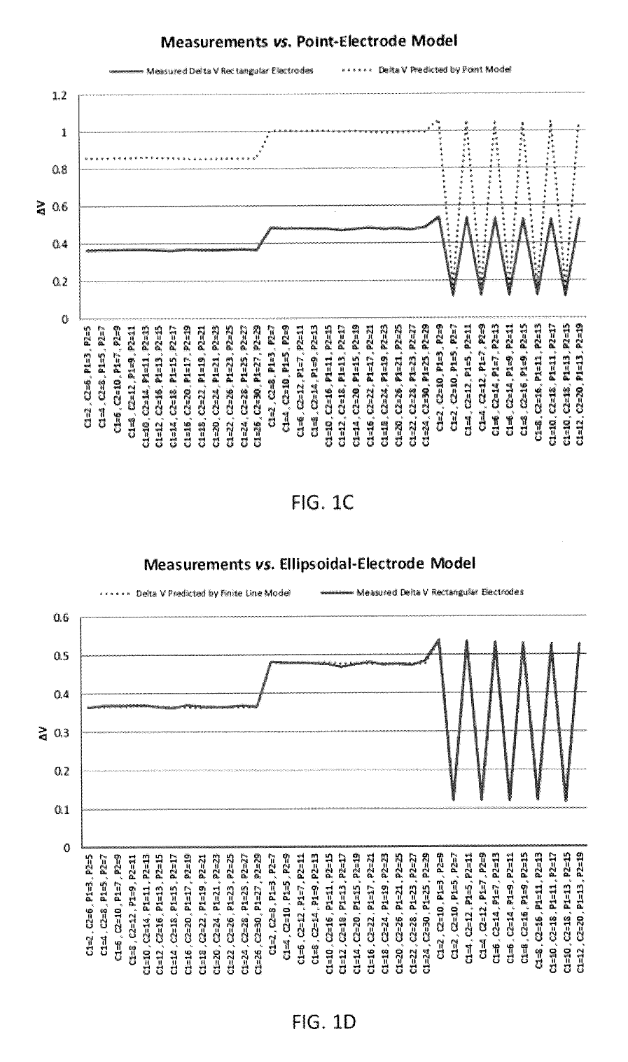

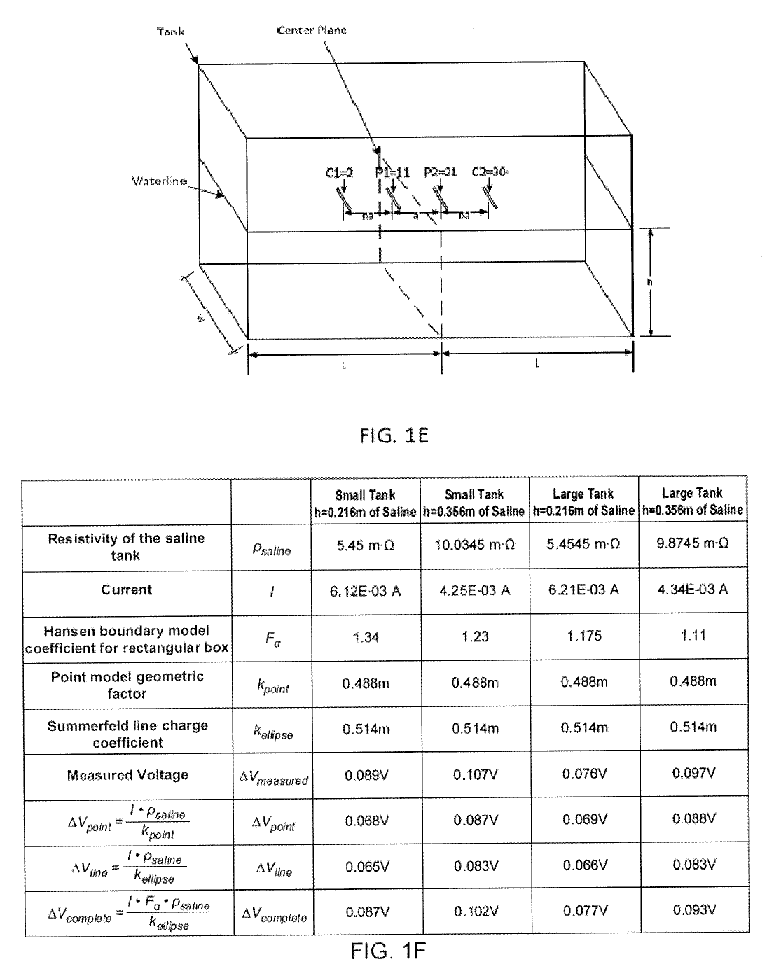

As described in greater detail below, an electrode array can be configured to consist of typically four electrodes where two electrodes are used for measuring electric current and two electrodes are used to measure differential voltage. The sensor, applied to the subject, contains numerous fixed spaced electrodes of which thousands of electrode arrays can be configured. When a tetrapolar array is used to measure apparent resistivity it is referred to as an electrical resistivity array. Different electrical resistivity arrays may provide better signals for determining tissue wetness, and the quality and sensitivity of the estimates for tissue wetness may be enhanced by using a subset of all possible arrays. The electrodes may be divided into electrical resistivity array consisting of sub-sets of the electrodes that are used for application/sensing of current/voltage to estimate tissue wetness. To understand the structure and function of an appropriate sensor (and the configuration of electrical resistivity array), consider an exemplary sensor comprising an electrical resistivity array of four electrodes that may be used for measuring electric current and differential voltage. In general, common electrical resistivity array types may include Wenner-Schlumberger, Dipole-Dipole and Gradient. Electrode arrays are widely used to measure resistivity across both large and small scales, for example, ground water reservoir surveys in geophysics and wafer fabrication applications in semiconductor manufacturing, respectively. FIG. 1B illustrates three common sub-array types, where the current is driven between C1 and C2 and voltage drop is measured across P1 and P2.

In many applications in geophysics, the electrodes may be considered as ideal points, since the electrode dimensions are significantly smaller than the electrode spacing within the array, and both the electrode dimensions and electrode spacing are significantly smaller than the size of the earth. In such a case, the

$$\frac{Δ v}{I}$$

is transformed into the apparent resistivity ρₐ by means of a geometric factor, k,

$${ρ}_{a} = k \frac{Δ V}{I} ,$$

where

$$k = \frac{2 π}{( \frac{1}{{rC}_{1} {P}_{1}} - \frac{1}{{rC}_{3} {P}_{1}} - \frac{1}{{rC}_{1} {P}_{2}} + \frac{1}{{rC}_{2} {P}_{2}} )} .$$

The equations above demonstrate that when it is appropriate to model the electrical resistivity array as points, such as in geophysics applications, the geometric factors depend on electrode spacing.

However, in practice, electrodes cannot be considered simply points, as there has to be some dimension associated with the electrode and its area has to be suitably large to inject current into the body. For measuring in a subject's body, the electrodes cannot be spaced far enough apart as to consider the electrodes as points. For example, in some variations described herein, the sensor is a combined electrical resistivity array having 31 rectangular electrodes, each electrode is approximately 2 inches long and 0.15 inches wide and spaced as close as approximately 0.36 inches, to as far as approximately 10 inches. In this case, due to the size of each electrode relative to the spacing between electrodes, it is inaccurate to model the electrodes as ideal points. Instead, each rectangular electrode may be approximated by an ellipsoidal electrode, as this electrode shape can be produced by a simple line-charge, and thus, produce a compact mathematical expression that models the voltage drop across an electrical resistivity array having electrodes with finite area.

For example, the potential at some distance r away from a point-source may decay as

$$φ ∝ \frac{1}{r} = \frac{1}{\sqrt{{x}^{2} + {y}^{2} + {z}^{2}}} ,$$

where x, y and z are the canonical coordinates in Euclidian three-space, and hence, a line charge of length 2e will produce voltage

$$φ ∝ {\int }_{- e}^{+ e} \frac{d ζ}{\sqrt{{x}^{2} + {y}^{2} + {( z - ζ )}^{2}}} = \frac{1}{e} ln \vert \frac{z + e + \sqrt{{x}^{2} + {y}^{2} + {( z + e )}^{2}}}{z - e + \sqrt{{x}^{2} + {y}^{2} + {( z - e )}^{2}}} \vert .$$

This line-charge model produces a constant voltage source on the surface of an ellipsoidal electrode with foci±e and whose semi-minor axes are equal (A. Sommerfeld, “Vorlesungenüber Theoretische Physik,” Band III: Elektrodynamik. Akademische Verlagsgesellschaft Geest and Portig, Leipzig, 4th Ed., pp. 48-49, (1967)). The analogous voltage potential is given by

$$φ ( x , y , z ) - \frac{1}{4 π e} ln \vert \frac{z + e + \sqrt{{x}^{2} + {y}^{2} + {( z + e )}^{2}}}{z - e + \sqrt{{x}^{2} + {y}^{2} + {( z - e )}^{2}}} \vert ,$$

where

$$e = \sqrt{{l}^{2} - \frac{{d}^{2}}{4}} ,$$

l is the length and d the diameter of the electrode (J. Igel, “On the Small-Scale Variability of Electrical Soil Properties and Its Influence on Geophysical Measurements,” Ph.D. Thesis, Frankfurt University, Germany (2007)). Because the electrode has geometry, the voltage contribution has to be integrated along the length l of the potential electrode,

$$φ = \frac{I ρ}{4 π e} {\int }_{0}^{l} ln \vert \frac{z + e + \sqrt{{r}^{2} + {( z + e )}^{2}}}{z - e + \sqrt{{r}^{2} + {( z - e )}^{2}}} \vert d z .$$

The geometrical factor associated with an electrical resistivity array can likewise be calculated by summing the contribution from the four electrodes,

$${K}_{ellipse} = {\int }_{0}^{l} \frac{4 π e d z}{ln \vert f ( {r}_{C 1 P 1} ) \vert - ln \vert f ( {r}_{C 1 P 2} ) \vert - ln \vert f ( {r}_{C 2 P 1} ) \vert - ln \vert f ( {r}_{C 2 P 2} ) \vert} ,$$

where

$$f ( r ) = \frac{z + e + \sqrt{{r}^{2} + {( z + e )}^{2}}}{z - e + \sqrt{{r}^{2} + {( z - e )}^{2}}} ,$$

To confirm that the ellipsoidal model better describes the voltage measurements taken using physical electrodes, a tank with a bottom area of 23.75×11.75 in² was filled to a height of 8.5 inches with saline (resistivity 5.44 Ωm), and a stainless steel version of the sensor was placed on its waterline. One hundred and five distinct drive pairs were used to inject current into the tank and this resulted in thousands of unique electrical resistivity arrays, each reporting back a single apparent resistivity. A small subset of these electrical resistivity arrays, those that are of the Wenner-Schlumberger type and those electrodes that are close to each other, are shown in FIGS. 1C and 1D. The first plot (FIG. 1C) compares the voltage drop across P1 and P2 (ΔV) as measured by the instrumentation using rectangular electrodes (solid line) and the voltage drop predicted by the point-electrode model (dotted line). As is evident from FIG. 1C, when the electrodes are close to each other, the point-electrode model fails to correctly predict the voltage drop across P1-P2.

The second ellipsoidal electrode model achieves good agreement with experimental values, as can be seen in FIG. 1D. In this case, even electrodes located next to each other (i.e., C1=2, C2=6, P1=3 and P2=5), which were mismatched by over 130% using a point-electrode model, match with an error of less than 1% using this line-electrode model when compared to measured data. This good agreement with experimental data implies that the line-electrode model correctly captures the size of the voltage drop, and hence, can be used to specify electrical resistivity arrays whose voltage drop can be ascertained accurately to some predefined voltage threshold given by the accuracy of the measurement device.

In the systems, devices and methods described herein, because of the relative spacing of electrodes with respect to the size of the human torso, the human torso cannot be modeled as an infinite half sphere, as with geophysical models. The outer shape of the torso, given the approximate electrode spacing of the sensor, will substantially influence the current lines and electric fields within the torso. Analytical models that translate tetrapolar apparent resistivity measurements on a finite bounded model are often difficult to derive. However, the literature provides several analytical models for simple geometric shapes. A case in point would be the analytical model of a tetrapolar apparent resistivity measurement on a flat surface of a bar shaped semiconductor provided by Hansen (E. Hansen, “On the influence of shape and variations in conductivity of the sample on four-point measurements,” Applied Scientific Research, Section B, Vol. 8, No. 1, pg. 93-104, (1960)), where the electrodes are modeled as points. Hansen's analytical model is derived for a bar shaped semiconductor. Hansen derives his model from point electrodes; it does not take into account electrode geometry. However, when the distance between electrodes is sufficiently large, the voltages of rectangular electrode converge to that of a point electrode. The extension of Hansen's equation to a box model is shown below

$${F}_{α} = \frac{2 π {s}^{2}}{ah} + \frac{16 π s}{ah} {\sum }_{{m = 0}_{( m , n ) \neq ( 0 , 0 )}}^{∞} {\sum }_{n = 0}^{∞} \frac{cosh β ( l - \frac{3 s}{2} ) sinh ( \frac{β s}{2} )}{( 1 + {δ}_{0 , m} ) ( 1 + {δ}_{0 , n} ) βcosh ( β l )} + \frac{16 π s}{ah} {\sum }_{m = 1}^{∞} {\sum }_{n = 0}^{∞} \frac{{( - 1 )}^{m - 1} {sin}^{2} ( m πΔ / a ) cosh γ ( l - \frac{3 s}{2} ) sinh ( \frac{γ s}{2} )}{( 1 + {δ}_{0 , n} ) γcosh ( γ l )} ,$$

where β=(2π/a)\sqrt{m²+(na/2h)²} and γ=(π/a)\sqrt{m²+(na/h)²}.

To mathematically model the forward problem of one of the electrical resistivity arrays, out of the thousands available from a sensor, it is possible to combine the translation constants of Summerfeld and Hansen in the following equation:

$$Δ {V}_{complete} = \frac{{F}_{α} {ρ}_{α} I}{{k}_{ellipsoid}}$$

To verify the correction factor, F_{α}, the voltage across P1 and P1 was predicted for a Wenner-Alpha electrical resistivity array from the sensor over three homogenous saline tank models. FIG. 1E illustrates a tank model. In this example, the stainless steel electrode dimensions are: 0.0508m×0.00381m; the tank dimensions are: L=0.301m, a=0.298m; the electrode spacing is: s=0.085m.

As shown in the table of FIG. 1F, for a Wenner resistivity array; given a current, the resistivity of the saline, the Hansen's boundary correction coefficient, Summerfeld's line charge coefficient, and the voltage drop across P1-P2 can be predicted to some measurement error. For example, a reasonable error may be error of less than 5%.

In the preceding calculations, F_{α}, kₚₒᵢₙₜ, kₑₗₗᵢₚₛₑ and ρ are real numbers. However, while F_{α}, kₚₒᵢₙₜ, kₑₗₗᵢₚₛₑ are real numbers, for capacitive biomaterials, ρ is a frequency dependent complex number

$$ρ = {ρ}^{′} - {jρ}^{″} = \frac{1}{σ} = \frac{( {σ}^{′} - {jσ}^{″} )}{{\vert σ \vert}^{2}} ,$$

where

σ≡σ′+jσ″,

∈≡∈′−j∈″≡(∈′ᵣ−j∈″ᵣ)∈₀,

with the following properties listed in the table below.

| | |
|---|---|
| σ = jωε | σ″ = ωε′ |
| σ′ = ωε″ | ${ɛ}^{″} = \frac{{σ}^{′}}{ω}$ |
| ${ɛ}^{′} = \frac{{σ}^{″}}{ω}$ | ${ρ}^{″} = \frac{{σ}^{″}}{{\vert σ \vert}^{2}}$ |
| ${ρ}^{′} - \frac{{σ}^{′}}{{\vert σ \vert}^{2}}$ | |

For rectangular electrodes on a sufficiently large box, the apparent resistivity is

$${ρ}_{α} = \frac{{k}_{ellipsoid} Δ {V}_{complete}}{{F}_{α} I} .$$

Because the resistivity is complex valued and frequency dependent, the calculated spatial resistivity at two frequencies would yield a spatial resistivity for the low frequency and for the high frequency: ρ_{α}^{low} and ρ_{α}^{high}. Note that in taking the relative percent difference (RPD which is a special case RSCSRAF) between ρ_{α}^{low} and ρ_{α}^{high}, the boundary and geometrical factors cancel, such that

$$RPD = 100 \frac{{ρ}_{α}^{low} - {ρ}_{α}^{high}}{{ρ}_{α}^{high}} ,$$

which can be simplified to

$$RPD = 100 ( \frac{{V}^{low}}{{V}^{high}} \cdot \frac{{I}^{high}}{{I}^{low}} - 1 ) ,$$

and captures the change in resistivity of the subsurface without requiring geometrical information of the sensor or boundaries.

Thus, as described, a system may be built on the realization that the RSCSASRAF captures the change in resistivity of the subsurface without requiring geometrical information of electrical resistivity array or boundaries, and to expand the concept to spatial resistivity, resistivity beneath the subsurface of a sensor.

The systems described herein may use a combined electrical resistivity array, which may be referred to as a sensor, which serves as the subject-applied portion of a medical device apparatus or system that determines the spatial relationship of the RSCSARAF in soft tissue beneath the surface of the sensor. The sensor may contain tens of fixed-spaced electrodes of which thousands of four point resistivity arrays can be configured. The medical device apparatus may determine the spatial relationship of the RSCSARAF in each cell of a mathematically determined, two or three-dimensional, multi-cell, cross-sectional grid, extending horizontally and vertically beneath the sensor. The grid may span a maximum horizontal distance equal to that of sensor and may be sized in the vertical dimension to a specified depth of investigation (DOI). The dimensional may be determined for each cell in the grid by driving current and measuring voltage in a manner that is common in electrical resistivity array surveys and using mathematical inversion methods to construct a spatial image of the dimensional within the grid. The sensor and medical device apparatus have the capability of determining soft tissue hydration.

In general, the devices and systems described herein are used by first placing the sensor (e.g., an array of electrodes including electrodes arranged in a predetermined pattern) on a subject. The placement location may be chosen to optimize the sensitivity and result of the system. Thereafter, the system may use the sensor to measure one or more electrical properties 103 (e.g., voltages, complex impedances, complex conductivities, etc.) from the subject. The system or device then typically determines the spatial relationship of resistivities (or a derived value) using the known arrangement of the electrodes in the array as well as the known applied currents and the sensed electrical properties at a plurality of the electrodes in the sensor and solving inverse problems. In general, the subsurface spatial resistivities are solved for using this information. However, because the apparent resistivities are sensitive to a geometric factor (referred to herein as k, or the k factor) that depends on the boundary conditions and arrangement of the electrodes, the systems described herein are configured and adapted to minimize or eliminate the effects of the geometric k factor. In some variations, the system therefore calculates a relative percent difference between the spatial arrangements of resistivities determined at a low frequency and at a high frequency to eliminate the geometric k factor. As described in greater detail below, this calculation of relative percent differences may allow a normalized percent difference that more accurately reflect changes in resistivity reflecting lung wetness.

The systems described herein may determine (from the applied currents and measured voltages) various data types, including particularly spatial estimates of resistivities within the volume of tissue beneath the array of electrodes, and/or relative percent differences between spatial resistivities at different (e.g., between a high and a low) frequencies.

### Resistivities

The systems described herein may use either or both apparent resistivities or the logarithm of apparent resistivities. The apparent resistivities are given by the mathematical expression:

$${ρ}_{α} = k \frac{Δ V}{I}$$

where k is a geometric factor that depends on the boundary conditions and arrangement of electrodes, ΔV is the differential voltage of interest, and I is the current passing through a region of the body between any pair of current drive electrodes. The system may provide multiple complex valued apparent resistivities for the multiple combinations of electrode drive pairs.

### Relative Percent Difference (RPD)

Relative percent difference (which, is a type of RSCSRAF and is defined as the relative percent change in the magnitudes of spatial resistivities between two separate frequencies.

$$PFE - 100 * \frac{{ρ}_{α} ( {ω}_{L} ) - {ρ}_{α} ( {ω}_{H} )}{{ρ}_{α} ( {ω}_{H} )}$$

Notice, by using the RPD, the geometric factor k cancels.

### Phase of Apparent Resistivities

The systems described herein may also measure ΔV and I in complex form so apparent resistivities can take the form:

ρₐ=k(real+imag).

The phase angle of apparent resistivity is given by:

$$θ = arctan \frac{k ( imag )}{k ( real )} .$$

Again, when describing the phase angle of the apparent resistivity, the geometric factor k cancels.

### Forward and Inverse Problems.

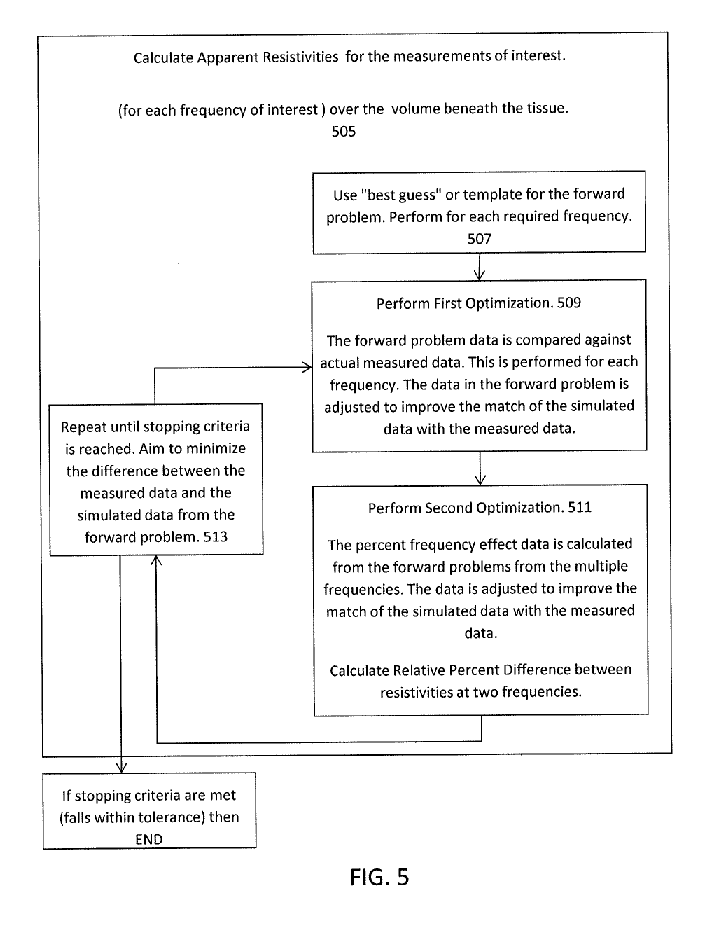

FIG. 5 illustrates one method to determine a distribution of resistivities of a subsurface below a sensor, when the sensor is applied to a human body of unknown geometry. In FIG. 5, the processor, after receiving the applied currents and sensed voltages (complex) from the sensor of a predetermined configuration first calculates the apparent resistivities for each electrical resistivity array. The subsurface resistivities may be determined for each frequency of interest using a mathematical model of the sub-surfaces 505 (e.g., solving the forward problem).

A finite-difference or finite-element method may be used to model the subsurface and tie the spatial relationships of the resistivities and properties of resistivities of the subsurface to the measured apparent resistivities and properties of apparent resistivities from the measured values from the system. A quantitative approximation of depth may be derived for the model using the Frechet derivative or sensitivity function and is used to adjust the size of the blocks within the model. The “median depth of investigation” (DOI) may be used as a robust approximation to depth. The median depth of investigation is the depth in which from the sensitivity function, the depth above the DOI, has the same influence on the measured potential as the depths below the DOI.

Once the forward problem has been initially “solved,” 507, 509 it may be optimized in conjunction with the inverse problem 511. In some variations, the methods and systems described herein run two optimization problems jointly, either consecutively or sequentially, to determine the spatial relationships of resistivities and properties of resistivities of the subsurface below a sensor on a human with unknown geometry. In this optimization problem, the initial model (forward problem result) is modified in a smoothness-constrained iterative manner so that the difference between the model response (forward problem) and the observed data values (measured values from the system) is reduced to within acceptable limits.

This inverse problem is described in terms of an example as applied to one variation of the systems and methods described. For example, the inverse problem may infer the makeup of the body given a sample of voltage measures on the body's surface for a given current injection location. Because the set of data given to the inverse problem is far more limited, calculating the subsurface electrical properties from a few surface measurements cannot be calculated directly by plugging them into an equation. Thus, a set of “guesses” of the body's internal properties may be made and using the forward problem; each of their resultant voltages may be compared to the voltage measurements taken on the subject's body (an optimization process selects how to change the guesses). The forward model whose resultant voltages most closely resemble the measured voltage is selected as the most likely representation of the subject's internal properties.

In the first optimization, multiple apparent resistivity measurements taken with the sensor at one frequency serve as the observed or measured data. Reference geometry may be used to determine the geometric factors to calculate each apparent resistivity measurement. The reference geometry may be a rectangular volume approximation of the human thorax. The geometric factors can be empirically determined by using our system to measure each (ΔV)/I for each apparent resistivity position in the array when the sensor is placed just below the waterline of a saline tank with known resistivity and volume proportions similar to that of a human torso. Each geometric factor is determined by the following equation:

$$k = \frac{I * {ρ}_{saiine}}{Δ V}$$

The first optimization runs a set of forward problems, using a smoothness constraint to determine the spatial resistivities in the model such that the error in the model response to the measured values is minimized to within acceptable limits. This provides a spatial resistivity map at one frequency; however, an error has been introduced by the mismatch of the human geometry to that of the saline tank, which will be corrected by the methods described below.

The first optimization problem runs jointly (or separately) with a second optimization problem to seek a solution to the second optimization model in terms of the RPD or phase. Thus, in some variations the relative percent difference 521 may be optimized within the process described in block 501 of FIG. 5. One or both may prove useful. There are two choices in setting up the second optimization problem; either to determine the spatial change in resistivities between two frequencies beneath the sensor or to determine the relationship of the phase of resistivities beneath the sensor. Either choice reduces the influence of the human geometry by the canceling out effect of the geometric factors.

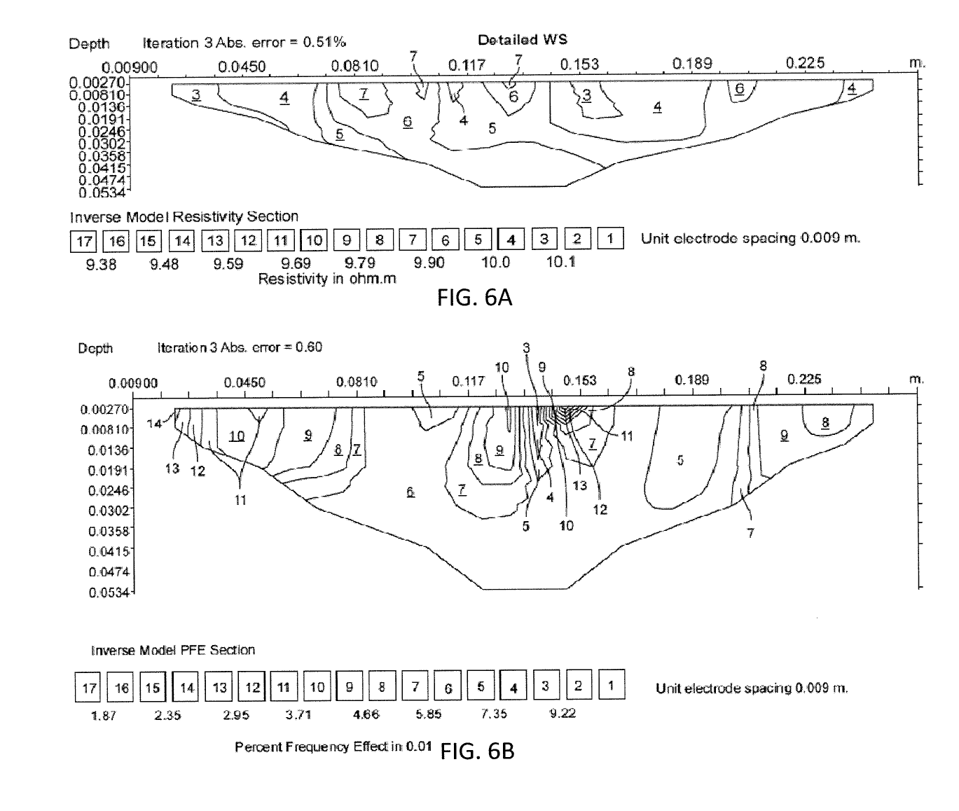

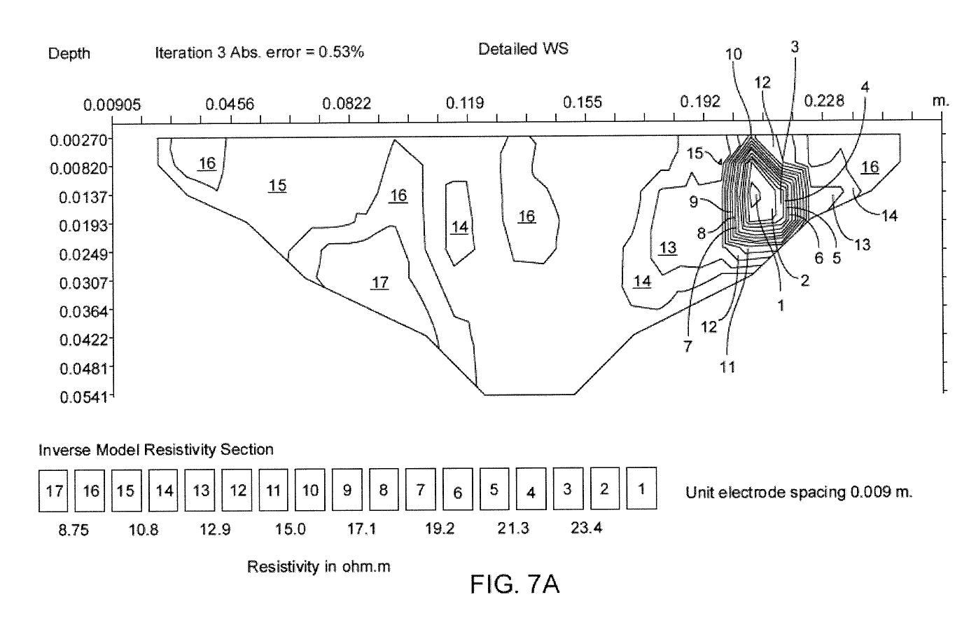

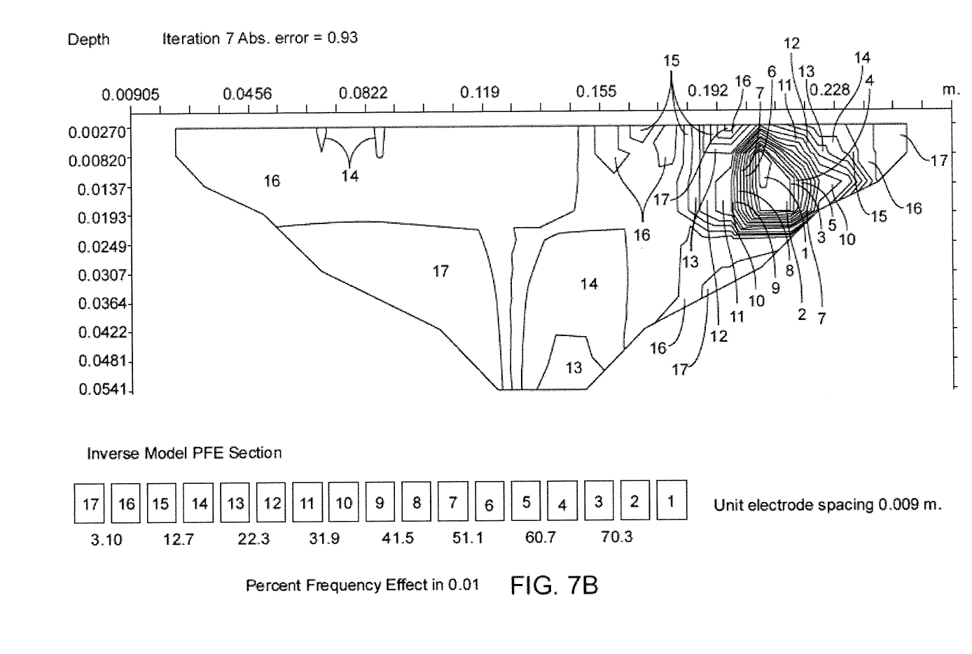

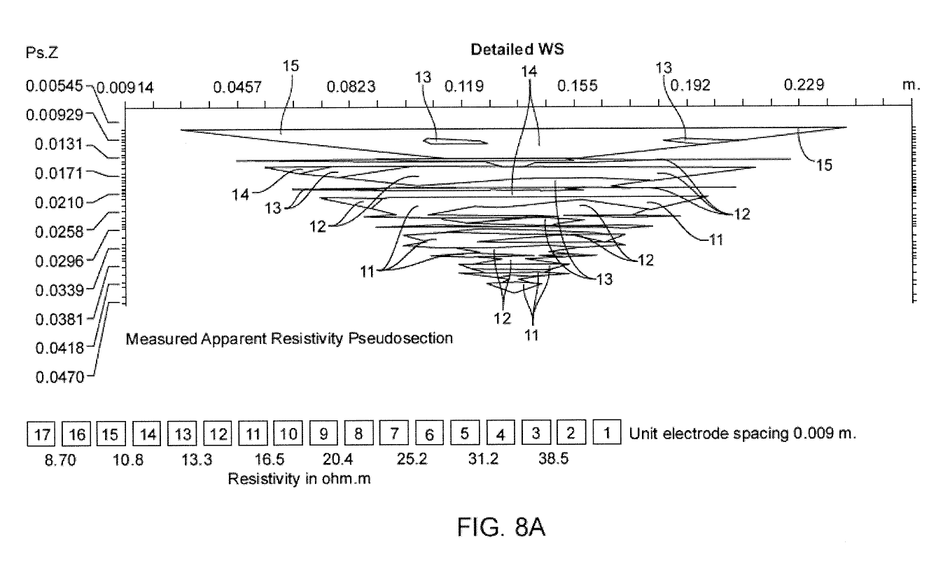

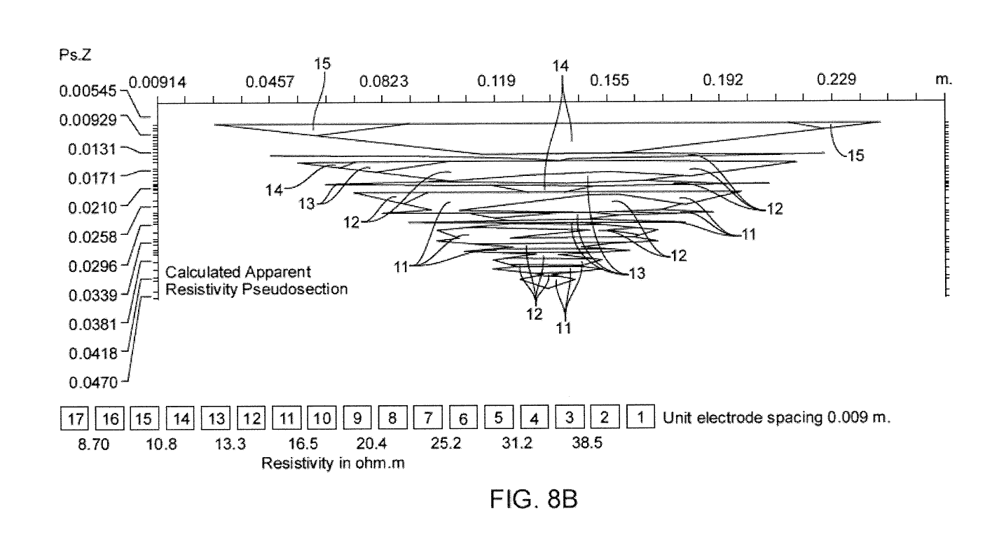

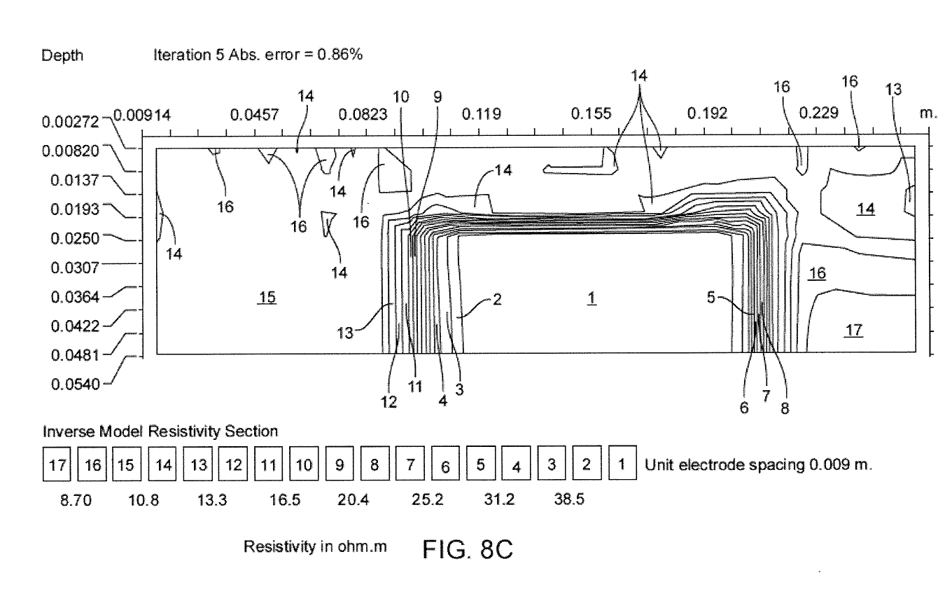

In some variations, two images are produced, which may be depicted as “heat maps” showing relative intensity values. The first heat map image may be produced by the first optimization problem and shows the spatial resistivity mapping at a single frequency in terms of reference geometry. The mismatch between the reference geometry and the human geometry propagates an error in the solution. Yet, this image may contain some useful information because the reference geometry is of the general scale as a human. An example of this may be seen in FIGS. 6A and 7A, described below. The second image is produced by the solution from the second optimization problem. The second optimization problem is mathematically linked to the first optimization problem and therefore in some variations, may not be run on its own. The two optimization problems can be run jointly or sequentially. Examples of the second image are shown in FIGS. 6B and 7B.

The value in the second image lies in that the heat map produced is the RPD of the resistivities of the subsurface which has less influence from human geometry. In one application, e.g., the detection of lung wetness, we expect edema to present in the second image as either lower RPD values or as lower phase angles in the regions of interest; whichever method for the second optimization is chosen. It may be that two second images should be generated, one of the RPD type and one of the phase type. These two images together may provide more information about the subsurface.

Thus, for example, in one embodiment, when driving from 15 drive electrodes at each of four frequencies, a total of 252 times 4 (or 1008) measurements may be recorded and processed as indicated above. The measurements may be sorted by pseudo depth resulting in a triangular shaped profile sorted by spacing of the regions. Solving for the homogenous case, when all of the regions and sub-regions have approximately the same apparent resistivity (e.g., in a saline test bath), the k factors for this arrangement can be easily calculated. This is illustrated in FIGS. 6A and 6B, which shows a tank of saline to which the sensor has been placed in contact. For example, FIG. 6A shows an image representing the inverse model resistivities through a reconstructed pseudo-section from the tank filled with saline. The image in FIG. 6A is a resistivity image generated at 50 kHz. In a perfect system the image would be completely uniform (e.g., having a resistivity of 10 Ohm meters); in the experimental result shown in FIG. 6A, the values vary from approximately 9.77 to 10.31.

FIG. 6B shows a heat map of the same pseudo-section region of FIG. 6A, showing a RPD image of the frequency response between 50 k Hz and 200 kHz. As above, in an ideal system the values would be all zeroes; as shown, the values vary between 1.22 and 10.50 with the majority of the region begin approximately 1.22.

As a proof of principle of the method illustrated above, when an object, including biological material, is positioned beneath the sensor, the system may provide the distribution of resistivities. For example, the saline tank setup used for FIGS. 6A and 6B may be used as a proof of principle by adding various materials into the tank. FIGS. 7A and 7B illustrate one variation in which the sensor is used to determine the arrangement of resistivities of a test material (a yam) added to the saline tank. In this example, a yam has been added to the upper right side of the tank beneath the sensor, as can be seen in the images of FIGS. 7A and 7B. Similar to FIGS. 6A and 6B, the upper image (FIG. 7A) is a resistivity image from data taken at 50 kHz. The yam can clearly be identified in the upper right quadrant. The lower image (FIG. 7B) shows the RPD, the change in resistivity as a function of frequency between 50 kHz and 200 kHz. The saline has a low value of approximately 1.79 or less in the image. The yam value is in the 40's.

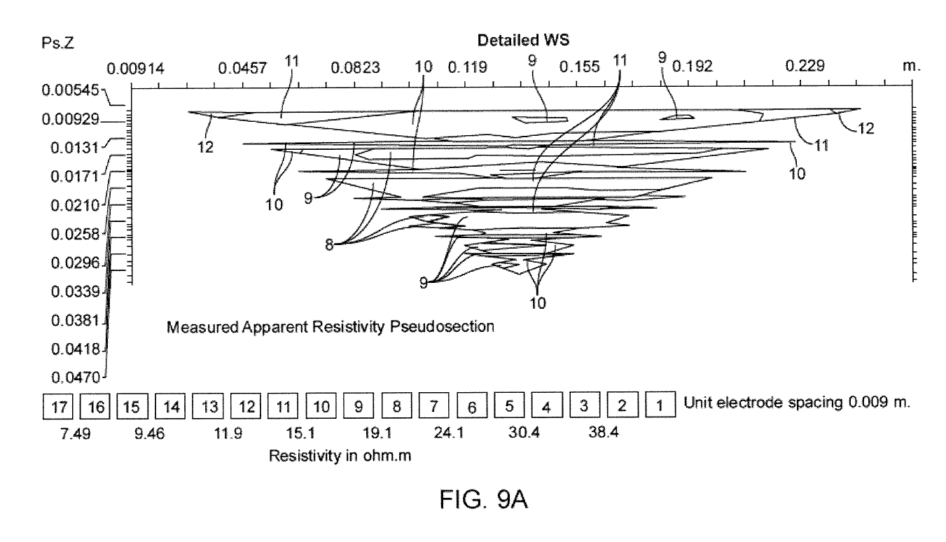

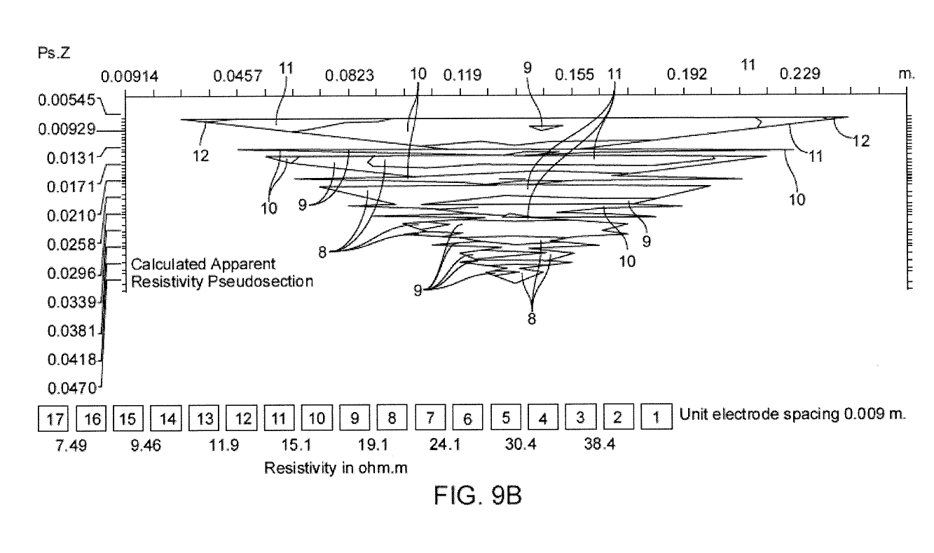

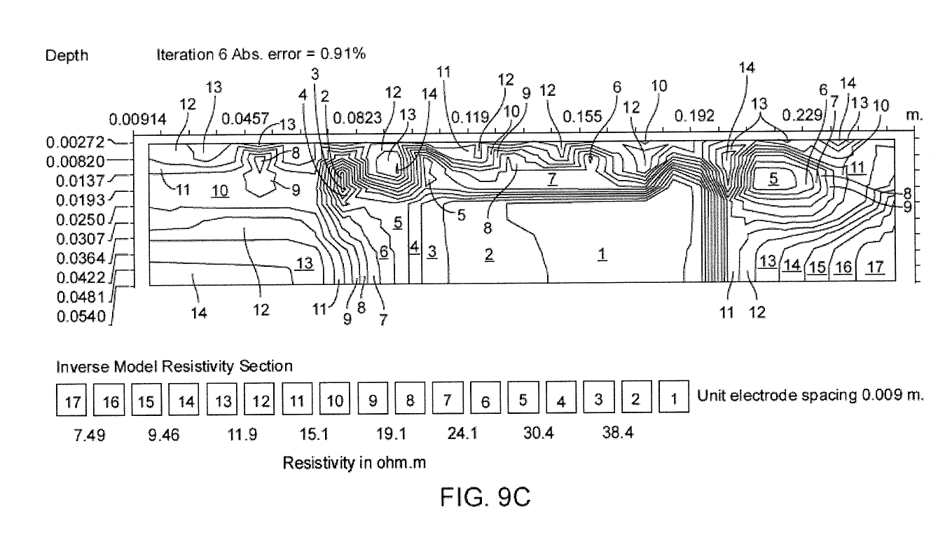

For example, taking multiple frequencies may allow the determination of high-contrast differences in resistivities. Experimentally data such as that shown in FIGS. 6A-7B illustrate that the system may be able to interpret differences between electrically conductive and insulative regions with high contrast. This is also illustrated in FIG. 8A-9B.

In determine the spacing of the electrodes for the array, which may affect the sensitivity of the resulting heat map, the spacing may be based on the sensitivity function (e.g., the Frechet derivative). The system may also change the cell (voxel) size and/or shape. In the figures illustrated here, the size/shape is assumed to be square; however other sizes and shapes may be used.

FIG. 8A illustrates apparent resistivities. In this example, the test object is a glass beaker. The data is generated and used to solve the forward and inverse problem to generate a spatial map of resistivities as illustrated in FIG. 8C.

The same set-up may be used to image through a tissue similar to the tissue found in the subject's body (e.g., skin, muscle, bone) in order to image lung tissue. FIGS. 9A and 9B illustrate another example of computed spatial resistivity. The test object is the same beaker used in FIGS. 8A-8C, but also includes a second (organic) test material near the electrode surface. In this example, the second test material is muscle and bone (ribs) corresponding to (uncooked) pork tissue. As shown in FIG. 9C, the system was able to distinguish the flask (insulative material) beneath the layer of muscle and bone, based on resistivity of the different regions. When RPD is used to examine the bath, the beaker is not visible in the image as it has no frequency response.

The system may be adapted to apply a multi-frequency current (e.g., from a drive electrode) to a subject's skin and to sense voltage (from a sensing electrode) on the subject's skin at a plurality of frequencies. For example, the system can receive a multi-frequency current signal from a DAC, as described below, and, when the relay circuit is closed, apply a multi-frequency current to a subject's skin through the sensor. A current signal may be applied simultaneously to the subject at a plurality of different frequencies, or at only one frequency at a time (sequentially). When a relay circuit is open, the electrode interface can sense or measure voltage across the subject's skin at a plurality of frequencies. Simultaneously sensing voltage at multiple frequencies can be achieved by including a plurality of correlators in the system, where each correlator corresponds to a different frequency of measurement. Each correlator can act as a narrow band filter that allows the system to extract a complex voltage at a particular frequency of measurement. For example, one embodiment includes 20 correlators in the system corresponding to 20 different frequencies of measurement. Each correlator in the system can provide, as an output, a complex voltage having an in-phase component and a quadrature component for a particular frequency of measurement. It can be appreciated that the correlators can be implemented in a number of ways, as known in the art. In practice, a system may include only one correlator, or more than one.

In some variations the sensor may include active circuitry components that eliminate or reduce capacitance on the electrode interface. For example, when a multi-frequency current is applied to the electrode surface, an input capacitance can result at the electrode surface. To neutralize this capacitance, a capacitance neutralization circuit can be implemented. The capacitance neutralization circuit can feed the input capacitance back into an input of the electrode interface to effectively eliminate the input capacitance from the interface. The operation of a relay circuit, as described above, can also add a capacitance to the electrode interface. The capacitive effect of the relay circuit can be neutralized by adding an electrostatic shield around the relay circuit and driving the electrostatic shield when the relay circuit is closed.

Thus, in some variations the sensor may include active circuitry to enhance collection and processing of data. In other variations the processing of collected and/or applied signals may be done by the controller upstream from the sensor. By placing the active circuitry near to the electrode surface, the system can apply a multi-frequency signal to a subject, sense voltage on the subject, process the voltage to extract a complex voltage at each of the measured frequencies, and output the signals for further processing and analysis. It should be appreciated that the application of current, sensing of voltage, and data processing may all be done by the active circuitry on the sensor. Placing the active circuitry near the electrode surface may also reduce the capacitance at the electrode surface and minimizing the amount of any additional noise added to the applied and measured signals. Thus, the system of the present invention may eliminate excess capacitance and noise allows accurate measurement of the biological parameters of a subject, instead of measuring the effects of the electrode interface itself and noise.

Water, as opposed to most biological materials such as tissue, can be modeled electrically as nearly purely resistive, and has a relatively flat frequency response, as shown in the top of FIG. 10. By comparison, biological tissue has a dynamic frequency response, and may be modeled as an RC circuit, as shown in the bottom of FIG. 10. In FIG. 10, it is generally true that as frequency increases from low (e.g., <100 Hz) to high (e.g., <100 Hz), the frequency response of biological tissue increases, while the frequency response of water remains constant. Thus, it may be expected that when looking at lung wetness, the more that the frequency response of the sampled region behaves like water (e.g., has a relatively flatter frequency response) the more wetter that the region may be. Although this is a very simplified model, the use of the relative percent difference may take advantage of this simple relationship by comparing the apparent resistivities at low frequency versus high frequency. Wetter tissues may be expected to have a flatter frequency response, which may be seen by examining the relative percent differences between apparent resistivities at low versus high test frequencies.

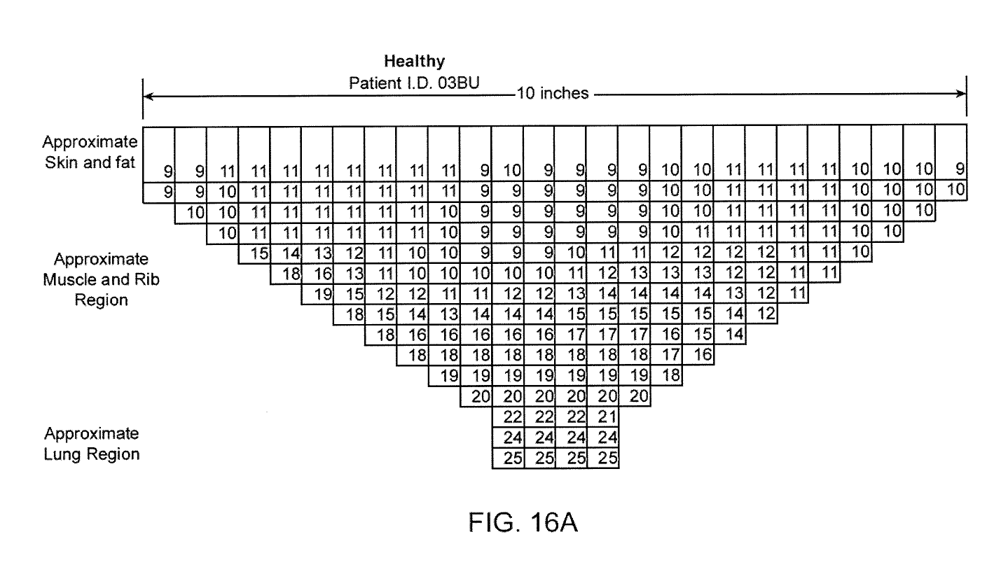

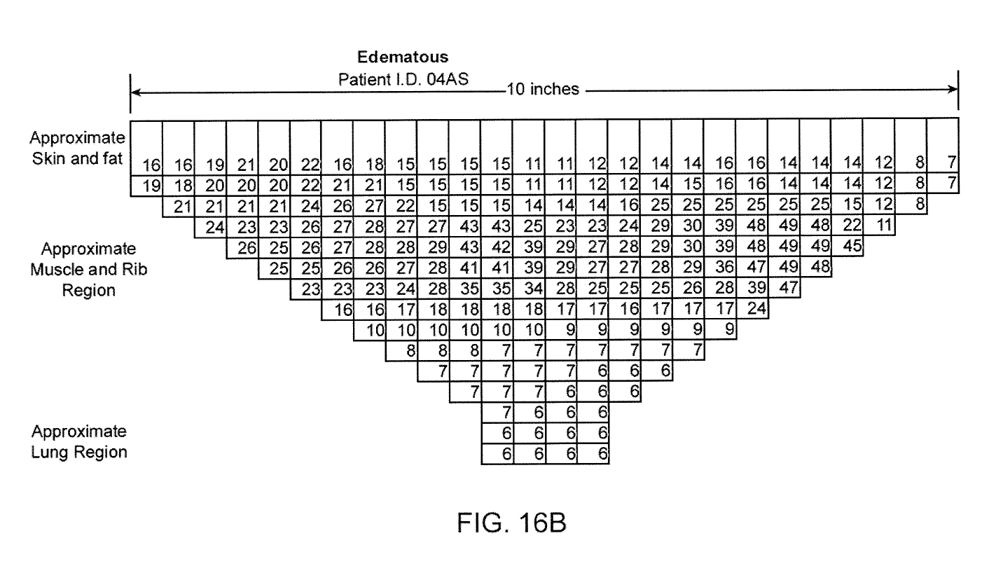

In the examples described herein, the range of low frequency applied is approximately 80 kHz or less (e.g., 50 kHz or less, 30 kHz or less, than 20 kHz or less, 10 kHz or less, etc.). For example, 20 kHz is used in the examples shown in FIGS. 16A and 16B, described below. The high frequency range is typically about 100 kHz or greater (e.g., 200 kHz or more, etc.).

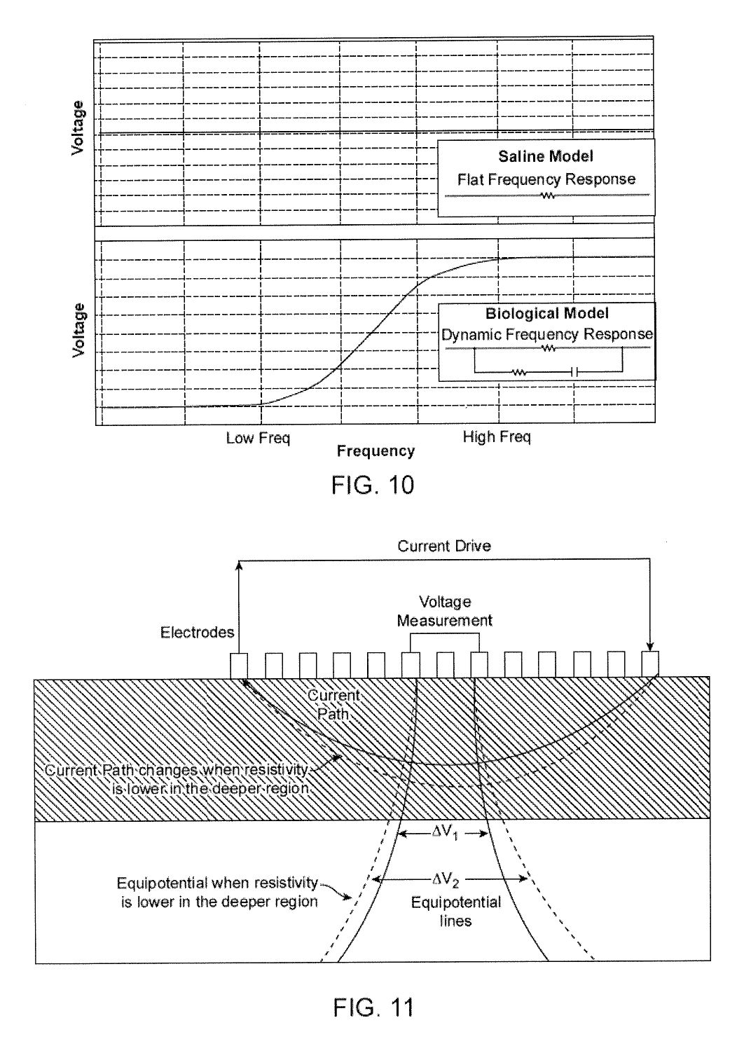

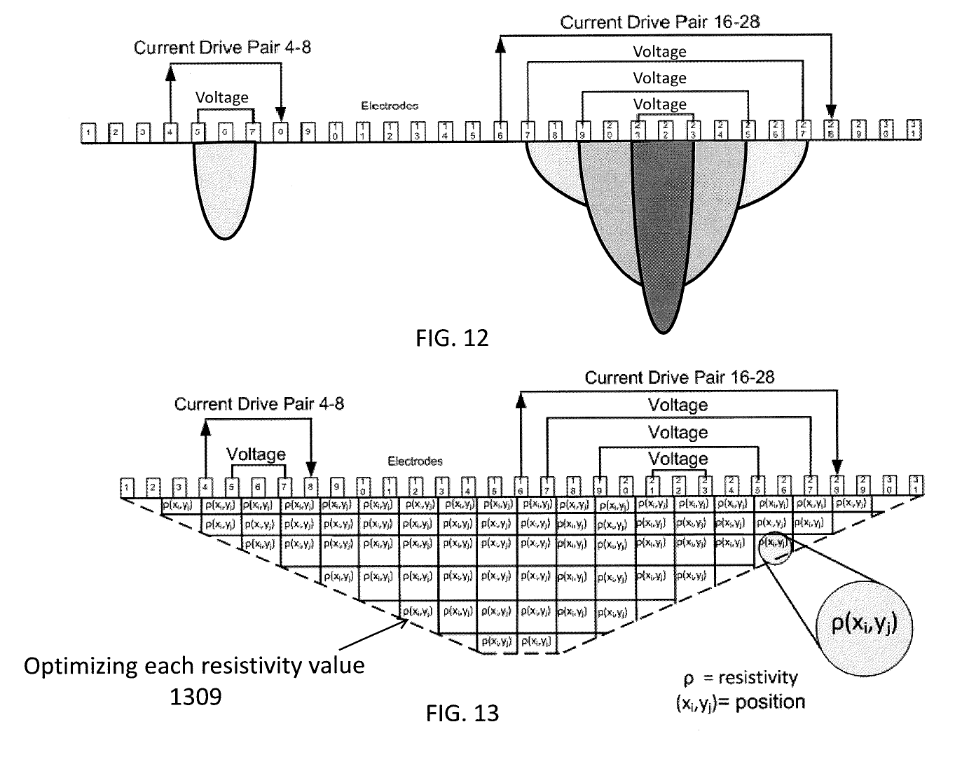

FIG. 11 illustrates an example of an electrical resistivity array positioned over tissue to examine apparent resistivities indicating different tissue regions. In FIG. 11, the surface of the body includes an array of electrodes arranged so that the current is driven between outer electrodes and voltage is measured across pairs of inner electrodes. The current path between the drive electrodes changes from the solid line current path to the dashed line current path when resistivity is lower in deeper regions of the body. Similarly, the orthogonal equipotential lines between pairs of sensing electrodes separate further (larger delta-V) when the resistivity is lower in the deeper regions. Thus, changes in resistivity in the region beneath the tissue at various depths may be detected as changes in the voltage measured at various sensing pairs. On this basis, given a known array configuration, known applied current and measured voltages, the system may be optimized to solve for the resistivities in various depths beneath the electrodes. The depth that can be detected may depend on the arrangement of the electrodes on the surface, as illustrated in FIG. 12.

For the systems and methods to determine lung wetness as described herein, it is particularly of interest that the applied current and detected voltage allows sensing at sufficient depth to reach the lung. Thus, the application of the electrodes in the appropriate position is important, as is the manner in which the drive currents and sense voltages are applied and received. For example, in FIG. 12, an exemplary device having 31 electrodes (drive electrodes alternating with sensing electrodes) is used to illustrate the depth sensitivity that can be achieved depending on the arrangement of the drive and sensing pairs. The combination of both shallow and deep measurements may therefore allow a reconstruction of the distribution of the spatial arrangement of resistivities. In the examples provided herein, voltage is measured between drive electrodes, and the combinations of drive and sensing electrodes may be varied to achieve some spatial arrangement shown in the figures (e.g., FIGS. 16A and 16B). For ease in the examples, the voltages are sensed from the odd numbered electrodes (e.g., FIG. 12) while current is driven from the even electrodes. For example, in FIG. 16, 252 electrical resistivity arrays where used. Although we describe the use of driving electrodes and driving currents herein, it should be apparent that the applied current may also be measured. For example, the current may be measured across a region in-line with the electrode (e.g., through the electrode). The complex current may be measured.

FIG. 13 shows another example of the construction of a distribution of resistivities based on a multitude of electrical resistivity arrays. As mentioned above, when internal resistivities are known, it is possible to solve for the surface voltage; the inverse relationship may also be used (as here), to solve for internal resistivities given surface voltages. Thus, a heat map representing the distribution of the resistivities may be generated by optimizing to fit the sensed voltages given the drive current and the known geometry of the array. In general, solving for the internal resistivities from surface voltages may be performed by solving a combination of forward and inverse problems. The relationship between sensed voltage, applied current and internal resistivities may be expressed as a Poisson equation. Although the geometry beneath the tissue surface is initially unknown, the measured voltage and known currents at different frequencies may be used to optimize within acceptable error tolerances to determine the resistivity of the sub-surfaces. This inverse problem may be iteratively solved and the results fed back into the forward problem to minimize the misfit error between the measured and computed resistivity values on the surface.

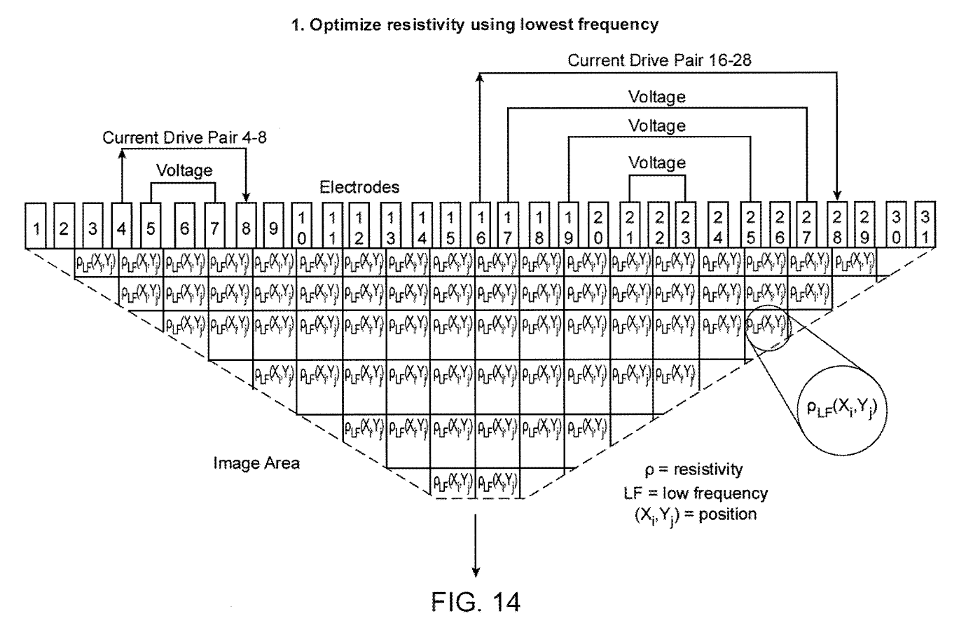

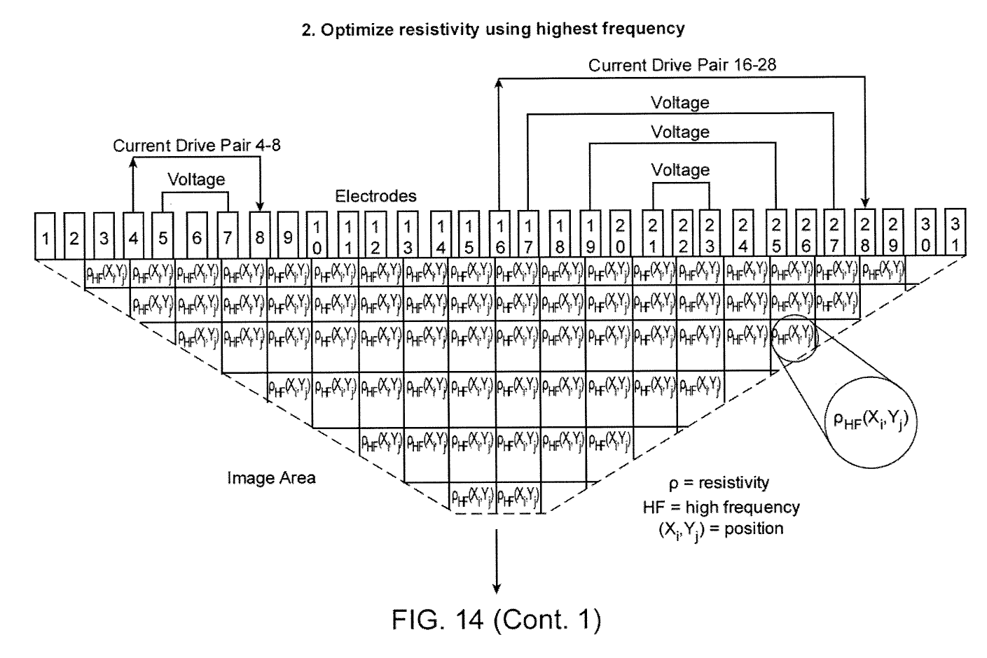

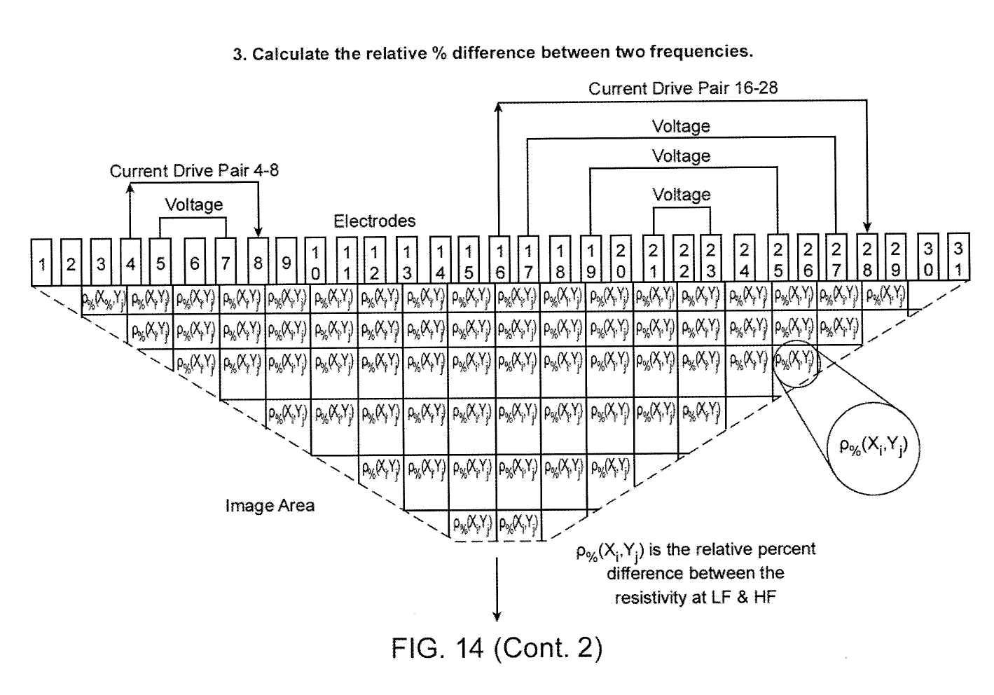

FIG. 14 illustrates one method of determining a distribution of relative percent differences in order to determine lung wetness. For example, in FIG. 14, the distribution of resistivities at a first (e.g., lowest) frequency is performed in step 1. In step 2, the distribution of resistivities at a second (e.g., highest) frequency is performed for the same volume. Finally, at step 3, the relative percent differences are calculated between the two frequencies (using the information of step 2 and step 3).

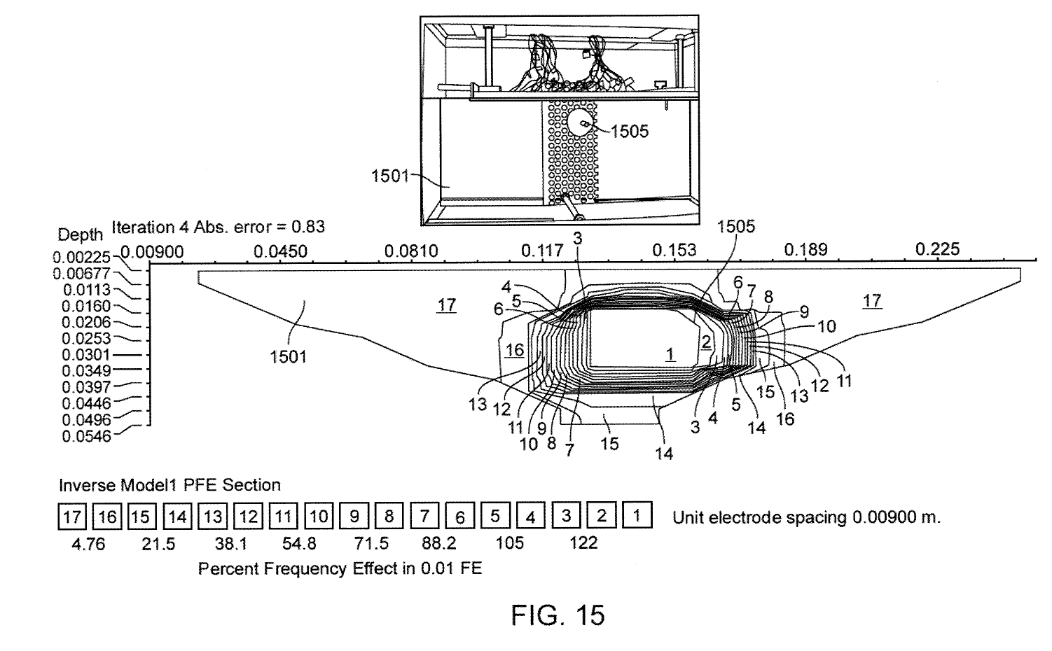

An example of this method performed as a proof of principle is shown in FIG. 15. In this example, a saline tank holding a submerged biological tissue (yam) is used with an array of electrodes near the top of the tank to confirm that the change in resistivity between the water and the biological tissue can be visualized. The distribution shown in the bottom of FIG. 15 illustrates the relative percent differences between a high (200 kHz) and low (20 kHz) kHz frequency for the yam in saline. The image is pseudo-colored, and shows that the boundary of the yam and water is readily distinguished, and as predicted, the biological tissue (yam) has a much larger relative percent difference than the water (which, as discussed above, is virtually flat).

FIGS. 16A and 16B illustrate one example of the application of the methods and systems described herein for detection of lung wetness. FIG. 16A shows a distribution of the relative percent differences of the resistivities between a high frequency and low frequency for a healthy (“dry” lung) subject. For comparison, FIG. 16B shows the distribution of relative percent differences of the resistivities between a high frequency and a low frequency for a wet (edematous) subject. The images may be analyzed to illustrate how to identify lung wetness.

A variety of tests may be used to interpret the distribution of resistivities, and particularly the distribution of relative percent differences of resistivities, to indicate or track lung wetness in a subject. For example, in some variations a series of tests or analyses of the distribution of relative percent differences may be performed in order to determine if a lung is wet or dry.

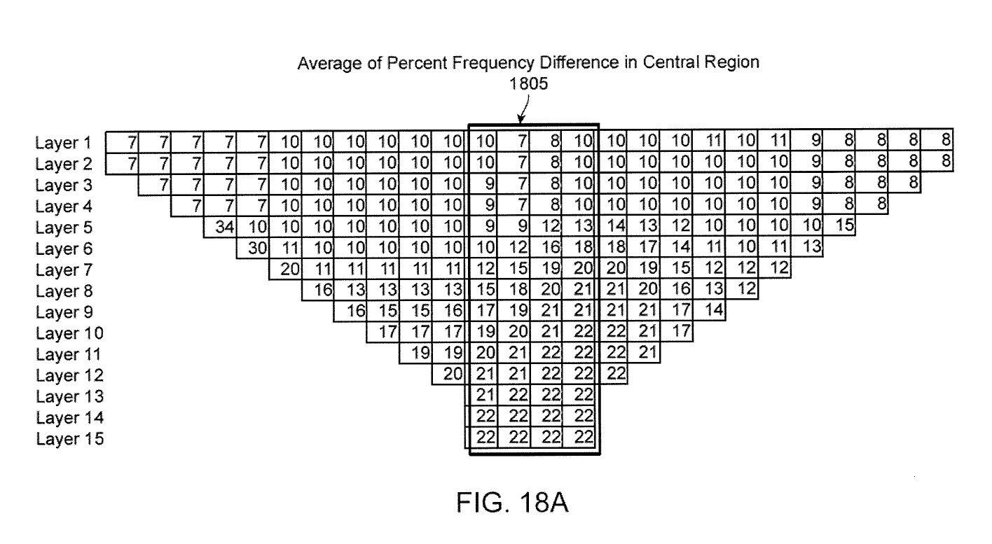

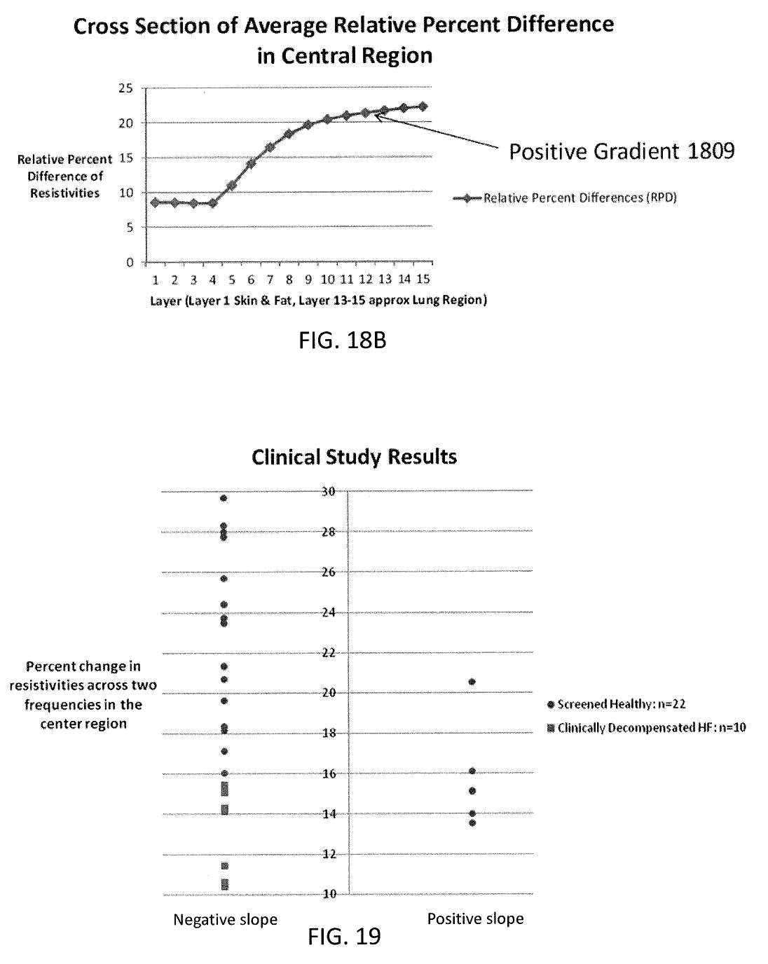

For example, in one variation, the change in relative percent differences in the distribution with increasing depth into the tissue (e.g., from the outer skin surface, through the muscle and into the lung) may be examined to determine lung wetness. In this test of analysis, if the change in relative percent difference is increasing (e.g., if the slope of the lung wetness is positive) as the depth increases towards the lung, it is likely that the subject is dry (e.g., has a “dry” lung). This is illustrated in FIGS. 18A and 18B. In FIG. 18A, a central region of the distribution (shown by the central rectangle showing a 4×15 box) is examined. For each layer (roughly corresponding to depths of penetration into the tissue) an average value (the average of the four shown for that layer) of the relative percent differences is taken, and this average is plotted as shown in FIG. 18B. The resulting graph has a positive slope.

Another test that may be applied is shown in FIG. 19, in which an overall average value is extracted from the distribution of relative percent differences. In this example, an average of the central region (e.g., the boxed region shown in FIG. 18A) is taken and compared to a threshold value. If the average is above the threshold value, it is likely that the lung is dry; if the value is below the threshold value, the lung may be wet (although additional tests may be applied). In FIG. 19, average percent changes in resistivities between a high and low frequency taken from the central region of the distribution were plotted for both healthy (dry) subjects and edematous (wet) subjects. In the example shown in FIG. 19, the wet subjects all fell below a threshold of under 16, whereas all of the healthy (dry) subjects that had non-positive (e.g., negative, flat) slopes (for the change in relative percent difference from the central region) fell above this threshold. It should be appreciated that the threshold shown here is merely exemplary. The appropriate threshold value may vary depending on various factors, such as the manner in which the average value is determined, the configuration of the array, the high and low frequencies used to generate the RPD, and the like, the general principle of a threshold value remains. The actual numeric value of the threshold may therefore be empirically determined using similar parameters across a variety of dry and wet subjects. In some variations, the threshold value is not a strict cut-off, but may include a range of values; if the average (or in some variations the sum) value of the RPD is within this range; the lung may be wet or may be indeterminate.

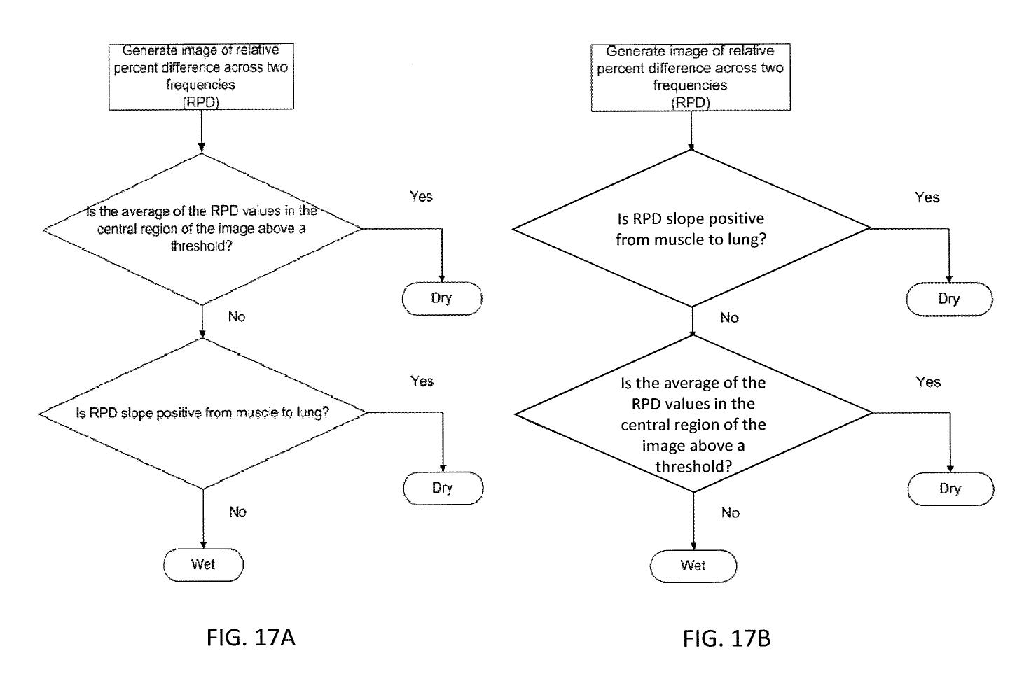

As mentioned above, a combination of different tests may be used to determine lung wetness. For example, in some variations an individual test alone is not conclusive, but different tests may be performed sequentially or in parallel to provide a higher degree of confidence of lung wetness. For example, FIGS. 17A and 17B illustrate variation of methods for testing to determine lung wetness using the individual tests described above. For example, in FIG. 17A, a distribution of relative percent differences between high and low frequencies may be serially examined to determine lung wetness. In this example, the first test performed is the comparison of the average RPD from the center of the distribution (including the “deepest” region). If the average RPD value is above the threshold (e.g., >about 16), then the subject's lung is considered “dry;” if the average RPD value is below the threshold (e.g., <about 16), then the second test (looking at the slope as illustrated in FIGS. 18A and 18B) is performed. The order of these tests may be switched in some variations, as shown in FIG. 17B.

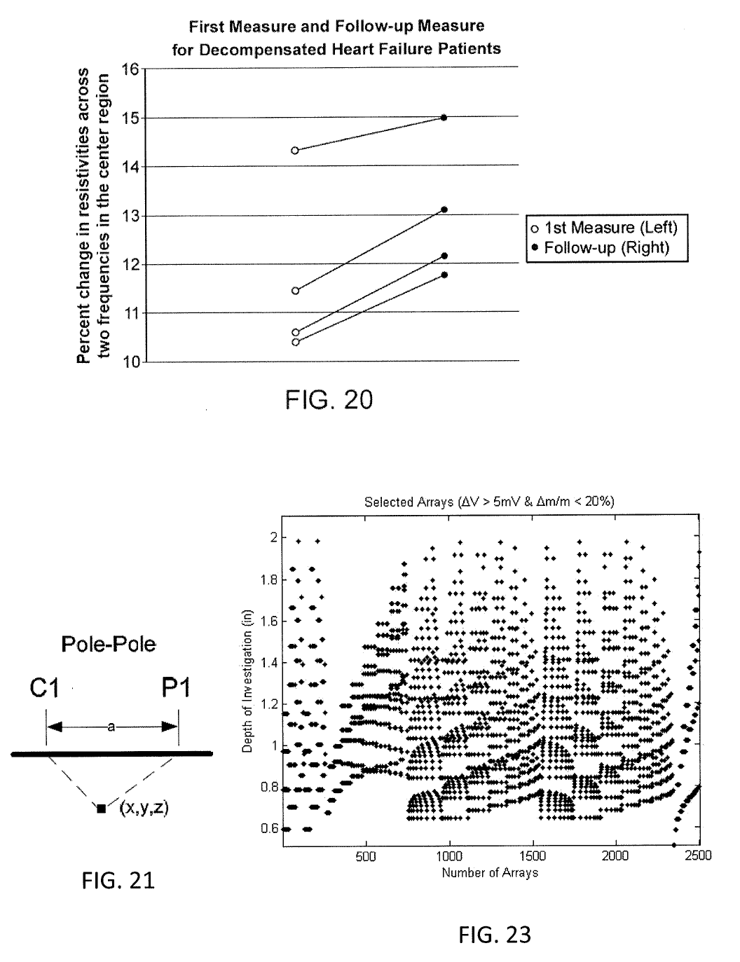

Any of the methods and systems described herein may also be used to examine the clinical progression of lung wetness, including monitoring of treatments for lung wetness (e.g., diuretic treatments, etc.). An example of this is illustrated in FIG. 20. In this example, four subjects having lung wetness were monitored over treatment. Lung wetness by assessed by the techniques described herein (including the generation of a distribution of RPD), and was configured by classical diagnostic methods, including listening (auscultation) for rales or characteristic “crackling” sound linked to excessive fluid in the airways. All four subjects initially had average RPD's from the central region of the distribution that were below the threshold, indicating lung wetness. During the course of treatment, as shown in the “follow up” column on the right side of FIG. 20, this average value increased in all subjects, however, all of these subjects remained below the threshold. Interestingly, these subjects also showed an improvement in their lung wetness and in some cases no longer displayed some of the more classical characteristics of lung wetness such as rales during respiration. Although these subjects showed some improvements, other measures of overall lung wetness (such as swollen ankles, etc.) remained. This may indicate the relative sensitivity of the present systems and methods, particularly in tracking the treatment of lung wetness, compared to more traditional methods.

In some variations, the electronics controlling the acquisition of data (e.g., driving the current and recording the voltages are contained as part of a system that is connected directly to the patch (sensors) having the array of electrodes. In some variations the drive/read electronics are miniaturized and configured to be “worn” on the patch/sensor with the electrodes when applied to the patient.

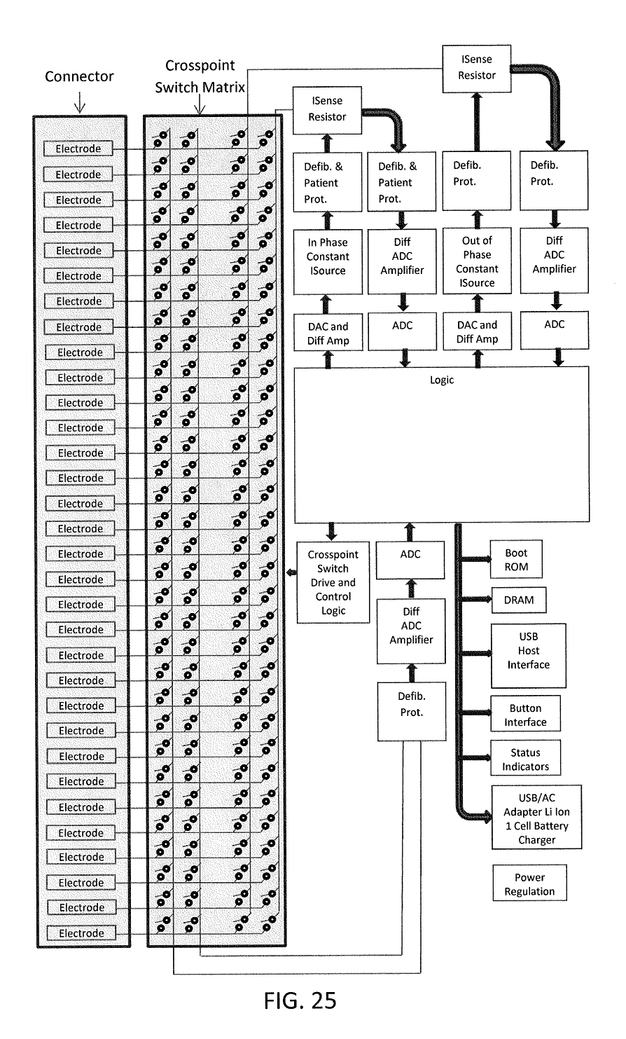

FIG. 25 illustrates one variation of a schematic for a variation of drive/read electronics that can be miniaturize and configured to reside on the patch containing the electrodes. This miniaturized hardware may have a connector and be connected to a battery (e.g., a small Li Ion battery) and also directly connected to the patch including the electrodes. There are numerous advantages to this embodiment, including removing the necessity for a harness, and potentially improving the accuracy because the impedance measurements will not pick up any impedance due to a (somewhat inductive) harness.

In FIG. 25, the exemplary system includes 31 electrodes similar to the example discussed above. In some variations, however, rather than requiring separate circuitry such as a separate motherboard (e.g., for multiple modules each for regulating driving/reading off a separate electrode) and two or more batteries, the variation shown in FIG. 25 has been integrated so that there are 31 electrode and only two drive and two measurement modules that are multiplexed (MUX, shown as a crosspoint switch matrix in FIG. 25) to provide a constant current source having a source electrode and a sink electrode.

In the example shown in FIG. 25, each of the 31 electrodes is competent to act as a voltage sense (either side) or as a constant current source/sink. In particular, the constant current source may apply current from a source as well as a sink; the current at the sink is 180 degrees out-of-phase with reference to the current source. Alternatively, in some variations instead of a the electrode being configured as a current sink applying current 180 degrees out of phase, the electrode may be configured as a floating ground. Returning to FIG. 25, the two columns on the far right of the crosspoint switch matrix may be selected to configure a particular electrode as either a current source or a current sink. In this arrangement, the current is driven differentially so that it acts as a differential voltage/current source and current sink. The rows of the crosspoint switch matrix furthest to the left may be selected to configure an electrode as part of a voltage sensing pair. FIG. 25 shows the MUX as a crosspoint switch matrix, however any appropriate multiplexer may be used.

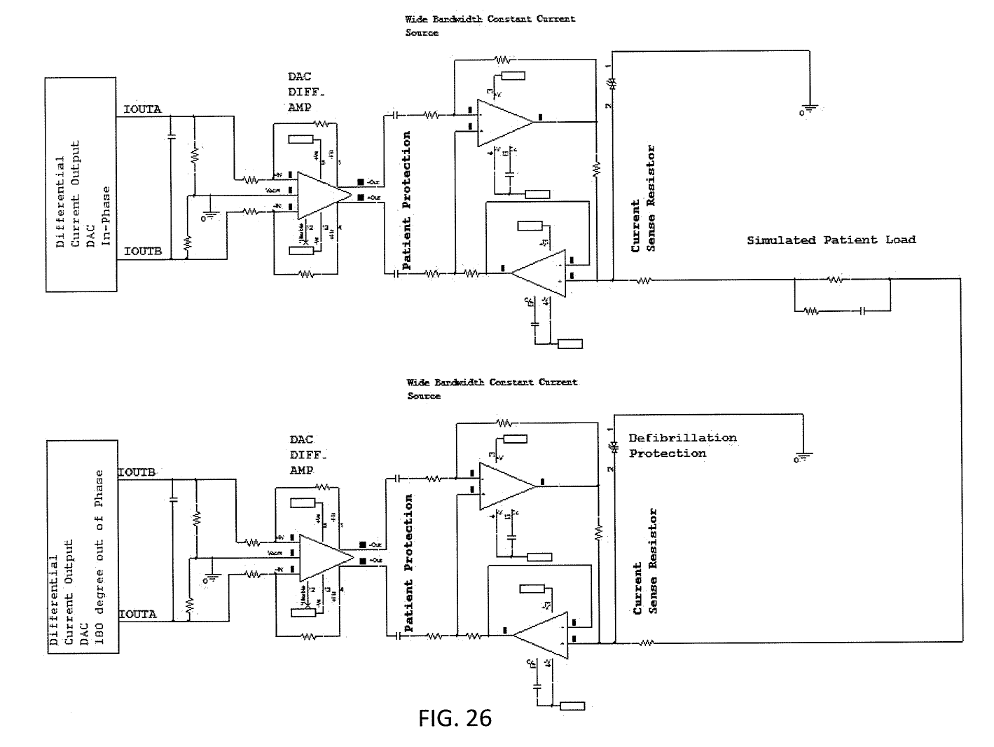

The constant voltage source illustrated here and in greater detail in FIG. 26, discussed below, has numerous advantages over systems that do not apply a constant current (e.g., constant voltage sources) for applying current from the drive electrodes. For example, individual impedances (e.g., the drive pair impedance, aka the impedance seen by drive pair) typically varies from patient to patient, possibly based on the hydration state of the patient's skin, the thickness of muscle, skin, etc. layers, and the like. If a power source other than a constant current source is used (e.g., constant voltage), the system may put in much lower current (and therefore power) than would otherwise be used. In contrast, putting in a constant current allows a larger current signal to be delivered and therefore received, and results in a larger output, and therefore a larger signal to noise ratio (SNR). As will be discussed in part III, a constant current source may also help with electrode reciprocity. Reciprocity refers to the ability of a four-point electrode resistivity array (e.g., sensor element) to show similar output when the drive electrodes are switched with the sense electrodes. Switching the drive and listening (sense) electrodes should result in a similar (if not identical) signal; if it doesn't, then there is no reciprocity among the electrodes forming the four points, and there may be a problem with the electrodes, such as a problem with the contact between the electrodes and the patient's skin. Thus, reciprocity can then be used to check the system, or the electrode reliability and quality.

It is not necessary for a constant current source to be bipolar, providing both source and sink currents. In some implementations, the constant current source uses a floating ground. The use of a bipolar (source/sink) driver may help reduce noise effects in the system. Also, having two modules to apply current gives you two additional locations to check the output of the constant current source.

In FIG. 25, the reference to the defib (or defibrillator) refers to circuitry that may be included in the apparatus for handling current due to defibrillation of a patient wearing the apparatus. The defibrillation and patient protection circuitry may prevent damage to the patient or the equipment connected to the patient in the event the patient is defibrillated while wearing the apparatus.

The multiplexed system shown in FIG. 25 may be miniaturized, and may provide relatively quick response time in use. For example, each voltage measurement made at an electrode combination may take 1 ms or less (e.g., ½ ms), depending in part on the filter settle times. Thus, it may take approximately 1 sec to go detect 1000 measurements at particular frequency. It is therefore possible to make multiple scans (and to average the scans) and make scans at a variety of different frequencies within a reasonable time period (such as a few minutes).

The system may also be configured to filter low frequency (e.g., 50 and 60 Hz) noise using one or more filters, including custom filters. For example the system may include a filter or filters to eliminate the low frequency noise using a small sample window.

The exemplary system in FIG. 25 also indicates multiple possible outputs, including visual/audible outputs for communicating with a user and/or patient. For example, the system may include a display/screen (e.g., a touchscreen) for presenting visual information showing the progress of the procedure, and/or for presenting information about the electrodes, such as electrode contact quality, positioning of the electrodes, timing, etc. or instructions for applying the sensors, and/or taking the measurements.

In some variations of the apparatus, including miniature, wearable apparatuses, the apparatus include additional processors for analyzing/processing the data to determine tissue wetness (e.g., lung wetness) and/or an indicator of hydration status/wetness.

In the variation shown in FIG. 25, the apparatus includes a memory for recoding/holding the information collected, and one or more output means (e.g., USB host interface, wireless transmitter, data bus, etc.) for transferring the data to other devices. In some variations the apparatus may also or alternatively be connected directly (wired) or wirelessly. The data may be transported to a computer or other microprocessor including analysis logic sufficient to interpret the data and determine the RSCSRAF and/or output an indicator of tissue wetness. In some variations the microprocessor is a mobile communications device including a cell phone, tablet, pad (e.g., iPad™), or the like.

As mentioned above, in general the power source driving the drive electrodes may be a constant current source. For example, in some variations the source is a bipolar, differential, voltage controlled constant current source. FIG. 26 shows an exemplary schematic of such a constant current source. In this example, the circuit is symmetrically configured, as shown, although other configurations are possible. In this example, the circuitry on one side provides output to the source, while the circuitry on the other side provides output to the sink, and the current generated is 180 degrees out-of-phase with the current on the source side. In this example, multiple digital to analog converters with low noise, wideband are used (such as Analog Devices ADA4940) to drive the voltage differential controlled current source. The amount of current may be set using a voltage source, and the current remains constant. The exemplary source in FIG. 26 can also provide multi-tone (e.g., multiple frequencies) and maintain a constant current. Thus, two or more tones (e.g., 200 KHz and 20 KHz) may be simultaneously sent at a constant current. The amplitude of each tone may be set using the device. A bipolar, differential, voltage controlled constant current can be used to perform reciprocity measurements to test measurement integrity, as mentioned above. Further, electrode skin impedance does not affect the apparent resistivity, and the source is able to maintain same current across frequencies.

### Layered Parameter Model

Various methods for determining if a tissue, and particularly if lung tissue, is “wet” or “dry” are described herein. One method of determining relative wetness uses a layered parameter model. In this variation, a layered model is used in which multiple layers with potentially different thicknesses and conductivities. Initially, data is collected from patients that are pre-categorized as either wet or dry. Data from the arrays of electrodes (a patch) for each patient may be fit to sets of parameters, and the parameters may be compared to see if there is a change in the parameters between layers of dry vs. wet patients. Note that alternative models, such as a Cole-Cole type of model, don't include “depth” as a parameter. Thus in a layered model, the model includes a depth of investigation as a parameter. An analytic model for a rectangular box with different layers (e.g., 3 layers or more) can be solved. This technique is similar to the inverse problem discussed above. The model can be tuned until the voltages on the surface match the data. The layered model, because of the simple geometry and constant depth, allow optimization over six parameters (rather than the number of voxels).

For example, measurements may be taken across two frequencies and their associated RPD values may be calculated. Using a two or three layered half-space model, with parameters h1, ρ1, ρ2, or h1, h2, ρ1, ρ2, ρ3, respectively, the data may be fit to distributions associated with each parameter based on the RPD values. The fits will not be perfect, so possible parameter distributions may be determined. Movement of the distributions across both models may be tracked, and a two or three layer model based on Aiken or Bayesian information criterion may be chosen. Using the difference between the wet and dry subject's distributions, a hydration index may be tracked by a layered parameter model. By replacing the analytic model with a numerical model, the layers can have variable width.

### Detecting Thorax Size

In general, boundaries descried herein may be modeled by approximations of the patient's anatomy or by simplified (e.g., box) models. As long as we use finite models, we have to make assumptions about the geometry (of torso, etc.). However, in some variations it may be better to treat the size of the model as another parameter, and to imagine that subjects are homogenous and find the size of box that best fits their measurements. The apparent size of the thorax can be used as an additional parametric model to differentiate between subjects. The rectangular model assumes a homogenous material of size W*L*D with a set of tetra-polar electrodes running in-line. The parametric task is to find the parameters W, L, D, ρ and array center, given the resistivity measurements. The homogenous assumption used in the model may not apply at lower frequencies when the internal organs in the thorax become electrically heterogeneous affecting the parameters distributions. It is the disturbance in the parameter distributions that differentiate subjects.

### Pseudo-Spectral Forward Problem

The discretization of the PDE analysis discussed is usually performed in terms of finite element or finite volume methods, as this provides a natural coupling between the material voxel properties and the fields passing through them. However, both these methods have algebraic accuracy. If the domain used in the forward problem continues to be a simple rectangle, a pseudo-spectral method would instead provide exponentially accuracy with the added advantage of having FFT complexity.

### Part II: Sensor/Electrodes

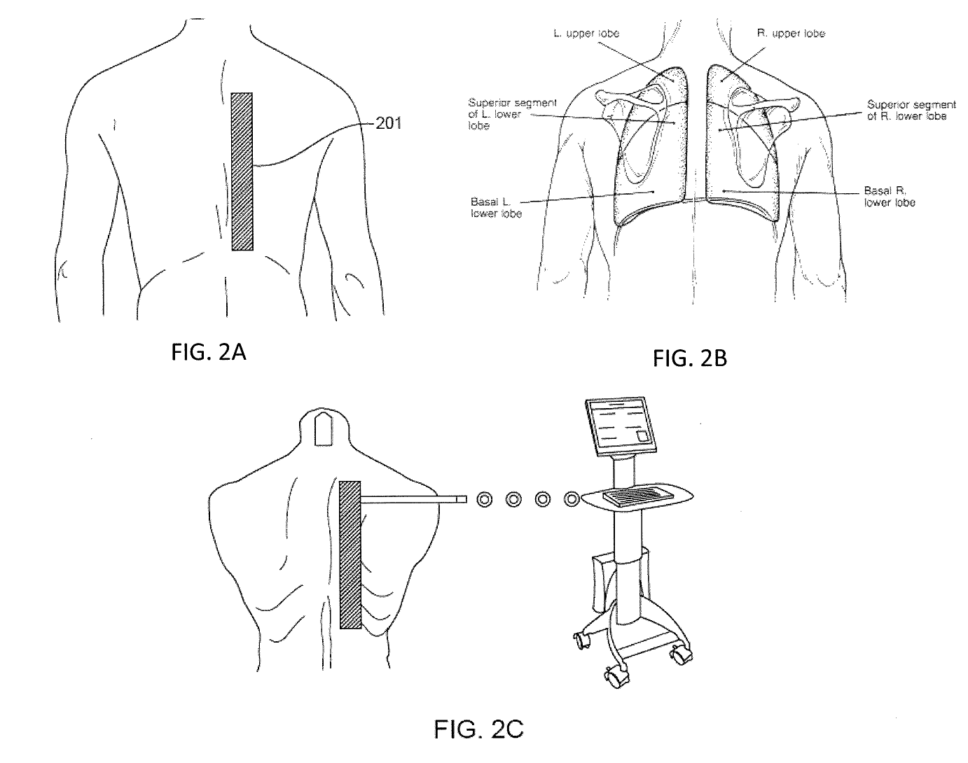

In general, a sensor defined by an array of electrodes (e.g., a strip array of electrodes) may be placed on the subject's back in a particularly arrangement allowing for detection of lung wetness. For example, FIG. 2A shows one example of a sensor (configured as a strip of electrodes) positioned on a region of the subject's back (to the right or left side of the subject's midline on their back, lateral to the spine). The sensor 201 (an array of electrodes) may be applied locally in just one region of the subject's back. In FIG. 2A, the electrodes positioned in particular near the midline of the subject's back may be positioned immediately over the lung (either the right or left lung). This may be achieved by placing the top electrode in the sensor in line with the top of the subject's scapula, while extending the rest of the electrodes in the active region of the sensor down the back (cranially to caudally) as shown.

In some of the examples provided herein, the electrodes are adapted for placement on a subject's thorax (e.g. posterior and anterior region of the body) for determining the distribution of resistivities immediately below the array of electrodes (e.g., the skin, muscle and lung tissue). The systems described herein may provide information to aid in determining the fluid content of a lung, which may be relatively deep within the body when compared with skin and muscle. In FIG. 2A, the sensors (array of electrodes) are positioned so that it may be possible to measure from the posterior region of the right lung. In this example the array of electrodes (an example of which is provided below) are positioned about 1 inch lateral of spine, as shown in FIG. 2A. This location may allow depths of investigation to reach the posterior region of the right lung.

In many of the variations of the sensor described herein, the electrodes are arranged on a strip, patch or other fixed arrangement that can be positioned on the skin of the subject. For example, the strip of the electrodes may be an adhesive strip that can be positioned to one side of the subject (e.g., one side of the subject's back). This configuration may allow for sensing a depth beneath the array of electrodes, and therefore determining the arrangement of the resistivities beneath the electrodes. In this arrangement, as opposed to a strap or band of electrodes, the arrangement of electrodes may be fixed relative to each other, so that the geometries between electrodes is fixed and known. Such local electrode positioning may have numerous advantages.

The arrangements, including the spacing, of the drive and sensing electrodes within an electrical resistivity array may be configured to allow sensing both at depth (e.g., deep within the tissue) and at more superficial regions (immediately beneath the electrodes). Part III (below) describes designing a sensor (a combined electrical resistivity array) for determining tissue wetness, as well as devices and methods for selecting which electrical resistivity arrays to be used. The measurements described herein may be made on a subject instructed to sit or lie in a particular posture. For example, when taking lung wetness measurements, the subject may be instructed to lie on his or her belly, lying prone (on his or her back), or sit reclined at an angle (e.g., 45°) when taking the measurement. In some variations the subject may be asked to assume the same position when taking measurements at different times. Typically, in heart failure the lungs get wet, and the weight of the fluid in the lungs may alter its distribution. For example, in the lungs, the weight of the fluid may partially compress or collapse the lower region of lung. Posturally, it may be desirable to have the subject lie supine or nearly supine; this may help make the lung wetness easier to measure, particularly when placing electrodes on the subject's back

In general, a sensor may contain a plurality of electrodes, typically of both drive electrode and sensing electrode types. The drive and sensing electrodes may be identical, or may have different geometries. In some variations, the same electrodes may be used to both drive and sense (at different times or simultaneously). In general, the electrodes may be electrically connected to the rest of the system via a wired or wireless connection (not shown in FIGS. 2A and 2C).

FIG. 2C illustrates one example of a system for measuring lung wetness. In this example, the system includes a disposable sensor that is connected to a reusable monitoring/processing station. The monitoring/processing station includes a controller for regulating the application of drive current and coordinating sensing from the sensing electrodes and processing of the received signals. The system may also include one or more processors for determining a spatial representation of resistivities beneath the sensor of electrodes at different applied current frequencies. The same, or a different, processor may also determine a spatial mapping of relative spatial change in subsurface resistivity across two or more frequencies (e.g., a high frequency and low frequency) and may then determine lung wetness based on the RSCSRAF.

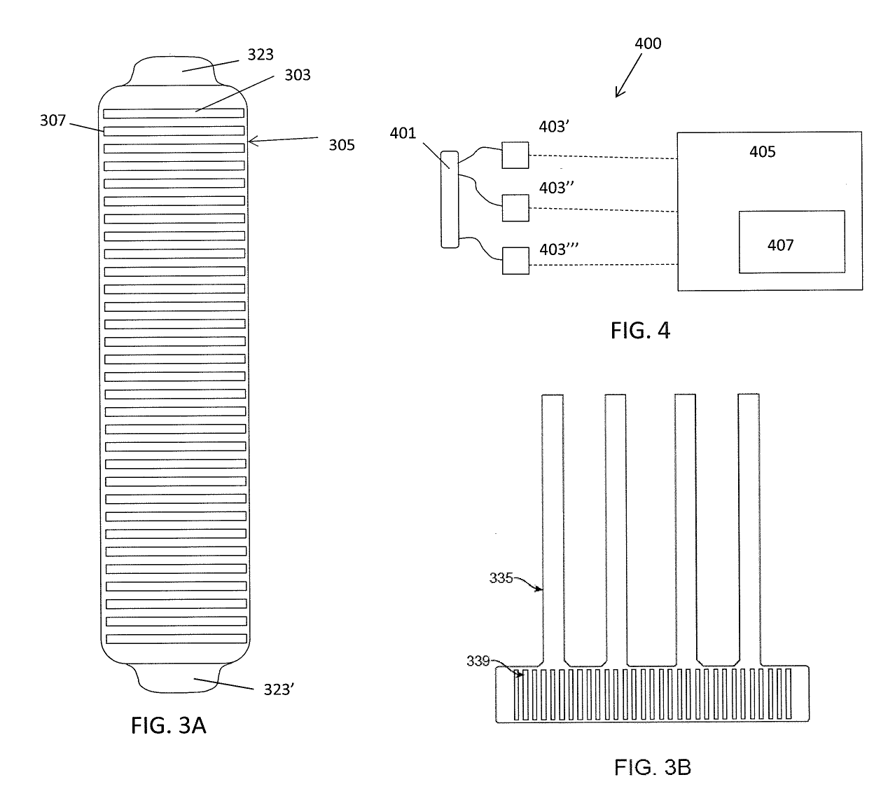

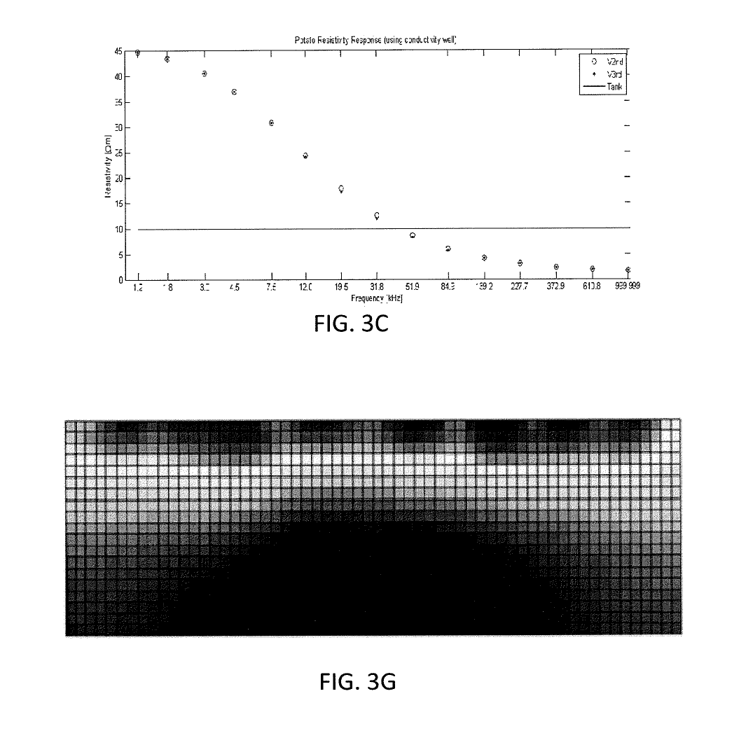

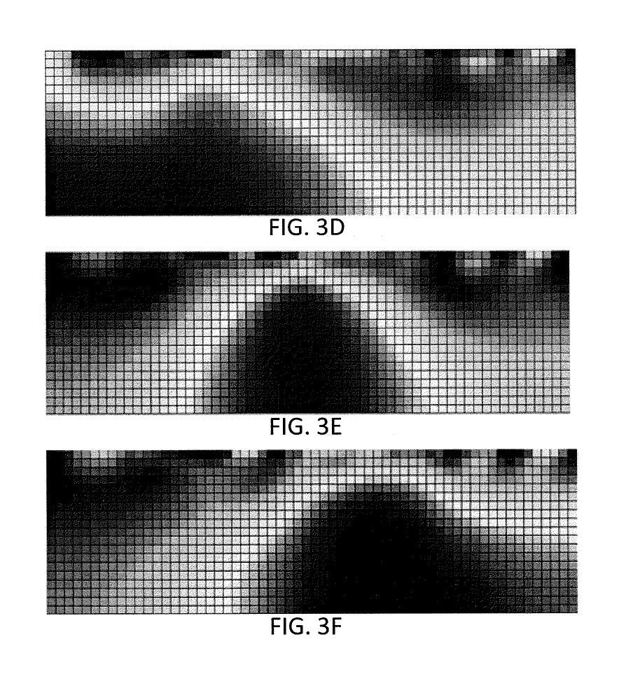

FIG. 3A illustrates one variation of a strip array (sensor) that may be used. For example, in FIG. 3A the electrode array 305 includes drive electrodes 303 alternating with sensing electrodes 307. In this example a total of 31 electrodes are included in the array, with the electrodes arranged down the length of the array. Each electrode in this example is rectangular in shape. The array in this example is configured as a sensor that includes a hydrogel that may be applied directly to a subject (e.g., the subject's back). In some variations the lateral edges of the electrodes extend to the edge (or almost to the edge) of the sensor. Thus, in some variations, as shown in FIG. 3A, the sensor includes a proximal 323 and distal 323′ grip or holding region for grasping to apply the sensor. In some variations the patch or sensor array may include one or more indicators and/or graphics to aid in placement and/or orientation. For example, the patch or sensor may include a graphic that indicates the middle of the patch or sensor.

In one variation the patch is formed to include a plurality of electrodes attached to a polyester backing (including a titanium powder for bacteria resistance). The patch may be formed of a medical grade dielectric material that is UV cured onto the bottom. The electrodes may be connected by insulated vias (e.g., connectors, not shown). In some variations, the arrangement of the electrodes of the patch is predetermined, and matched to the processor of the system. For example, the patch may have a “standard” arrangement that is used by the system, or there may be a variety of patch electrode configurations from which the system may select to match the arrangement of the actual patch to be used. Thus, the system or device may include information describing and corresponding to the arrangement/configuration of the electrodes (including electrode numbers, sizes, etc.) in a particular type of patch, or the patch may itself provide this arrangement/configuration to the rest of the system, so that it can be passed onto the processor and used by the system in determining the distribution of RSCSRAF as described below. For example, a sensor may include a chip or other identifier that confers this information to the rest of the system.

Another example of a patch design including a plurality of drive and sensing electrodes is shown in FIG. 3B. In this example the patch shows four regions from which the leads 335 extend from the electrodes 339. The leads are typically insulated and configured to connect with the rest of the system. Although this example shows four bundles of leads extending laterally from the patch, in some variations fewer or more bundles of leads (e.g., 1, 2, 3, etc.) may be used. The lead bundles may be fabricated as part of the fabrication processes (e.g., photolithographically, screen printing, etc.).

As mentioned above, to determine lung wetness, the sensor (e.g., patch) may be applied to the user's back in a position that is laterally offset from the spine (e.g., midline of the back) by approximately 1 inch, so that the lateral edge of the electrodes is 1 inch from the midline. The top electrode in the patch may be lined up with top of scapula, so that the remaining electrodes run parallel down the back, transverse to the midline, as shown in FIGS. 2A and 2C. In these examples, the active region of the patch extends approximately 11 (e.g., 10.8) inches, so that the electrodes span this distance down the cranial-caudal axis of the back. The electrode spacing is approximately 0.36 inches center to center in the example shown in FIG. 3B, and the electrodes are rectangular, having a width of approximately 0.15 inches and an elongate length of approximately 2 inches. Although other geometries may be used, in general, these geometries are optimized for the patch because they allow a relatively large surface area for the electrode, while making consistent and complete contact with the subject's back, which is often curved or irregularly shaped. Thus, it may be beneficial to have the active region of the patch be between about 8 and 12 (e.g., 10) inches long and between about 1.5 and about 2.5 (e.g., 2) inches wide. The lateral amount of support backing on either side of the active region may be minimized as well. Minimizing the amount of lateral material may prevent the patch from buckling, wrinkling, or puckering as it is applied/worn. The relatively narrow width of the patch may help it to conform to the contour of skin. Overall the patch is flexible, and in the examples shown in FIGS. 3A and 3B, has a thickness of less than about 3 mils, which may also help it conform to the body contour.

In some variations, the sizes and shapes of the electrodes may be selected to optimize the sensing ability of the system. For example, the area of each electrode in the sensor may be selected to allow sufficient current to be delivered so to increase the signal to noise ratio on measurements taken. The electrodes may be long and narrow to allow close spacing between the electrodes to provide sufficient resolution, while the length may be sufficiently large so that the current density can be sufficiently low. The width of the electrodes may be limited to allow detection of the tissue beneath the electrodes between the spine and the scapula, so that the electrodes may be placed over this narrow region of the body that allows a “window” to detect the lungs. The spacing may also be important to allow sufficient depth of penetration/sensing into the subject's body. The arrangement shown in FIG. 3A, for example, is configured to allow a depth of investigation of approximately 2 to 2.5 inches. The electrically conductive members (electrodes) do not contact each other, and are spaced adjacent, but sufficiently far enough apart to prevent electrical coupling while allowing the electrodes to measure the voltage seen by the current applied by the current-injecting or driving electrodes.

In practice, the systems may be configured to view the lung, and thus may be placed as illustrated in FIG. 2A, by securing them to the subject's back approximately 1 inch lateral of the spine, between the spine and scapula. The linear electrode array is placed up/down relative the subject's body (e.g., cranial to caudal, from head to feet) in parallel to the spine. The strip of electrodes may be placed as high as possible (e.g., to the level of the armpit). The electrode strip may be used with, or may include a conductive gel (e.g. hydrogel) that helps make the electrical connection with the skin. The strip could be placed either on the left or right side of the spine.

As mentioned above, the structure of the array is predetermined. Thus, the patch controls the spacing between electrodes. In this example, the patch of electrodes is approximately 2 inches wide (e.g., 150 mm wide). The example of FIG. 3A shows alternating drive and sense electrodes. In this example, there are 31 electrodes; 16 of them are voltage sensing (listen) electrodes and 15 are current drive electrodes.

To test the concept of RSCSRAF to detect a biological structure in the subsurface of the patch, a potato was placed in three positions a saline tank (FIGS. 3D, 3E, and 3F), with the background saline resistivity of 10 Ω·meter. The potato was placed in three positions of the tank; left, center and right. The frequency response of the resistivity of the potato is shown in FIG. 3C.

FIGS. 3D, 3E, and 3F show a heat map of the RSCSRAF for each of the three positions of the potato sample (left, center and right), clearly showing that the system can image saline as having a low percent difference in spatial resistivity between two frequencies and shows the ability to track movement of biological structures beneath the patch.

As a proof of principle, an experiment was performed to determine if the system could detect saline beneath a biological structure separated by ribs. The biological tissue was mimicked using cut potatoes, the ribs were mimicked using a plastic grid. The thickness of the potatoes and grid was approximately 1 inch. The image in FIG. 3G shows a heat map of spatial relative percent difference in resistivity between two frequencies, clearly showing that saline, a substance of low percent difference in resistivity can be detected beneath a biological structure which includes ribs.

In operation, when determining lung wetness, the array of electrodes is first placed on the subject's torso, and the electrodes are connected to a processing unit. As discussed in more detail below, in some variations, the electrodes may be preconnected to a control unit which may include a processing unit; the control unit may apply current and sense voltages. The control unit may be integrated on the patch.

The system (e.g., a control unit/controller) selects two electrodes at a time as a current pair. A small electrical current is then passed between each pair of electrodes. Voltages may be recorded by electrodes positioned between the drive pair; although it may be possible to use electrodes outside of the drive pair as well. In some variations, the current and voltage data may be transferred to a secondary processing unit. Thus the system may include circuitry (e.g., a first processing unit) that is configured to condition and/or enhance the received voltage. In some variations only a single processing unit is included, which may integrate the function of the electrode conditioning/driving and analysis of the current/voltage signals.

For example, FIG. 4 illustrates one variation of a system 400 including an array of electrodes 401. The array of electrodes may be part of a patch or fixed positioning system, as just described, including both drive and sensing electrodes. In this variation, the electrodes are connected to one or more first processors 403′, 403″, 403′″ that includes active circuitry for conditioning and handing the current-injecting/detected signals. In some variations this first processor is coupled to (or integrated with) the sensor/patch/array of electrodes. The first processor(s) may connect to a controller and/or processor 405. Also as mentioned, in some variations only a single first processor (including a multiplexor for selecting which electrode pairs act as drive and sense electrodes. The controller may be integral with additional processors, or it may be a separate element 407.

After the array of electrodes of known geometry (e.g., spacing, size and configuration of the electrodes, relative to each other) has been applied, the system may apply currents from the drive electrodes and detect voltages using the sensing electrodes between the drive electrodes. The applied and sensed voltages and currents, along with the known spacing of the electrodes may then be used to solve for the distribution of resistivities within the tissue beneath the electrodes.

For example, using an array of electrodes such as that shown in FIG. 3A voltages may be sensed, as currents are applied, from various combinations of drive electrodes. In some variations, it may be beneficial (as described in detail below) to repeat the process for multiple driving current frequencies.

As discussed above, the devices and systems described herein may be used to determine the spatial relationship of resistivities and properties of resistivities of sub-surfaces below an electrode array when the electrode array is applied to a human body of unknown geometry, in such a manner that is least affected by errors in geometry. The geometry refers to the size and shape of the human body and the electrical internal boundaries such as the skeleton and other internal structures. In some variations, this invention may be applied to determine the likelihood of edema from skin layer to approximately 2″ to 2.5″ below the electrode array, where such areas of interest such as the lung are found.

In one example of the devices, systems and methods as described herein, an electrode array is applied to the body in a region of interest. The purpose of the electrode array is to provide an electro-mechanical connection to the body with predefined electrode spacing. As mentioned, the electrode array may be made up of a backing material with a printed array of electrodes, with printed metallic traces from the connector(s) to the electrodes, with a conductive hydrogel placed over the electrodes and a dielectric to protect and electrically insulate the printed traces. In one example, the backing material is made of polyester with titanium nitride; the electrode may be made of an Ag/AgCl pad measuring approximately 2″×0.150″; the electrode array spacing may be, for example, 0.36″ with approximately 30 electrodes (e.g., 31 electrodes) in each array.

The system may include hardware, firmware and/or software including logic to do the following: drive current through any combination of electrodes (current drive logic); measure the complex drive current (current measure logic); and measure complex differential voltages between any combinations of electrodes (voltage measure logic). As mentioned above, the system may also include logic (hardware, software, firmware, etc.) to determine the distribution of apparent resistivities and/or derived values (such as RSCSRAF values).

The systems described herein may determine (from the applied currents and measured voltages) various data types, including particularly spatial estimates of resistivities within the volume of tissue beneath the array of electrodes, and/or relative percent differences between spatial resistivities at different (e.g., between a high and a low) frequencies.

Any of the systems described herein may be implemented in a computer having a processor. For example, a system may include a processor configured to rank and/or eliminate the tetrapolar arrays.

In general, the sensor material and dimensions may be designed so that each electrode in the combined array (i.e. sensor) makes reliable mechanical and electrical contact to the subject's skin, where the skin curvature varies between subjects and varies with subject's position and movement.

In some variations, the patch may be configured as a narrow and thin patch (sensor) that makes reliable mechanical and electrical contact to a human subject over a range of subject motion. A thin and narrow patch has been found to be resistant to buckling, and hence, provide good electrical contact. Excessive bucking in the patch can cause significant reduction in the spacing between the electrodes, thus changing the depth of investigation. A thin and narrow patch was also found to conform well to the curvature of the subject; reducing the stress, strain, tension on the hydrogel and reducing electrical impedance variability. An important finding in the development of the patch was that with the proper selection of hydrogel adhesive, the weight of the patch and it associated wire harness can be sustained on the subject's skin without the need of any additional adhesive.

As mentioned above, the support backing may comprise any appropriate material, including a polyester material and an anti-bacterial titanium oxide material (e.g., coating, etc.). Further, in some variations the patch is conformable to the contour of a subject's back and has a thickness of less than about 5 mils.

The patch length may be designed to satisfy the electrical resistivity array objectives with a constraint that the center of the patch, which has the deepest median depth of investigation, and typically should align to the region of interest. In embodiments where the lung is the region of interest, the center of the patch may be aligned with the lung, with the cranial edge of the patch extending to below the shoulder and the caudal edge of the patch extending to above the waist.

The surface dimensions of the electrodes are designed to source adequate current into the body between any two electrodes on the patch from a differential constant voltage source or differential constant current source. Adequate current may be considered the current necessary to achieve the desired signal to noise ratio in both current and voltage measurements, as measured in apparatus, for the expected conductivity of the human body. Thus, exemplary electrode dimension may be 0.15 inches by 2.00 inches.

The patch may be configured to be easily handled by the operator and positioned on the subject. A graphic layer could be printed on the patch to visually aid the operator to the proper orientation of the patch. Visual marking may include “towards head”, “towards foot”, “center”, as mentioned above. Hydrogel may be sandwiched between the electrodes and a plastic release liner. The release liner would protect the hydrogel in storage and would easily be peeled away to expose the subject side of the hydro gel prior to application in the clinic. Tabs may be placed on the patch to handle and position the patch without interfering with the exposed hydrogel. All patch materials including the hydrogel may be biocompatible.

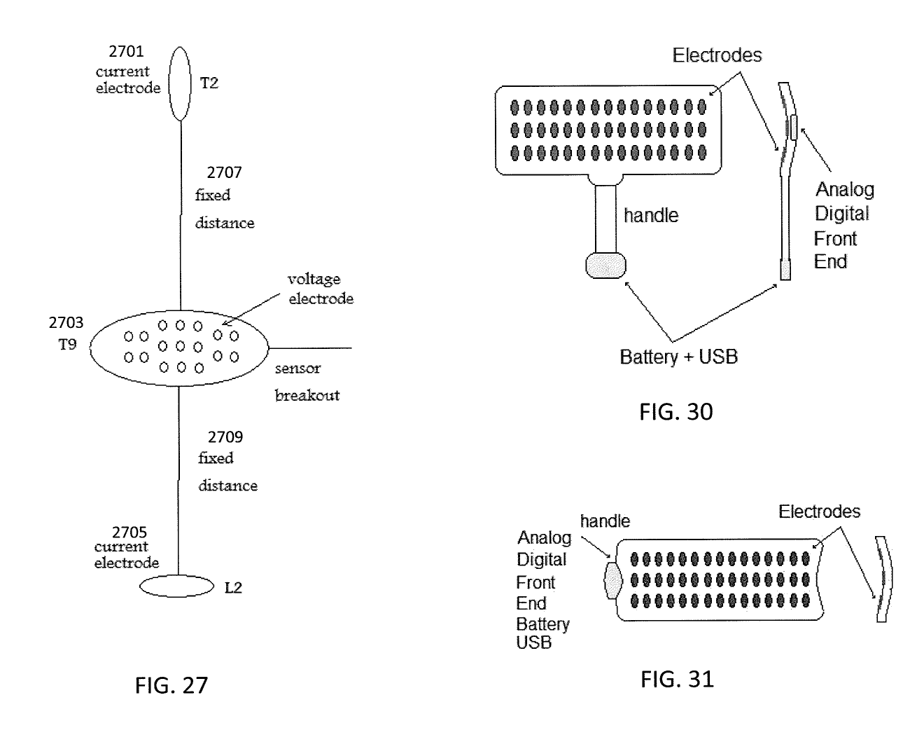

FIG. 27 shows another variation of a patch that may be used with the apparatuses described. In this variation, the patch has a central cluster of electrodes near the central region of the patch, as shown. These electrodes may be specifically configured as sensing (voltage sensing) electrodes. A connector 2707 may connect one end of the central region 2703 to a first drive electrode 2701 and a second connector 2709 may connect the other end of the central region to a second drive electrode 2705. The first and second connectors may be rigid or otherwise configured maintain a fixed distance relative to the central region and each other.

In the 2D array of sensor electrodes, each sensor electrode may be configured to make sufficient contact with the patient. For example, in some variations the sensor electrodes are approximately 0.2 inches in diameter or larger. The “pad” forming the central region may be, for example, 6 inches in diameter. The centrally located sensing electrodes may give a 2D grid, providing data for measurements at depth, in part because of the spacing provided by the first and second connectors. This configuration may also provide multiple redundant measurements, and, because the electrodes may be arranged in a grid, rather than just a line, it may also give more of the 3D structure, potentially providing information on “off-plane” regions (e.g., regions outside of the plane containing the two drive electrodes).

Using a Wenner-Schlumberger type array, the deepest sensitivity may be achieved when the drive electrodes are as far as possible from the voltage measurement pair. If the deeper tissue layers are of particular interest, the sensor can be simplified to two drive electrodes and a matrix of measurement electrodes, as shown in the example of FIG. 27. In another variation, a line of drive electrodes may be placed up and down the connectors in this design.

In FIG. 27, the top drive electrode 2701 may be shaped like an ellipse as to maximize its area while having maximum distance between itself and the scapula and spine, which may, for example, be placed vertically and centered between T1 and T2 on the right side of the subject's back. The bottom drive electrode 2705 may be placed horizontally around L2 (below the rib cage and above the hip bone). The matrix of measurement electrodes 2703 are near T9 (in the lower lung region) and clustered on a common (e.g., polyester) backing. As mentioned, two drive electrodes may be a fixed distance from the measurement electrodes by the connectors (e.g., via polyester backing material trace). In some variation, the connection to the controller may be made on the right (“sensor breakout”) and may connect to the voltage measurement matrix (not shown).

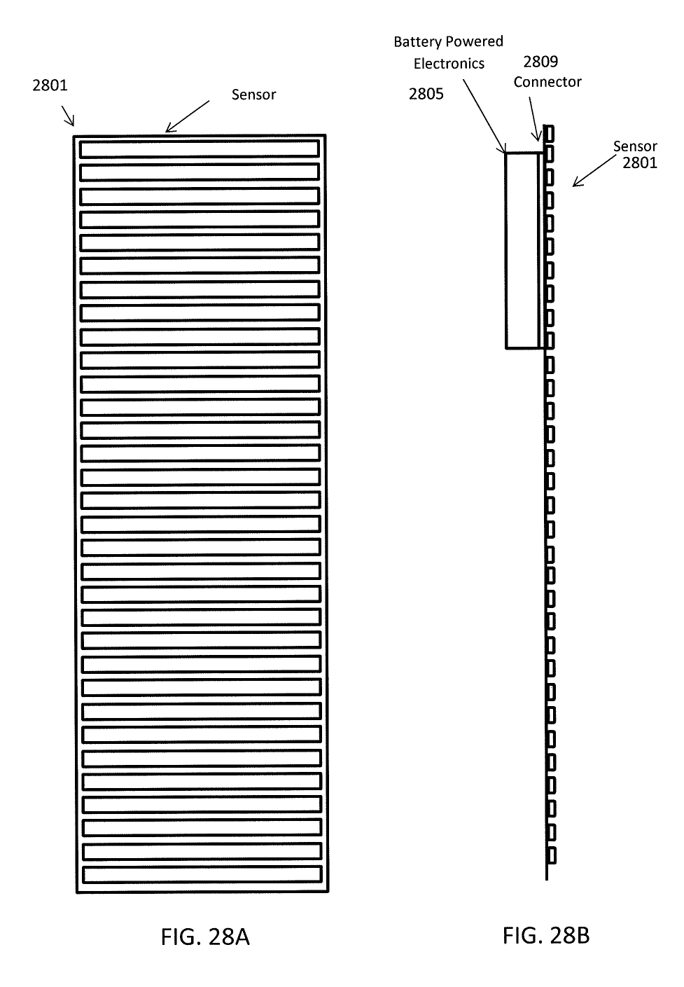

FIGS. 28A and 28B show another variation of a sensor, configured as a patch sensor with wearable electronics. As mentioned, the patch may be integrated with the circuitry (and/or power source) controlling the application of current and recording of voltages from the electrodes of the patch. In FIG. 28A, the patient-contacting surface of the patch 2801 may appear similar (or may be identical) to the patch configurations described above. FIG. 28B shows that controller electronics (“battery powered electronics”) 2803 are connected to the patch via a connector 2809.

In some variations, the sensor is configured as a disposable (e.g., single use) patch sensor. Alternatively, the sensor may be configured as a reusable (multiuse) patch sensor. For a single use patch sensor, the backing may be made of a polyester like material and the electrodes may be ink such as Ag/AgCl ink. For a multiuse patch sensor, the backing may be made of a flexible polymer such as neoprene, latex, or other similar material. This type of backing is typically cleanable are durable. The electrodes may be metal or carbon. A conductive substance such as a hydrogel can be applied in both disposable and reusable configurations. With single-use devices, the controller component (drive/read electronics 2805, similar to those shown in FIG. 25) may be miniaturized and may also be disposable, or may be reusable. For example, the connector 2809 may be configured to disconnect and reconnect to another disposable patch. As mentioned, this portion of the apparatus may include a battery, such as a Li-Ion battery. The on-board electronics 2805 may also include one or more sub-systems for optimizing the signals as described in part III, below. For example, this sub-system may include logic for determining which pairs of electrodes are best used as drive and/or sense electrodes, and may also include feedback to the patient or operator regarding the quality of the contact between the patient and the electrodes.

In general, variations in which the control electronics for controlling the application of current and recording of voltages offer numerous benefits, including ease of use, the elimination of long wires, improved measurement accuracy due to shorter connections, reduced EMI radiation and susceptibility due to shorted wires, easily protected against defibrillation shock, and the possibility of re-charging the battery on small base station. Data can be exported to host computer via wire or wireless connection, as discussed above.

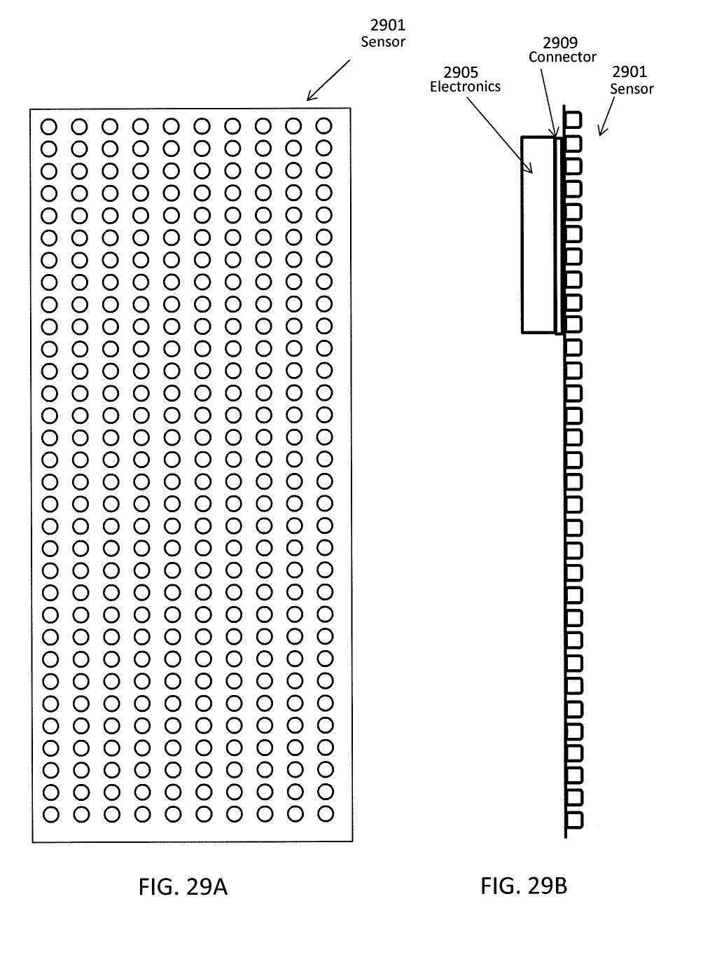

FIGS. 29A and 29B show another variation of a patch sensor configured as a 3D patch sensor 2901. In this variation, the patch includes an array of electrodes arranged as a grid across the patch that may result in a 3D detection of RSCSRAF beneath the patch and thus this may be referred to as a “3D” patch. As before, this patch may be single-use or reusable. For a single use 3D patch sensor the backing may be made of polyester like material and the electrodes made of ink such as Ag/AgCl ink. For a multiuse 3D patch sensor, the backing may be made of a flexible polymer such as neoprene, latex, or other similar material. This type of backing is cleanable durable. The electrodes could be metal or carbon. A conductive substance such as a hydrogel can be applied in both single-use and reusable variations.

As shown in FIG. 29B this variation of a sensor may also be configured to include on-board control electronics driving and reading from the electrodes, and in some variations, providing one or more outputs and/or determining tissue wetness (or passing the data used to determine tissue wetness to a second or additional processor).

In this configuration, an arbitrary number of columns and rows can be used to provide an actual 3D space map of the region beneath the patch. This may be used to look at features including alignment of the patch (e.g., confirming position over the lungs when determining lung wetness) and reducing the contribution of out-of-plane effects.

As described above for FIG. 26, a similar arrangement could be used with remote drive electrodes. Having more electrodes, and arranging the electrodes in a surface grid as suggested by this variation may provide higher resolution of subsurface details. Electrodes arranged in such a 2D array will allow for 3D modeling and capturing off-center-plane affects.

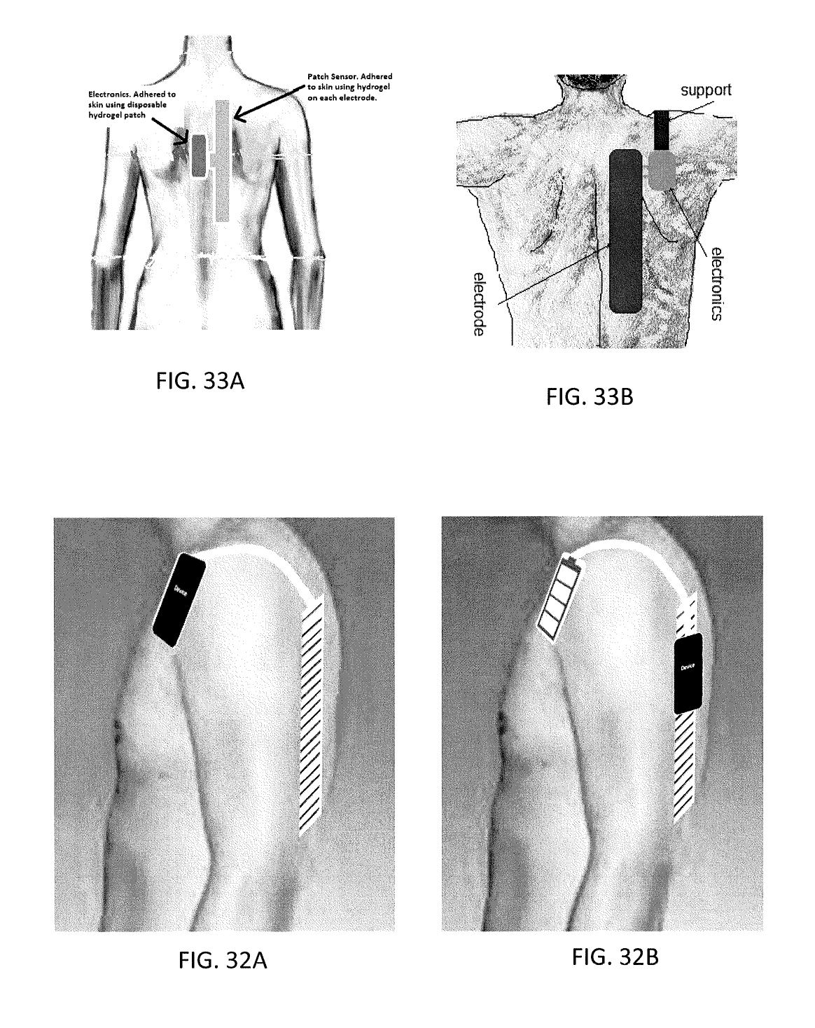

Other examples of sensors (patches) that may be used are shown in FIGS. 30-31, 38 and 39, as well as holders or supports for the sensor and other components of the apparatus, and particularly wearable systems, as shown in FIGS. 32-37.

For example, FIG. 30 shows a sensor configured as a paddle-like structure that can extend from the armpit to the ribcage. The electrodes are shown as arranged as a 2D electrode array, or could be a single line down the center region of the device (e.g., longitudinal electrodes arranged adjacently as described above). For the home-use market, the device may be used by the patient, or may be used by third party on a patient. Thus, the patient can themselves take the measurement, and may use the handle to hold and position the device. The control circuitry maybe mounted or integrated on or in the paddle structure.

The electrodes on the surface of the device could be used with or without a gel conductive. In some variations the patch electrodes portion of the device are disposable while the rest of the paddle is durable. For example, the patch may adhere to the paddle and can be removed from the paddle after taking measurements. In the some variations, the patch and electrodes are integrated into the housing of the device, and the entire thing is durable and reusable.

In use, the subject may hold the handle (which may house the electronics and/or battery). The subject may use one hand to hold it, and one hand to press it against the body, for example, the mid-auxiliary line on the side of the body. In some variations the device may be operated with one hand.

Any of these variations may be curved in one or more direction (e.g., saddle shaped). The curvature may match the curvature of the ribs. Many of these devices are for the patient's side, under the armpit (rather than the back). The device may be applied along the mid-auxiliary line. The device may be curved to match the curve of the mid-auxiliary region (e.g., concave around the side of the body).

In this variation, the electrodes are positioned on a paddle that is placed under the armpit. In one embodiment, the face of the paddle is curved as to make good contact with the subject's mid-axillary line. A handle allows the subject, for example, to hold the electrode paddle in place with his/her left arm, snuggly under the armpit, while using the right arm to press the electrodes against the mid-axillary line. In an embodiment of this design, the battery is place on the end of the handle to counterweight the paddle. In another embodiment, the paddle can also curve in-line with the mid-axillary line slightly as to fit snuggly against the subject's ribcage. A sketch of a paddle is provided below with a grid of electrodes; however another embodiment could have a single set of electrode in-line with the subject's mid-auxiliary line.

FIG. 31 illustrate another, similar variation. In this example, the apparatus is configured as a paddle that is adapted for being held by the user with one arm, so that a second arm is not needed to support the device. In the example shown in FIG. 31, the electrodes are positioned on a relatively stiff substrate (board) which fits snuggly against the subject's armpit (right-end of image below). In an embodiment of this design, the face of the paddle is curved as to make good contact with the subject's mid-axillary line. A handle allows the subject, for example, to hold the electrode board in place with his/her right arm, snuggly under the armpit, while using the same arm to press the electrodes against the mid-axillary line, thus leaving the left arm free (unlike the paddle version which may use both hands). The electronics and battery may be placed on the handle as to minimized the board's thickness and make it more comfortable. In another embodiment, the paddle can also curve along the mid-axillary line slightly as to fit snuggly against the subject's ribcage. A sketch of an electrode board is provided below with a grid of electrodes, but another embodiment could have a single set of electrode in-line with the subject's mid-auxiliary line.

The compact and wearable devices described herein may be supported on the patient's body by a support such as a strap and/or garment. In particular, the support may be an over-the-shoulder support. For example, an apparatus may be configured so that the various components of the apparatus (sensor electrodes, control circuitry, battery) are interconnected or interconnectable and may be held or supported on the same or different means, and may be separately arranged on the body.

For example, in some variations the sensor (including electrodes) is separated from the power supply and/or the control electrodes over the shoulder of the patient. FIG. 32A shows one variation of this. FIG. 32A shows the control circuitry (electronics) in a housing 3205 that is connected via a connector and cable 32011 to the sensor 3207 including electrodes. The patch (sensor) may be adhesively secured to the body, and connected to the control electronics which are worn over the patient's shoulder. The electronics may be tethered to the patch and supported over the shoulder by a cable, or by a strap, scarf, drape or other garment worn (e.g., over the subject's shoulder) by the subject. The power supply (e.g., battery) may be connected to the electronics and worn with the device, or it may be connected separately.

In FIG. 32B, the control circuitry is housed in an enclosure that his connected adjacent (or in some variations, on) the sensor (patch) and connected over the shoulder to the battery. Positioning the battery over the shoulder may provide a counterweight. In this version, the electronics connect to the patch via a small tab on the electrode. The battery which is heavy connects to the electronics using a long tail and rests on the front of the subject's pectoral. The weight of the battery offsets the weight of the electronics. As shown in FIG. 32A, the battery and electronics may be in the same package and both hang over the shoulder, to keep from pulling the patch off the subject's back. However, additional support can also be provided by using adhesive to support the weight of the electronics and/or battery. When the control circuitry (including data recording/transmitting) is separated from the patch, a long set of traces may connect the electronics to the patch and hence might be more susceptible to external noise sources. Those traces could be made more noise resilient by shielding the traces, where the additional impedance introduced by the shield is calibrated out.

In FIGS. 23A and 23B, the black box shown is the electronic “package” or enclosure (housing) containing the controller electronics. As mentioned, the electronics may be configured to include and act as a data acquisition/transfer component, allowing transfer of the information to an analysis unit (e.g., uploading, transmitting, etc.) that is separate, or the electronics may also include analysis and/or display capability. The battery may be the heaviest portion; as shown in FIG. 32B, the battery may be put over the shoulder, and may act as a counterweight, connected by a power cord/lead, which is unlikely to impact the noise or functioning of the device. The electrodes may generally have an adhesive and hydrogel so that it is adherent to the skin. The apparatus maybe applied to the subject by themselves or with the help of another person. In some variations the patch of electrodes is disposable; in some variations it's integrated with the housing and is reusable. There are many ways to hold the electronic package or battery against the patient, including a hydrogel/adhesive, or a Velcro material on the cable to prevent it from sliding on the body even with the weight of the electrode and battery. The battery itself could be secured to the patient (including an adhesive attachment, attachment to a garment, etc.). In some variations, one or more component is held in place by peel-off tape or adhesive (e.g., the battery component may be secured by adhesive to the patient).

In some variations, the enclosure for the electronics is secured to the patient adjacent to the patch (e.g., on the patient's back). As shown in FIG. 33A, an adhesive (e.g., 2-sided tape) could be used to secure the device to the skin. Alternatively, a hydrogel could be used to secure the electronics package to the patient.

The electronics enclosure may be attached close to the electrode patch as to minimize trace length and hence external signal interference. In this embodiment, the patch is aligned between the scapula and spine with the electronics package attached to the spine using hydrogel. However, the location of the electronics relative to the patch can be changed. The hydrogel may be used to secure both the electronics and patch. However, any two sided sticky adhesive may also work; a mild hydrogel can be used to avoid skin irritation.

As shown in FIG. 33B, in another embodiment, the electronic enclosure is attached to a sensor and a battery hanging over the shoulder to offset the weight. This prevents the patch (sensor) from becoming deformed by the weight of the electronics and/or battery. In some variations, the electronics enclosure (which may weigh a pound or less) could be secured to the patient's shoulder, as illustrated above. Again the housing for the electronics could be textured or otherwise include a non-slip surface to rest against the skin.

Any of the apparatus described herein may be configured to have reusable and/or disposable (single use) components. For example, described below are two versions of this concept; the first is based on a disposable patch that attaches to a semi-flexible material which houses the electronics, and a second version in which the electrodes are embedded into a semi-flexible backing material. In either configuration, the electrodes are configured in an in-line arrangement, as depicted below, or on a grid.

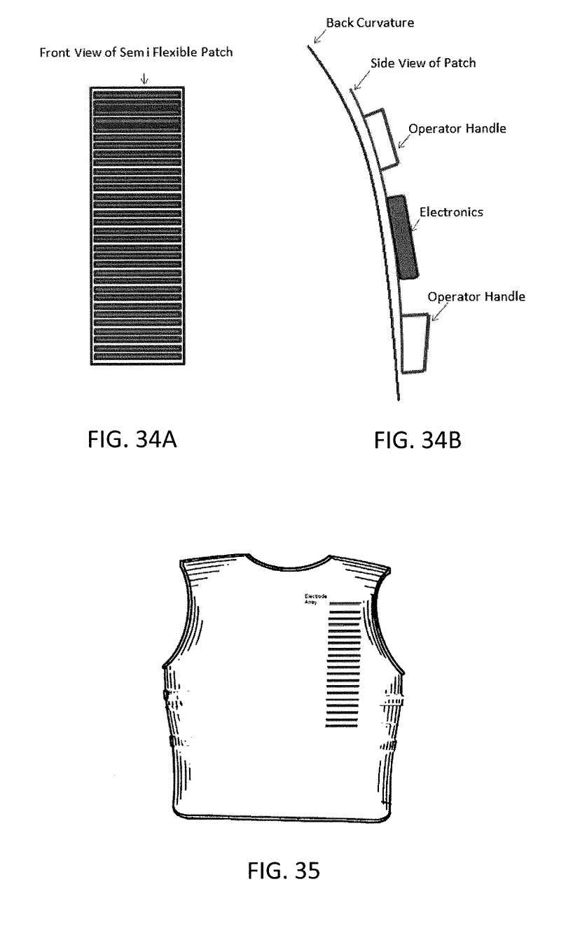

In the disposable patch version, the electrodes are attached to a semi-flexible backing (see, e.g., FIGS. 34A and 34B) using double sticky tape or some other similar adhesive material. The backing material provides a clip as to connect the electronics to the electrode patch. The two handles are used to push the electrodes against the subject's back. The backing material is flexible enough to conform to the subject's back.

In the non-disposable version, the electrodes are slightly offset from the semi-flexible backing material so each electrode can make good contact with the subject's back. In this version, the electrode can be made of stainless steel, or some other durable and electrically conductive material. The force required to press the electrodes onto the subject's back is provided by the operator via the two handles.

A screen, and/or indicators (e.g., LEDs, etc.) could be included, e.g., with the sensor and/or control electronics) that lets the operator know if the electrodes are making good contact before the test begins. An audible sound (tone, bells, voice, etc.) can alert the operator as to when the electrode make good contact and the test can begin. In another embodiment, the flexible packing could also house strain gauges to measure the curvature of the patch, thus knowing the distance between the electrodes and estimate the topography of the subject's back.

In some variations, an apparatus may include a garment that holds the electrodes and/or control electronics, power supply, etc., and can be worn to hold it in position. For example, a vest or jacket (including a compression fabric) may be used to push the electrodes against the skin to make sufficient contact. All of the electronics can be part of the garment. Additional materials or mechanisms could be used by the garment to push the electrodes against the back. For example, in some variations the vest includes an inflatable region that can drive the electrodes against the user's skin. Similarly, a foam memory material may be used.

FIG. 35 is one example of a garment (shown here as a vest) that includes an integrated patch (electrodes) or that houses the electrodes. The surface shown in FIG. 35 is the inner back portion of the vest, so that the electrodes can be held against the subject's skin. The vest may also hold or support the electronics and/or the batter or any other component of the apparatus. The vest can be secured to the subject, e.g., using Velcro straps, clips or ties. The vest can be inflated using internal bladders to make sure the electrodes makes adequate contact with the subject's back. The vest can be made of various materials both stretchy (like spandex or lycra) or taut like polyester. Impedance measurements are automatically taken whenever the vest clip closes (or a button on the front of the vest is pushed). In addition to a full vest, a bra like configuration can also be used to attach the electrode array to the subject's back, side or front, as shown in FIGS. 36A and 36B.

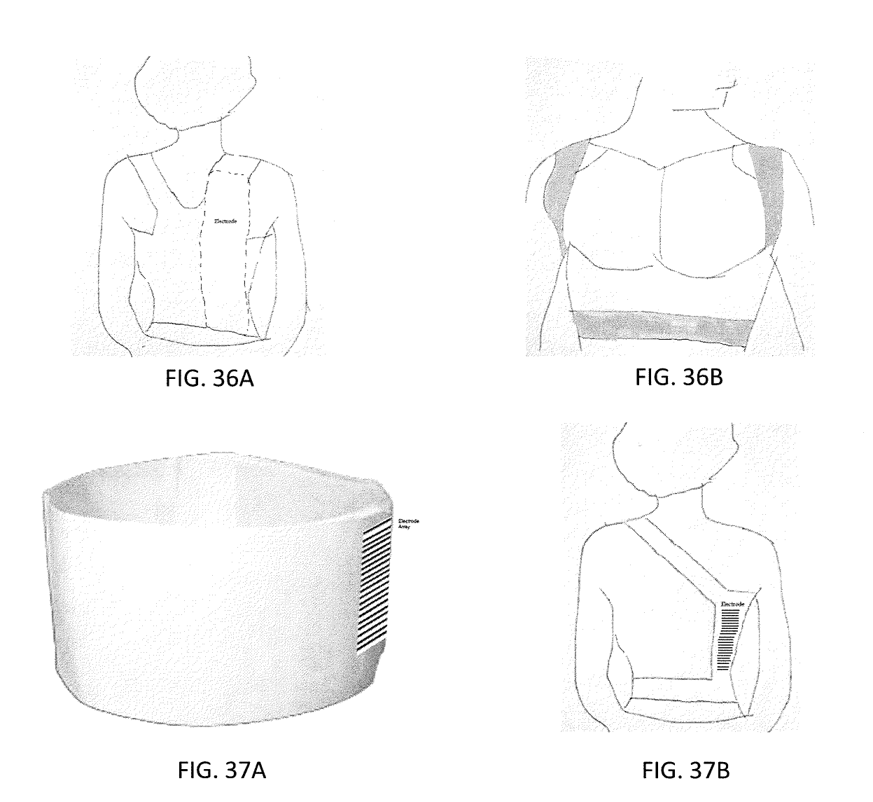

In some versions, as shown in FIGS. 37A and 37B, the patch connects to the subject's back and may sit in a strap or band to hold the sensor against the subject's body. A subject can then position this strap or band themselves using the strap or band to guide the patch placement. Once the patch is registered on the subject's back, the strap or band can be snugged (tightened) by adjusting the tension across the subject's front adjustment (via clip, Velcro, ties, etc.). As with the vest shown in FIG. 35, the pressure pushing the electrode onto the subject's back can be enhanced by adding foam between the strap and the electrodes, inflating an internal bladder, or some similar mechanism.

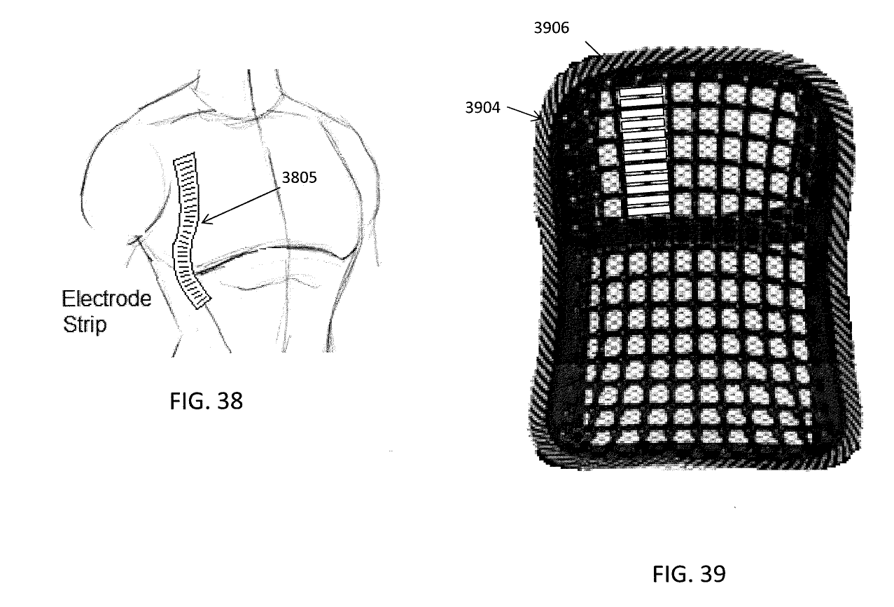

In some variations, the array of electrodes may be self-applied by the user to the front or front and side of the body. In this variation the strip may be curved, as shown below in FIG. 38. The electrodes may be positioned in the front and (in some variations) the side of the patient. To avoid non-target tissue (such as the breast, etc.) the electrode strip may bend or be pre-curved. Because the system may want to know the specific separation/spacing of the electrodes, the apparatus may include sensors (e.g., strain one or more strain gauge, as discussed below) to help deduce the curvature of the device and/or the more precise relative positions of the electrodes.

In general, the sensor and affiliated components described herein may be clipped onto to an electronics enclosure and/or battery and may be held by the patient or attached, as shown above, using an adhesive or other method.

A strip of electrodes may be placed in the front of the thorax. For example, FIG. 38 shows one example of a pre-curved sensor. The strip 3805 curves around the breast as to follow the anterior-axillary line. The curvature of the strip may change the distance between electrodes, so a strain gauge or similar apparatus or technique for determining curvature and positions of the electrodes may be used and/or integrated into the apparatus. In some variations, the in-line electrodes shown may instead be a 2D grid of electrodes.

Although the lightweight and compact systems described above describe wearable apparatuses (devices and systems) non-wearable apparatuses are also contemplated. For example, an electrode grid may be placed on the subject's back to measure lung hydration when the subject sits back on a chair. FIG. 39 shows one example of this, in which a chair 3904 includes an array (6×6) of electrodes that the subject can sit back onto to. The electrodes are not shown to scale. Other variations may include a mattress or pad that the subject may lie on and include the electrodes (including a linear array of electrodes as described above, e.g. in FIG. 28A). In 2D grids of electrodes, the grid could be different sizes and shapes to conform better to the subjects region of interest. For example, an array may be saddle-shaped so as to slightly push the electrodes into the subject back when the subject leans back on the chair example in FIG. 39

Some of the electrode variations described herein may produce electrode arrays which conform to the body by bending. To ascertain the amount of bending, and hence the distance between electrodes, strain gauges can be added to the patches as to measure the patch curvature during the data acquisition phase. As mentioned, this curvature information allows a model to account for body shape and electrode placement issues arising during testing. In addition, shallow electrode measurements (e.g., measurement of voltage from closer electrodes in the pair) may also be used to provide curvature information by solving for the boundary of the body required to produce measurements similar to those being acquired when the shallowest layer is assumed to be nearly homogenous.

Any of these variations may use more electrodes than otherwise necessary, so that some of them can be rejected or avoided, thus, extra measurements from “bad” electrodes can be rejected, as described herein. Further, many of the electrode sensor schemes detailed herein may provide only partial contact to some of the electrodes in the array. A grid type array may be beneficial when having to check the number of electrodes making good contact before starting measurement acquisition, as mentioned above. The subject may be made aware a “good” contact by a visual light or audible bell before the test begins. The pre-processing software may then flag those electrodes not making contact so that a suitable subset of electrodes can be selected to calculate the subject's hydration index.

### Part III: Optimizing or Enhancing the Signals for Determining RSCSRAF

Any of the apparatuses described herein may use one or more techniques to enhance the signals used to determine RSCSRAF. This may be done by reducing the system noise, including problems with the electrodes in the array. Signals may be filtered, or in some variations, averaged or removed from the sample set. For example, in some variations, only a subset of the “best” electrode combinations from the array of electrodes may be used to provide data to determine the RSCSRAF. Reducing the data sample size in this manner may be done prior to or after (or both) sampling the patient.

### Sensor Configuration and Electrical Resistivity Array Selection

The combined electrical resistivity array (the sensor), operates as a subject-applied portion of the device or apparatus for determining the spatial relationship of the relative spatial change in subsurface resistivity across frequencies in soft tissue beneath the surface of the sensor, which can be a interpreted to indicate tissue wetness. As described above, a sensor typically includes many electrodes that may be used in various subsets of drive electrodes and sensing electrodes to determine tissue wetness. In general, the more electrodes (m_{count}) in the sensor, the more combinations are possible.

In one example, the sensor may contain tens of fixed spaced electrodes, of which thousands of four-point electrical resistivity arrays can be configured. An electrical resistivity array includes two drive electrodes and two sensing electrodes. As described, the system or device typically determines the spatial relationship of the relative spatial change in resistivity in each cell of a mathematically determined, two-dimensional, multi-cell, cross-sectional grid, extending horizontally and vertically beneath the sensor. The grid may be sized to span a horizontal distance equal to that of sensor and may be sized in the vertical dimension to a specified depth of investigation (as defined by the combined electrical resistivity array). The relative spatial change in subsurface resistivities across frequencies may be determined for each cell in the grid by driving current and measuring voltage and using mathematical inversion methods to construct a spatial image of the relative percent differences in resistivity within the grid, as described above.

In an array of m_{count} electrodes thousands of four point electrical resistivity arrays are possible. Of the total possible number of electrical resistivity arrays, the system, devices and methods for determining tissue wetness typically uses a subset of said electrical resistivity arrays within the sensor. It is often desirable to include a very large number of possible electrodes (i.e., large m_{count}) to form the available pool of electrodes on the patch from which electrical resistivity arrays of drive/sensing electrodes may be chosen. However, not all electrical resistivity arrays provide equivalently sensitive/accurate signals for determining tissue wetness. The quality and sensitivity of a tissue wetness determination may be improved or optimized by selecting only those electrical resistivity arrays of electrodes from the available combinations in the sensor that would provide the most useful signals for determining tissue wetness. Described herein are systems, devices and methods for determining tissue wetness by selecting which subset of electrical resistivity arrays to use from an array of electrodes. In some variations this may be achieved by rating, grading, and/or scoring an electrical resistivity array and using only those that rate/grade/score sufficiently well to indicate that they would provide high quality signal information. The rate/grade/score (which may be referred to as a score, for convenience) may be compared to a threshold (e.g., a quality threshold) or range of acceptable values. In some variations the score is multidimensional, and may include multiple values. For example, the score for a particular electrical resistivity arrays may include a value (or values) for signal error (e.g., error due to placement, voltage error, current error, combined error, etc.), a value for depth of investigation (DOI), and a value for electrical resistivity array location. In some variations this score may be a combined (and/or weighted) value including one or more of these. As described in more detail below, the signal error for a particular electrical resistivity array may include more than one value (for placement error, voltage error, current error), or a combined (and/or weighted) single value.

A score within the desired threshold range (and/or above, or in some cases below a threshold) indicates that the electrical resistivity arrays should be selected. Conversely, a score could be compared to a rejection threshold/threshold range indicating that the electrical resistivity arrays should not be used. The devices and systems described may rate/grade/score individual electrical resistivity arrays of electrodes, and then use only those electrical resistivity arrays that score above a quality threshold for the tissue wetness determination.

As mentioned, an electrical resistivity array of electrodes is a subset of the total pool of electrodes and typically includes a pair of drive electrodes and a pair of sensing electrodes. Any size and configuration of electrodes forming the electrical resistivity array may be chosen. For example, an electrical resistivity array may include a pair of sensing electrodes between a pair of drive electrodes. More than two sensing and/or driving electrodes may be used.

For example, a sensor for use in determining lung wetness may support a combination of many four point electrical resistivity arrays of which many have a median depth of investigation necessary to reach the lung region in the human body when the sensor is applied either to the subject's back between the spine and scapula or applied to the subject's side along the mid-axillary line.

In operation, the system or device may grade all or a number of possible electrical resistivity arrays from the sensor and then choose which electrical resistivity array to use. In some variations the subset of electrical resistivity arrays are selected from the array after placing the sensor on the subject. The electrical resistivity arrays may be selected before applying current/sensing voltages for determining tissue wetness. In some variations the scores may be ranked so that the electrical resistivity arrays that are likely to provide the highest quality signal may be chosen. Alternatively, in some variations the electrical resistivity arrays may be chosen on the fly, so that the score of an electrical resistivity array is determined just before using it; if it falls within the acceptable range (e.g., above/below the quality threshold) a measurement (or measurements) are taken before selecting the next electrical resistivity array to examine. The score may be stored with the results from that electrical resistivity array, for later analysis or consideration.

Thus, in general, for any given sensor of array size m_{count}, out of all of the possible electrical resistivity arrays on the sensor, the systems and devices described herein may select electrical resistivity arrays based on their error, location, and/or depth of investigation (DOI). Although in general all three of these criterion may be used (error, location, and DOI), in some variations only one or two these factors may be used for selection.

In general the sources of error can be attributed to the three right-hand terms in the equation below, i.e., k, ΔV and I. Each source of error has a threshold in which it cannot exceed if we were to select it. Each electrical resistivity array measures one apparent resistivity. For example, where ΔV is the voltage measured across P1 and P2, I is the current measured on C1 or C2 and k is the “geometric factor”, a value that is derived by the electrode geometry, boundary of the body and spatial relationship between electrodes. As discussed above:

$${ρ}_{α} = k \frac{Δ V}{I} .$$

Errors may occur on the k, ΔV, and I.

For example, an error on k may occur by “mislaying” an electrode, since k is derived from the spacing of the four electrodes in relationship to each other. This means that although there are fixed distances between electrodes on the patch, the patch may be on a curved portion of the body (e.g., the back) or the subject's skin may be slightly wrinkled, and therefore the spacing between electrodes may not be as expected when applied to the subject. This may happen to some extent on any subject, so the systems, devices and methods may include a criteria to verify the k is fairly robust for small changes in spacing of the electrodes within an electrical resistivity array. To find out which electrical resistivity arrays have robust k, a “wiggle” test may be performed mathematically by varying the spacing of the four electrodes in relationship to each other by a one half electrode spacing in the calculations. If the calculated k values fall in a small range of values, it is considered robust; otherwise this electrical resistivity array may be rejected as being unstable or prone to changes with movement. The relative robustness of the electrical resistivity array may be provided with a numeric value that may be used in scoring the electrical resistivity array.

The range of k may be defined as

$$\vert \frac{δ k}{k} \vert .$$

An example of a wiggle test is provided below.

For any given electrical resistivity array, error may also be present in the voltage measurement, for example a typical voltage measurement may have a 1% to 4% error. Further, in some variations of the systems and devices described herein, a “noise floor” for measuring voltage is any voltage less than about 3 mV. Thus, electrical resistivity arrays may be chosen in which the predicted voltage is above this floor (e.g., 3 mV). In an exemplary sensor there may be many (e.g., thousands of) electrical resistivity arrays and a non-negligible percentage of these may have a predicted voltage less than 3 mV. In the equations below, we represent the error in voltage as

$$\vert \frac{δ U}{U} \vert .$$

An error may also be present in the current measurement. The term for current error in the equation below is |δI/I|.

The sign of the errors can be both positive and negative, so total allowable error is expressed as absolute value:

$$\vert \frac{{δ}_{{ρ}_{α}}}{{ρ}_{α}} \vert \leq \vert \frac{δ k}{k} \vert + \vert \frac{δ U}{U} \vert + \vert \frac{δ I}{I} \vert .$$

A threshold range may be provided based this error calculation. The threshold may be determined for each component (e.g., each type of error may be limited to be less than a threshold value, e.g., 5% absolute value for each), or the total error may be used. For example, if the three sources of error total to greater than 15% the electrical resistivity arrays may be rejected. The error criterion applied to determine a rank, score or grade for a particular electrical resistivity array may include each of these three categories of error, or just one or two of them. As mentioned, a threshold or weighting of these sources of error may be applied to each electrical resistivity array.

In addition to error, the location of an electrical resistivity array on the sensor and the median depth of investigation (DOI) may also be considered in determining if a particular electrical resistivity array may also be used.

Determination of the DOI is discussed and illustrated below, however, in general, electrical resistivity arrays in which the location and DOI are close to each other may be excluded, as using adjacent electrical resistivity arrays can confound the solution of the inverse problem by providing too much similar information. Thus, it may be desirable to space the shallow and mid-level electrical resistivity arrays out, but include all deep electrical resistivity arrays which typically are not as close to each other.

In one example, 252 Wenner-Schlumberger (W-S) electrical resistivity array were examined, and are listed in table 2 (FIG. 23), below. This table shows electrical resistivity arrays and lists (in the left column) the type of electrical resistivity array, as well as numbers indicating which of the electrodes correspond to C2, C2, P1 and P2. The right column indicates the calculated median depth of investigation (DOI). All of the electrical resistivity arrays in this table have a relative percent depth variance of less than 3% and a line charge K-factor that does not vary from the point-charge K-factor by more than 3%.

### Median Depth of Investigation (DOI)

An electrical resistivity array may be a configuration of (typically) four electrodes used for measuring electric current and differential voltage. Common electrical resistivity arrays types include Wenner-Schlumberger, Dipole-Dipole and Gradient. Refer to FIG. 1B for illustrations of these types. Electrode electrical resistivity arrays have been used outside of the tissue wetness application described here to measure resistivity across both large and small distances, for example, ground water reservoir surveys in geophysics and wafer fabrication applications in semiconductor manufacturing use electrical resistivity arrays such as those shown in FIG. 1B. In FIG. 1B, the current is driven between C1 and C2 and voltage drop is measured across P1 and P2.

In some embodiments, a sensor contains between 28 and 32 electrodes (m_{count} is between 28 and 32) providing thousands of combinations of four point electrical resistivity arrays. Each electrical resistivity array has a sensitivity pattern, where sensitivity in this context describes the degree to which a change in the resistivity in an area beneath the electrical resistivity array will influence the voltage measured between the sensing electrodes (P1 and P2). The cumulative sensitivity of multiple electrical resistivity arrays within the sensor produces the cumulative spatial sensitivity to the subsurface.

When the sensor is used as a subject-applied portion of an apparatus or system to detect the degree of wetness of the tissue, the sensor may be designed to maximize the number of electrical resistivity arrays that have a median depth of investigation (DOI) capable of penetrating to the depth of the tissue to be examined for hydration, but still provide robust responses. For example, with lung wetness, the sensor may be configured to have a DOI of roughly two inches, while still constraining the sensor so that the DOI is stable to small changes in electrode spacing and the measured signals should have a significantly high signal to noise ratio (e.g., noise is less than 5% of signal). Thus, the sensor should be configured so that there are sufficient “shallow” (e.g., closely spaced) electrical resistivity arrays to provided good coverage of the tissue around the region of interest.

The DOI can be determined for a four-point electrical resistivity array by first considering a single pole-pole array, as shown in FIG. 21. The change in potential, φ, measured on P1 caused by a change in resistivity, δp, in a small volume below the surface located at (x,y,z) is determined by the following mathematical relationship (M. Loke and R. Barker, “Least-Squares Deconvolution of Apparent Resistivity Psuedosections,” Goephysics, 60, pg. 1682-1690, (1995)):

$$δφ = \frac{δρ}{{ρ}^{2}} \int \int \int ∇ φ \cdot ∇ {φ}^{′} d τ .$$

Making use of this single pole-pole array, for simplicity, the potential, φ, generated by the current source of magnitude, I, at C1 across a homogenous half-space is

$$φ = \frac{I ρ}{2 π \sqrt{{x}^{2} + {y}^{2} + {z}^{2}}} ,$$

and similarly by treating P1 as a current source at some distance, a, its corresponding potential is

$${φ}^{′} = \frac{I ρ}{2 π \sqrt{{( x - a )}^{2} + {y}^{2} + {z}^{2}}} .$$

Taking the gradient of φ and φ′, the sensitivity function can be found explicitly in three dimensions as

$$\frac{δ φ}{δ ρ} = \frac{I}{4 {π}^{2}} \int \int \int \frac{{x ( x - a )}^{2} + {y}^{2} + {z}^{2}}{{( {x}^{2} + {y}^{2} + {z}^{2} )}^{3 / 2} {( {( x - a )}^{2} + {y}^{2} + {z}^{2} )}^{3 / 2}} d x d y d z .$$

The term inside the integral is known as the Frechet derivative,

$${F}_{3 D} ( x , y , z ) = \frac{I}{4 {π}^{2}} \frac{{x ( x - a )}^{2} + {y}^{2} + {z}^{2}}{{( {x}^{2} + {y}^{2} + {z}^{2} )}^{3 / 2} {( {( x - a )}^{2} + {y}^{2} + {z}^{2} )}^{3 / 2}} ,$$

and it defines the sensitivity for a pole-pole array (i.e., having a single drive electrode, C1, some distance, a, from the listening electrode, P1). Both electrodes are located on the x-y plane, where C1 is at the origin and P1 is displaced along x-direction a distance, a, the depth into the subsurface is given in terms of the z-coordinate.

The Frechect derivative provides a measure of the sensitivity in three dimensions, however, to estimate the depth of investigation confined to the z-direction, F_{3D}, is integrated along the x and y directions. The resulting integral has a simple analytical form (A. Roy and A. Apparao, “Depth of Investigation in Direct Current Methods,” Geophysics, 36, pg. 943-959, (1971)):

$${F}_{1 D} ( z ) = \frac{2}{π} \frac{z}{{( {a}^{2} + 4 {z}^{2} )}^{3 / 2}} .$$

Integrating the above equation from zero to infinity gives the total sensitivity value of a pole-pole array along the z-direction,

$${S}_{pole} = {\int }_{0}^{+ ∞} \frac{2}{π} \frac{z}{{( {a}_{C 1 P 1}^{2} + 4 {z}^{2} )}^{3 / 2}} d z = \frac{1}{2 π {a}_{C 1 P 1}} .$$

To obtain the sensitivity for a four-point (or tetra-polar) electrical resistivity array, the contributions from the four electrodes can be written as the sum of four pole-pole arrays,

$${S}_{array} = {\int }_{0}^{+ ∞} \frac{2}{π} \frac{z}{{( {a}_{C 1 P 1}^{2} + 4 {z}^{2} )}^{3 / 2}} d z - {\int }_{0}^{+ ∞} \frac{2}{π} \frac{z}{{( {a}_{C 1 P 2}^{2} + 4 {z}^{2} )}^{3 / 2}} d z - {\int }_{o}^{+ ∞} \frac{2}{π} \frac{z}{{( {a}_{C 2 P 1}^{2} + 4 {z}^{2} )}^{3 / 2}} d z + {\int }_{o}^{+ ∞} \frac{2}{π} \frac{z}{{( {a}_{C 2 P 2}^{2} + 4 {z}^{2} )}^{3 / 2}} d z ,$$

where a_{C1P1} is the distance between electrodes C1 and P1, and likewise for a_{C1P2}, a_{C2P1} and a_{C2P2}. However, the extent of the total sensitivity is infinite, what is needed is to find a finite depth at which the electrical resistivity array can sense a change in conductivity, otherwise known as the electrical resistivity array's depth of investigation.

A robust measure of depth of investigation is provided by the value at which the electrical resistivity array attains its median sensitivity value, i.e., where half of the sensitivity lies above and below this depth (Edwards L. S., 1977). It follows that the median depth of investigation, m, can be identified for any tetra-polar measurement by finding the upper limit, m, that satisfies the following equation:

$$\frac{{S}_{array}}{2} = {\int }_{0}^{m} \frac{2}{π} \frac{z}{{( {a}_{C 1 P 1}^{2} + 4 {z}^{2} )}^{3 / 2}} d z - {\int }_{0}^{m} \frac{2}{π} \frac{z}{{( {a}_{C 1 P 2}^{2} + 4 {z}^{2} )}^{3 / 2}} d z - {\int }_{0}^{m} \frac{2}{π} \frac{z}{{( {a}_{C 2 P 1}^{2} + 4 {z}^{2} )}^{3 / 2}} d z + {\int }_{0}^{m} \frac{2}{π} \frac{z}{{( {a}_{C 2 P 2}^{2} + 4 {z}^{2} )}^{3 / 2}} d z ,$$

and which simplifies to finding, m, for the following algebraic equation:

$$\frac{1}{2 {a}_{C 1 P 1}} - \frac{1}{2 {a}_{C 1 P 2}} - \frac{1}{2 {a}_{C 2 P 1}} + \frac{1}{2 {a}_{C 2 P 2}} = \frac{1}{\sqrt{4 {m}^{2} + {a}_{C 1 P 1}^{2}}} - \frac{1}{\sqrt{4 {m}^{2} + {a}_{C 1 P 2}^{2}}} - \frac{1}{\sqrt{4 {m}^{2} + {a}_{C 2 P 1}^{2}}} + \frac{1}{\sqrt{4 {m}^{2} + {a}_{C 2 P 2}^{2}}} .$$

Once the depth of investigation is known for a general tetra-polar electrical resistivity array, the sensor can be designed as to maximize the number of electrical resistivity arrays that have a median depth of investigation capable of penetrating to some desired depth.

For example, consider a dipole-dipole array whose electrode coordinates are C1=16, C2=18, P1=19, P2=21, and hence, a_{C1P1}=3, a_{C1P2}=5, a_{C2P1}=1 and a_{C2P2}=3. The depth of investigation as measured in electrode spaces is m≅0.507, and to calculate a DOI in inches, the distance between the electrodes (0.36″) is multiplied by m resulting approximately 0.18 inches. By carrying out the same procedure on a dipole-dipole array with coordinates C1=2, C2=4, P1=29, P2=31, its DOI is 2.42 inches.

The median depth of investigation must be stable to small changes in electrode spacing, in other words, the value of, m, should not change significantly when a_{C1P1}, a_{C1P2}, a_{C2P1} and a_{C2P2} are perturbed by some small amount. This exploration of stability can be done by prescribing some distribution for each term, u_{CiPj}, with a predefine deviation (e.g., ½ electrode spacing) and solving for the resulting deviation, Δm. Arrays that have small deviations as compared to the value, m, are considered stable (e.g., Δm/m<5%). Those arrays that exceed the specified deviation tolerance are not used.

To test the robustness of the placement (a “wiggle” test of error in k), consider a depth of investigation calculated above for the two dipole-dipole arrays, where m_{⊥}≃0.507 for the first and m₂≅6.736 for the second. For the sake of simplicity, we suppose the current drive electrodes remain stationary, but both listening electrode move together by ±½ electrode spacing (i.e., C1=16, C2=18, P1=19±½, P2=21±½ for the first and likewise for the second). The resulting change in the DOI are Δm₁≅[0.305, 0.677] and Δm₂≅[6.611, 6.862], and hence, their relative change are Δm₁/m₁≅73% and Δm₂/m₂≅4%. This result shows that the first dipole-dipole array's DOI is susceptible to a half-electrode deviation.

In this particular example the electrodes are close together in the first dipole-dipole array so a half-electrode deviation is larger as compared to the same deviation of a second dipole-dipole array. However, the size of the deviation is not necessarily proportional to the electrode spacing. For example, consider the following array, C1=2, C2=12, P1=7, P2=25, which has an m≅6.435 and its deviation is Δm≅[0.733, 3.177]. Note that while this array's DOI is similar in depth to the second stable dipole-dipole array and its electrodes are not close together, there is a nearly nine fold increase between smallest and largest depth of investigation measure. This example shows the important of verifying the robustness of the DOI measure to small changes in the electrode position likely to be experienced in the field.

In the previous example, electrical resistivity arrays were selected based on their depth of investigation and its robustness to small changes in electrode position. However, as the depth of investigation increases, the voltage drop measured between P1 and P2 becomes smaller, so it may be necessary to verify that the resulting voltage drop can be measured accurately before selecting that electrical resistivity array. Therefore, a signal to noise level threshold may be included or used in addition. The SNR threshold may also be used as a selection criterion to identify these electrical resistivity arrays that will or will not be used. This SNR threshold may be established by considering two mathematical models for the size of the voltage drop across P1 and P2. The first model is based on point current sources, the second on a line-charge model and both suppose the current is injected into a homogeneous half-space. The voltage value, φ, some distance, r, away from a point source with magnitude, I, decays as

$$φ = \frac{I ρ}{2 π r} ;$$

in a homogenous half-space with resistivity, ρ (Igel 2007, pg. 33-34). For the tetra-polar array, the voltage at P1 has the contribution for both the current sink at C1 some distance r_{C1P1} and the current source at C2 some distance r_{C2P1}. By superposition and I_{C1}=−I_{C2}, the voltage at P1 is

$${φ}_{P 1} = \frac{{I}_{C 1} ρ}{2 π r} + \frac{{I}_{C 2} ρ}{2 π r} = \frac{I ρ}{2 π} ( \frac{1}{{r}_{C 1 P 1}} - \frac{1}{{r}_{C 2 P 1}} ) .$$

A similarly expression provides the voltage φ_{P2} at P2 some distance r_{C1P2} and r_{C2P2} from C1 and C2, respectively. The voltage drop across P1 and P2 is given by

$$Δ {φ}_{P 1 P 2} = {φ}_{P 1} - {φ}_{P 2} = \frac{I ρ}{2 π} ( \frac{1}{{r}_{C 1 P 1}} - \frac{1}{{r}_{C 2 P 1}} - \frac{1}{{r}_{C 1 P 2}} + \frac{1}{{r}_{C 2 P 2}} ) .$$

This expression captures the size of the signal, Δφ_{P1P2}, given a tetra-polar point-electrode arrangement (i.e., C1, C2, P1, P2) and the product of the resistivity, ρ, of the homogenous medium and current, I. Moreover, note the connection between total sensitivity,

$${S}_{array} = \frac{1}{2 π {a}_{C 1 P 1}} - \frac{1}{2 π {a}_{C 2 P 1}} - \frac{1}{2 π {a}_{C 1 P 2}} + \frac{1}{2 π {a}_{C 2 P 2}} ,$$

and the voltage drop, Δφ_{P1P2}, where r_{CiPj} plays the role of a_{CiPj}. This implies that once the median depth of investigation is known, which uses the total sensitivity, the signal size is given by the product of the current, resistivity and total sensitivity (the reciprocal of total sensitivity is also known as the geometrical factor).

Using the same two dipole-dipole arrays (i.e., C1=16, C2=18, P1=19, P2=21 and C1=2, C2=4, P1=29, P2=31) and supposing the tetra-polar resistivity measurement was made using point-electrodes, the size of the voltage drops are

$$\frac{4 {l}_{2} ρ}{15 δ π} ≅ 0.928 V and \frac{4 {l}_{2} ρ}{19575 δ π} ≅ 0.7 mV$$

(i.e., supposing a 10 mA current, 10 Ωm resistivity and 0.0091 m electrode spacing). Thus, the voltage drop is approximately 1300 times smaller for the second dipole-dipole array, and a threshold must be used to guarantee the voltage signal is large enough to be accurately measured by the system. However, before establishing that threshold, the point-electrode model resulting in Δφ_{P1P2} will be expanded to an ellipsoidal electrode, as to predict the effects of the electrode's geometry on electrical resistivity arrays that are relatively close to each other.

As discussed above, electrodes cannot actually be points, as there has to be some dimension associated with the electrode and its area has to be suitably large to inject current into the body. Recall that FIG. 1C, discussed above, compares the voltage drop across P1 and P2 (ΔV) as measured by the instrumentation using rectangular electrodes (solid line) and the voltage drop predicted by the point-electrode model (dotted line). As is evident from FIG. 1C, when the electrodes are close to each other, the point-electrode model fails to correctly predict the voltage drop across P1-P2. The second ellipsoidal model was examined above in reference to FIG. 1D, showing that the second ellipsoidal electrode model achieves good agreement with experimental values.

For example, suppose that a measurement devise is capable of resolving 2 mV and a dipole-dipole arrays with an increasing gap between a fixed drive and listening electrode distances are used (refer to table 3). When the electrodes are very close (i.e., C1=2, C2=4, P1=5 and P2=7) the voltage drop is relatively a large 322 mV (assuming a 10 mA current, 10 Ωm resistivity and 0.0091 m electrode spacing), but when the listening electrodes are beyond electrode 23, the resulting voltage drop is too small to be measured accurately with a 2 mV resolution device, as illustrated in table 3:

TABLE 3

| C1 | C2 | P1 | P2 | ΔV |
|---|---|---|---|---|
| 2 | 4 | 5 | 7 | 322.2 |
| 2 | 4 | 7 | 9 | 86.3 |
| 2 | 4 | 9 | 11 | 35.2 |
| 2 | 4 | 11 | 13 | 17.5 |
| 2 | 4 | 13 | 15 | 9.8 |
| 2 | 4 | 15 | 17 | 6.1 |
| 2 | 4 | 17 | 19 | 4.0 |
| 2 | 4 | 19 | 21 | 2.8 |
| 2 | 4 | 21 | 23 | 2.0 |
| 2 | 4 | 23 | 25 | 1.5 |
| 2 | 4 | 25 | 27 | 1.1 |
| 2 | 4 | 27 | 29 | 0.9 |
| 2 | 4 | 29 | 31 | 0.7 |

Note that if we compare the size of the voltage drop of the line charge to that of the point electrode, the voltage size returned by both models agree when the spacing between the drive and listening electrode is sufficiently large. Recall that the point model returned a voltage drop of 0.7 mV (for C1=2, C2=4, P1=29, P31=7), which is equivalent to the line-charge model. However, when the electrodes are close to each other, the point electrode over estimates the voltage drop by nearly a factor of three (recall the point model returns 928 mV when C1=16, C2=18, P1=19, P2=21, which is a translated version of C1=2, C2=4, P1=5, P2=7).

### Electrical Resistivity Array Selection

There are various methods by which to select electrical resistivity arrays, for example in the previous three sections, the depth of investigation, in, its relative deviation, Δm/m, and its associated voltage size, ΔV, have been used to select arrays. Using a 31 electrode sensor in which the even electrodes drive current and the odd electrodes measure the voltage drop, there are

$$( \begin{matrix}16 \\ 2\end{matrix} ) \times ( \begin{matrix}15 \\ 2\end{matrix} ) = 12 \text{,} 600$$

possible combinations. Eliminating those combinations where both listening electrode are outside the drive electrodes, and ignoring the Gamma type arrays results in 5,460 arrays. Supposing the voltage drop should be larger than 5 mV and the relative deviation in the depth of investigation, Δm/m, less than 20%, there are some 2,500 arrays available (refer to FIG. 23).

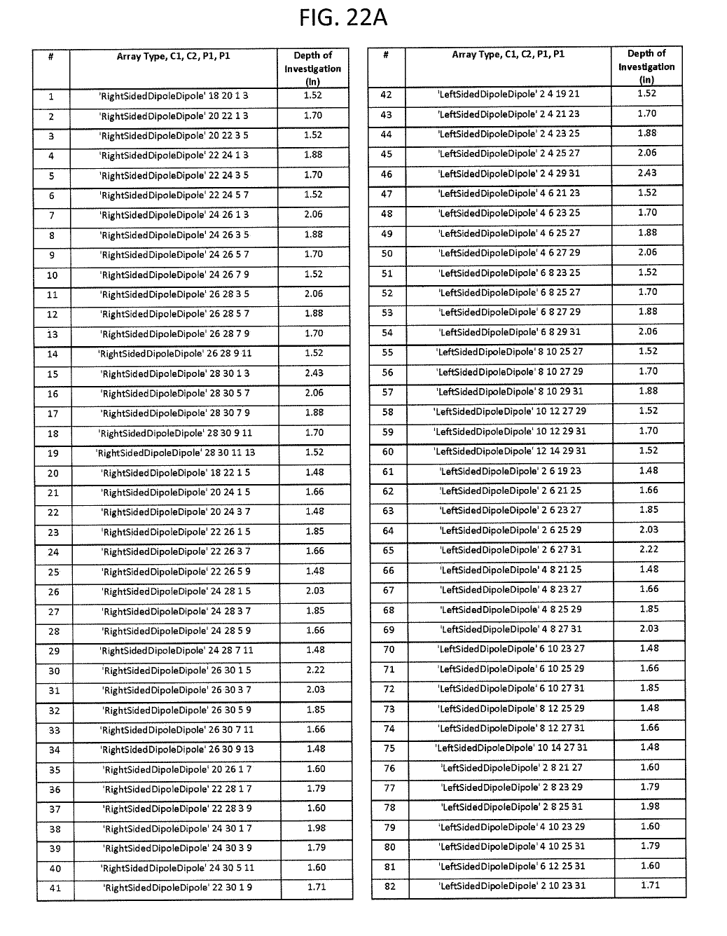

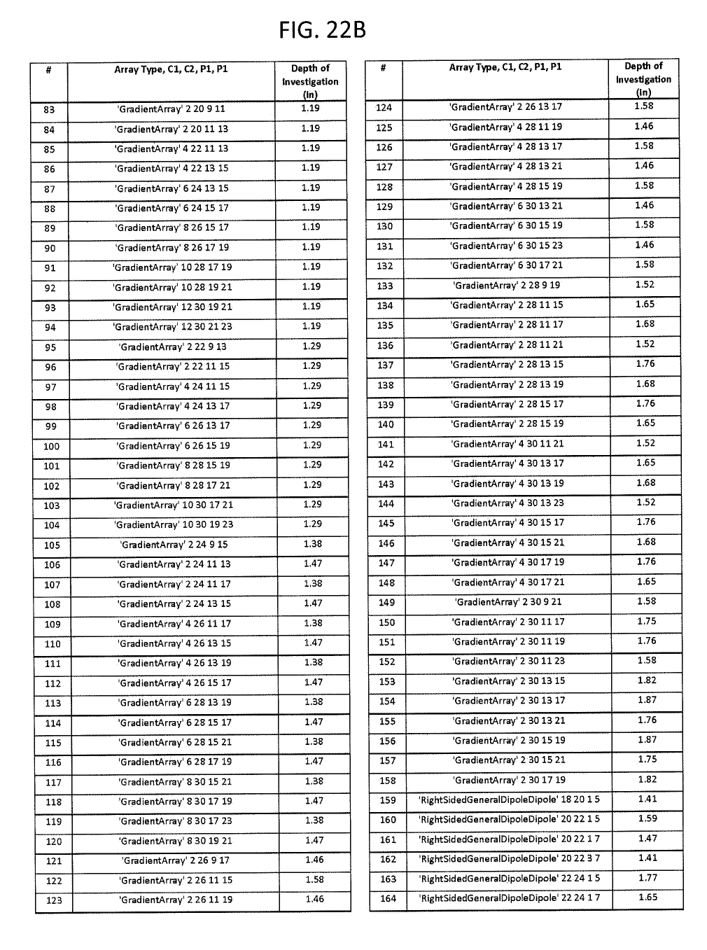

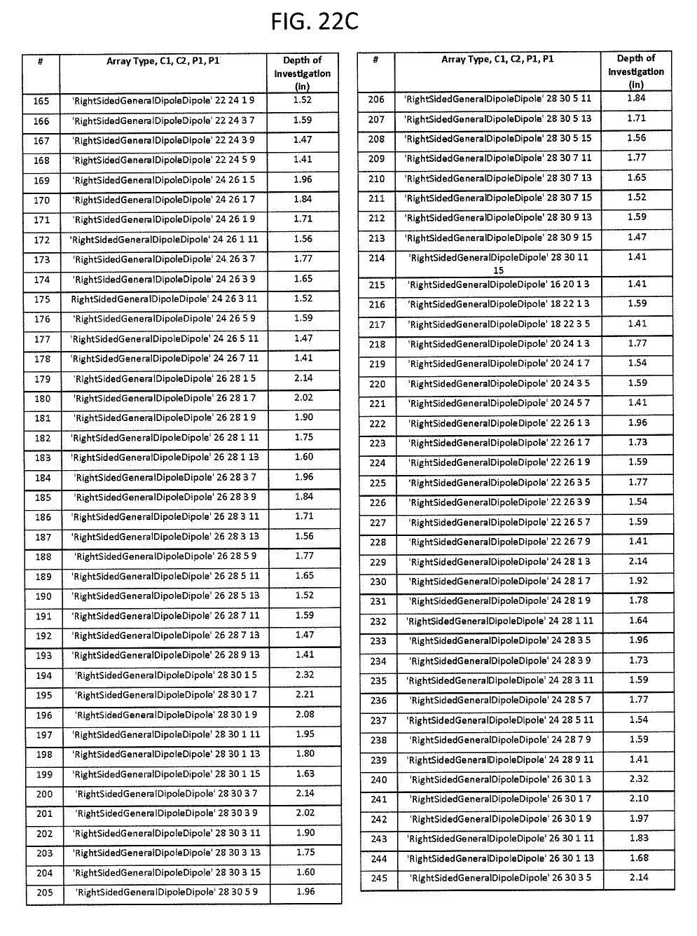

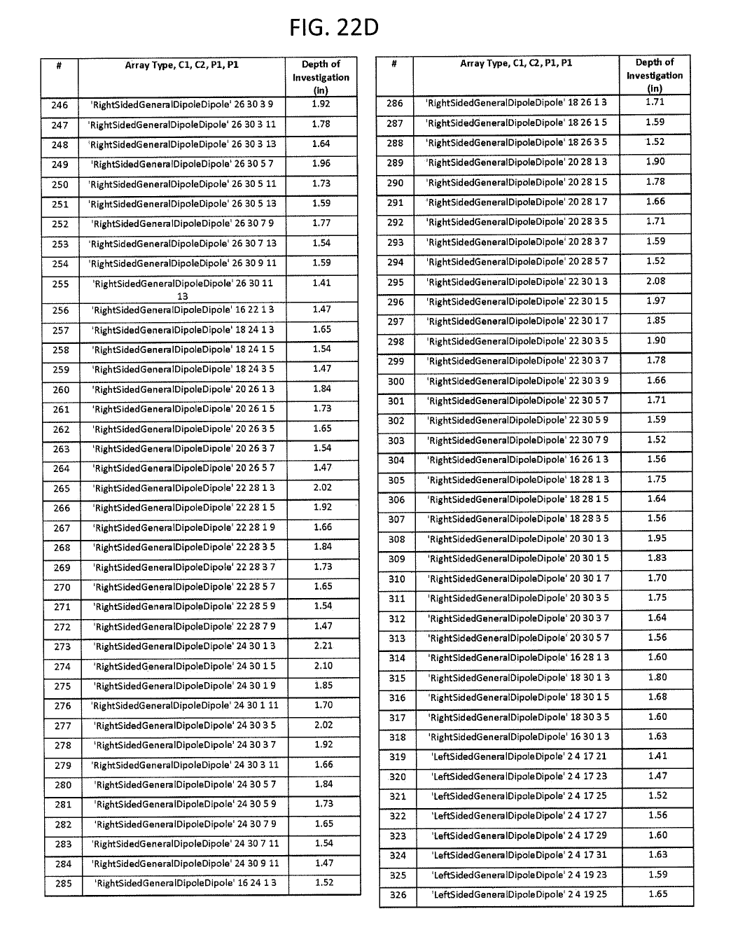

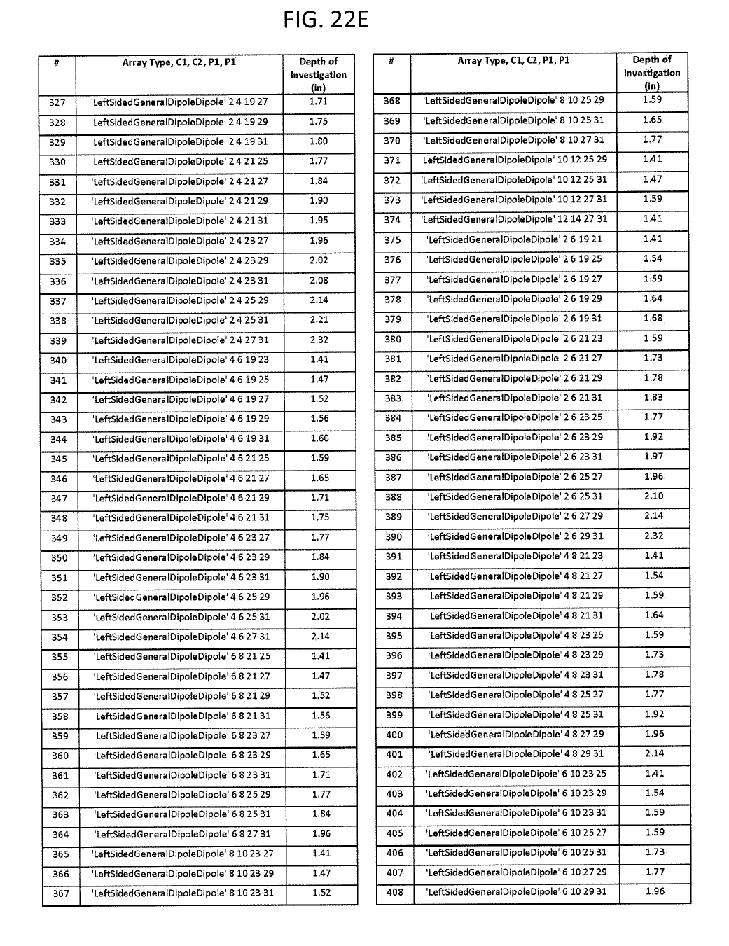

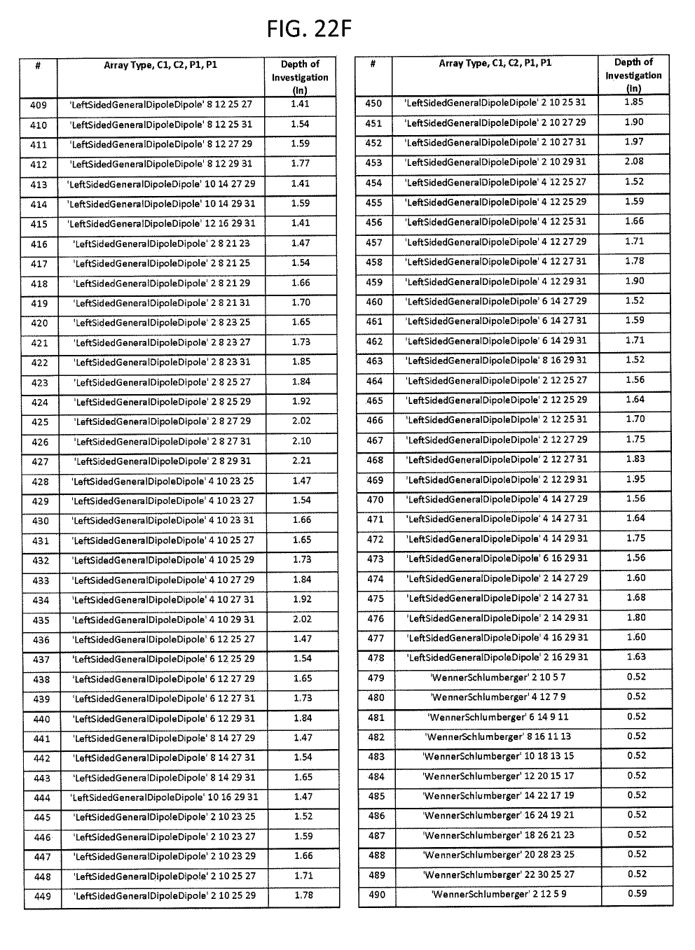

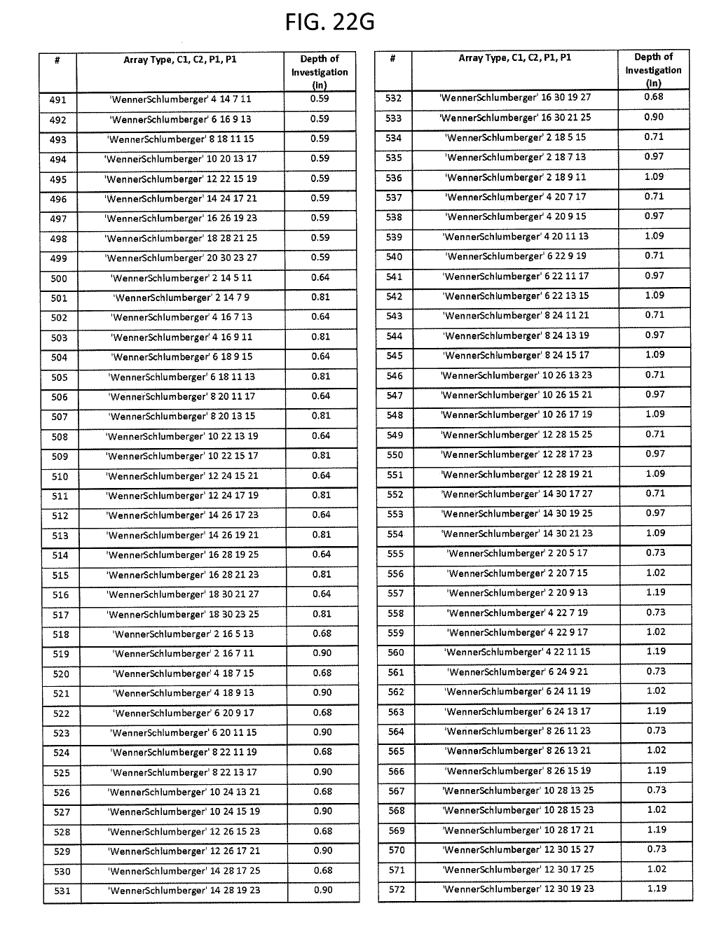

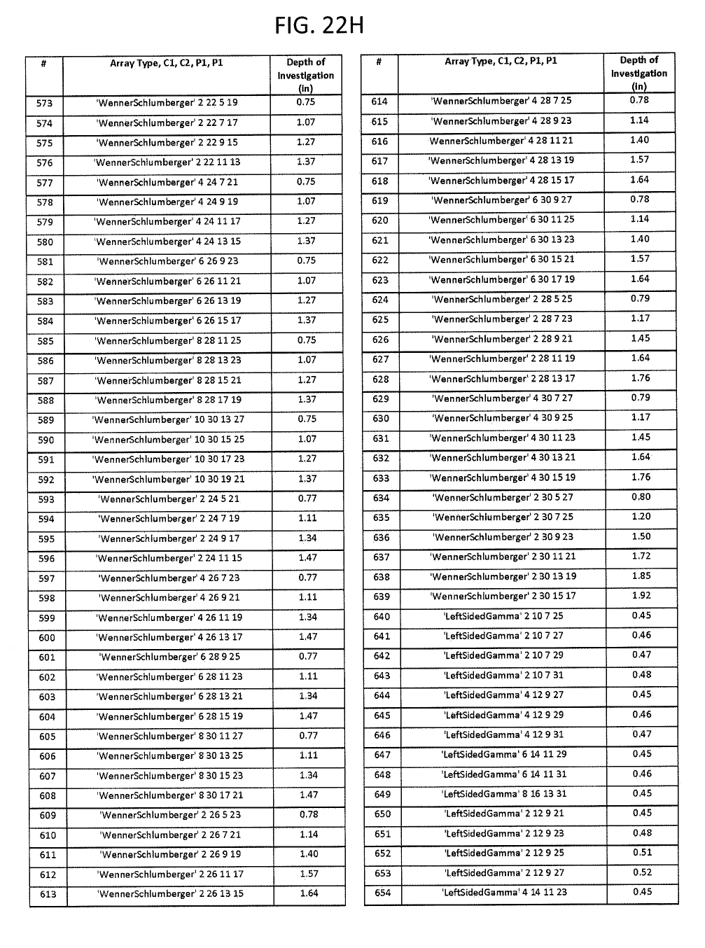

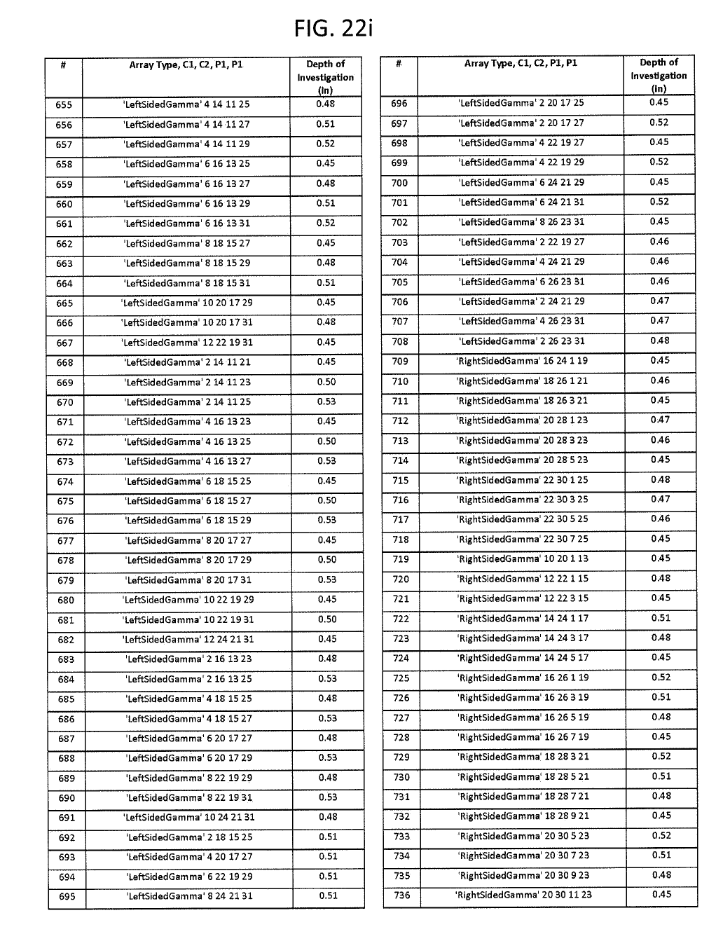

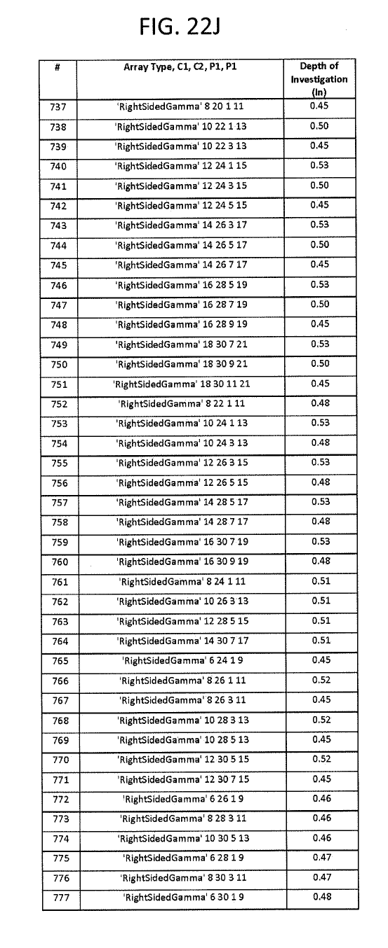

However, in some variations, it might also be of interest to leave the Gamma type arrays in the selection set and instead threshold based on the relative deviation between the point voltage drop and its line-charge counterpart, and limiting the DOI deviation to less than 3%. In this case 777 arrays are selected and are explicitly listed in the table (table 2) in FIGS. 22A-J. The selection criteria can also be electrical resistivity array type dependent and chosen to return a uniform coverage across the domain by taking into account the electrical resistivity array's DOIs and listening electrode locations. Therefore once the DOI, its deviation and associated voltage drop is known, electrical resistivity arrays can be selected appropriately.

### Exemplary Method of Discarding Electrical Resistivity Arrays Using Sensitivity and System Noise

The median depth of investigation (DOI) may serve as a measure of the amount of sensitivity a particular tetra-polar electrical resistivity array has to resolve a change in the subsurface resistivity at some depth. This may be calculated by assuming that the resistivity array sits on a surface of infinite extent and calculating the change in the field lines as generated by the drive and listening electrode pairs as if they were both driving current into the subsurface. The change in the field lines, as measured by the Frechet derivative, changes in three dimensions. To restrict this measure as to account for only the depth component of the sensitivity of a tetra-polar array, the Frechet derivative may be integrated across the surface plane, thus only the component that changes with depth remains. This depth of investigation (DOI) associates a sensitivity number to each tetra-polar array, which can be used to rank electrical resistivity arrays in terms of sensitivity. A “good” sensor may be considered one that has sufficient resistivity arrays with different sensitivities as to map the subsurface.

The accuracy of the associated sensitivities used to rank drive pairs may be examined and/or confirmed. For example a first method to verity the sensitivity ranking involves examining the depth of investigation to confirm that it is robust (e.g., doesn't change much) for small misplacements of electrodes, which could be due to the sensor wrinkling or bending. This may be accomplished by comparing the change in the depth of investigation due to electrode misalignment, with the size of this sensitivity measure (akin to a signal to noise ratio). If the change in depth over the depth value is larger than some predefined tolerance for a resistivity array, then that array is may be ranked low, and/or rejected for use in reconstructing the subsurface, as it was deemed unstable. A second ranking confirmation may be derived when the voltage sensing electrode pair is far from the current driving pair, as this makes the signal susceptible to electronic noise. This may be determined by making repeated measurements to find a noise floor of the measurement for the system and using this noise floor as a threshold for the smallest voltage measurement allowed to be considered when calculating an array's apparent resistivity. By method such as these, the arrays may be ranked; one or more of these methods may be applied. This ranking may be include multiple parameters (e.g., thresholds) and one or more of these parameters may be as a threshold for accepting or rejecting the array in the measurement. For example, arrays that report voltages below the noise floor, and/or arrays having a depth of investigation that changes significantly (e.g., more than x, where x is 2%, 5%, 10%, 15%, 20%, etc.) with electrode misplacement may be deemed unstable and eliminated.

Thus, given a noise floor, n, an electrical resistibility array may be deemed stable if its voltage measurement is greater than this noise floor (e.g., ΔV>n); alternatively or additionally, the array may be deemed stable if its depth of investigation, m, deviates by less than some threshold amount (e.g., Δm/m<5%), where Δm is the change of depth as a function of electrode misplacement (as an example, approximately ½ electrode width in each direction). The remaining resistivity arrays may be considered stable and can be ranked by their depth of investigation and used in the inversion software to find the subsurface measure of interest, as discussed above.

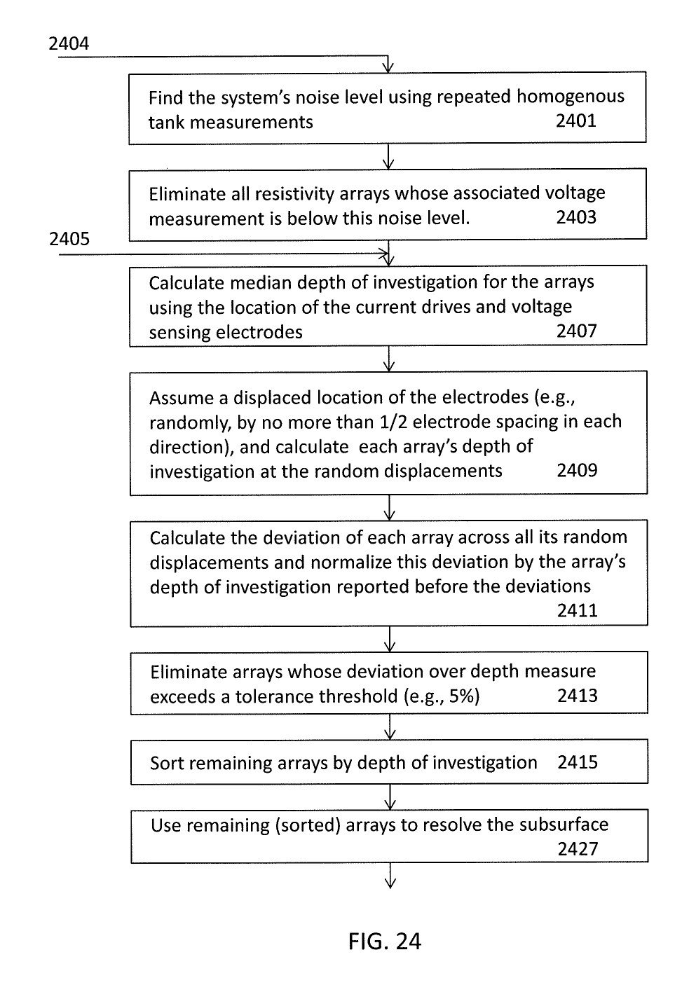

One variation of a method or system for determining which arrays to use from a sensor having a plurality of tetrapolar arrays is illustrated in FIG. 24. For example, in some variations, a system may be configured to first apply a noise level and classify arrays as above or below (or at) this noise level. For example, a system may first find the system's noise level using repeated homogenous tank measurements, as discussed herein 2401. Based on this noise level, the system may eliminate resistivity arrays whose associated voltage measurement is below this noise level 2403. Additionally 2404 or alternatively 2405 the system may calculate median depth of investigation for the arrays (or just for the remaining arrays after applying the noise level cutoff) using the location of the current drives and voltage sensing electrodes 2407. The system may then calculate, by assuming a displaced location of the electrodes (randomly, by no more than ½ electrode spacing in each direction), each array's depth of investigation at the random displacements 2409. The system can then calculate the deviation of each array across all its random displacements and normalize this deviation by the array's depth of investigation reported before the deviations 2411. Any array whose deviation over depth measure exceeds some tolerance (e.g., 5%) may then be eliminated 2413. In some variations, the remaining arrays may then be sorted in terms of their depth of investigation 2415. Finally, the remaining arrays may be used to resolve the subsurface 2415 as described herein.

### Data Filtering

Other techniques for improving or enhancing the determination of RSCSRAF may include filtering the data. For example, in some variations the apparatus may be set to provide a voltage “floor” for sensed voltages which operates as the noise floor. Thus, the apparatus may be configured to ignore or remove voltages below this noise floor threshold (e.g., 2 mV).

In some variations the apparatus may be configured to detect problematic measurement from electrodes or groups of electrodes. Various criteria may be used to detect problematic measurements. First, the apparatus may be configured to ignore all voltage values below lower bound or floor (e.g., 2 mV) as mentioned above. In variations using non-constant current sources, the apparatus may be configured to ignore all measurements below lower bound, e.g., 0.5 mA.

In some variations the apparatus may be configured to check end-to-end impedance bounds across each frequency and flag outliers, e.g., 100Ω<Z<500Ω at 200 kHz. The system may be configured to determine “reasonable” impedance bounds based on either predetermined values or set by experimental data, including based on skin contact. This technique may be used to determine if one or more driving electrodes are bad. If the current can't be maintained well, this will be apparent with this set of parameters.

In some variations, the apparatus may bound the measurement variability using the ratio of the voltage standard deviation to its mean. For example, the apparatus may flag measurements if the std/mean>5%. In any of these variations flagged measurements may be rejected or may be modified (e.g., filtered, weighted, etc.) in some way. This technique of determining if the standard deviation divided by the mean exceeds some threshold is also described as the variational coefficient; if for a particular measurement, the value changes compared to size of measurement, the apparatus may flag (e.g., reject) it. This effect may also indicate that there is not good contact, or there is too much movement by patient. Thus, the electrode (rather than just the measurement) may be flagged. Electrodes that give large variations (based on a predetermined threshold) during testing may be left out of the stimulation. If enough electrodes are flagged, the system may alert the user or patient that there is not adequate contact and to re-apply the patch.

The apparatus may be configured to check the sign of the pseudo-impedance determined from a set of data. Although the determined impedance can be negative, non-positive values are likely to be due to signal noise. The pseudo-impedance is the size of the change of voltage over the change in the current. Regions in the body may have a negative impedance because of noise rather than because of the measurement, thus a change in the sign may indicate noise in the measurements.

As already discussed, another check or filter on the data signal quality is reciprocity. Thus, in some variations the apparatus is configured to determine if the signal from the sense electrodes is equal or substantially the same as the signal from the drive electrodes after using the sense electrode to drive the dive current. Thus, the system may flag the electrodes if the reciprocal measurements are not identical or near identical, and flag the measurement if the difference between the reciprocal measurements is more than 10% different.

In some variations, the apparatus may be configured to identify measurements that vary over time more than some threshold level. For example, the apparatus may determine a boundary for the changes in the relative percent difference (RPD) based on data model to identify time series outliers. If the measurement changes dramatically over time, there is a greater chance that the measurement is a bad one.

In some variations, the apparatus may also determine bad (or inaccurate) measurements by comparing across neighboring electrodes in the array and flagging outliers. By comparing measurements made from neighboring electrode pairs, electrodes that should have approximately the same DOI (depth of investigation) can be compared; if their results are dramatically different, then there is likely to be an error. For example, when neighbors are physically close to each other and have nearly the same DOI, then if a trend of values diverges too much (“too much” may be determined by a preset threshold, such as a percent difference, of more than 10%, more than 15%, more than 20%, etc.) this may indicate a problem in one or more of the electrodes.

In any of these variations described above in which the apparatus determines that an electrode or measurement is likely to be “bad”, either the measurements or the entire electrode may be removed from the analysis. In some cases the system may also alert the user and/or patient to address the problem, for example by indicating that the user should reapply the patch.

Although various examples and illustrations are provided herein, these examples are not intended to be, nor are they, limiting. Other variations, including variations in the types, shapes and sizes of electrical resistivity arrays and individual electrodes, as well as the systems described herein, are contemplated. Further, although the majority of the examples discussed above describe the use of these devices, systems and methods to determine lung wetness, many of these devices, systems and methods may be used or adapted for use to determine the wetness of other body regions, not limited to lung. Thus, these methods, devices and systems may be used to treat disorders other than those associated with lung wetness (such as congestive heart failure). For example, the methods, devices and systems may be used or adapted for use to detect and monitor lymphedema, for use during hip replacement, or for monitoring, detecting or helping treat compartment syndrome. The claims that follow may set forth the scope of the invention described herein.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

It should be understood that features and sub-combinations of the inventions described herein may have utility alone or in combinations not explicitly described herein. Further the inventions described herein may be employed without reference to other features and subcombinations. Many possible embodiments may be made without departing from the scope, and it is to be understood that all of the subject matter herein set forth or shown in the accompanying drawings is to be interpreted as illustrative, and not limiting.

1. A compact and lightweight device for detecting tissue wetness, the device comprising:
a flexible sensor having a front and a back;
an array of electrodes arranged on the front of the sensor;
control circuitry configured to apply a constant current at a plurality of frequencies to the array of electrodes, the control circuitry comprising:
a constant current source,
a multiplexer adapted to select electrodes from the array of electrodes to act as a current source electrode, a current sink electrode, and pairs of voltage sensing electrodes,
a controller connected to the multiplexer and the constant current source and adapted to drive current between different combinations of current source and current sink electrodes and to record voltages from the sensing electrodes; and
a connector on the sensor adapted to connect the control circuitry to the array of electrodes.

2. A compact and lightweight device for detecting tissue wetness, the device comprising:
a sensor having a front and a back;
an array of electrodes arranged on the front of the sensor;
control circuitry configured to apply a constant current at a plurality of frequencies, the control circuitry comprising:
a constant current source comprising a wideband digital to analog converter,
a multiplexer adapted to select electrodes from the array of electrodes to act as a current source electrode, a current sink electrode, and pairs of voltage sensing electrodes, and
a controller connected to the multiplexer and the constant current source and adapted to control the multiplexer to sequentially drive current between different combinations of current source and current sink electrodes and to record sensed voltages; and
a connector on the sensor adapted to connect the control circuitry to the array of electrodes.

3. A compact and lightweight device for detecting tissue wetness, the device comprising:
a sensor having a front and a back;
an array of electrodes arranged on the front of the sensor;
control circuitry configured to apply a constant current at a plurality of frequencies, the control circuitry comprising:
a first constant current source;
a second constant current source that is 180 degrees out of phase with the first constant current source;
a multiplexer adapted to select electrodes from the array of electrodes to act as a current source electrode, a current sink electrode, and pairs of voltage sensing electrodes, and
a controller connected to the multiplexer and the first and second constant current sources, and adapted to control the multiplexer to sequentially drive current from the first constant current source on the current source electrode and current from the second constant current source on the current sink electrode and to record voltages sensed on the voltage sensing electrode pairs;
a connector on the sensor adapted to connect the control circuitry to the array of electrodes.

4. The device of claim 1, wherein the constant current source comprises a digital to analog converter configured as a bipolar, differential, voltage-controlled constant current source.

5. The device of claim 1, wherein the multiplexer is a crosspoint switch matrix.

6. The device of claim 1, further comprising an enclosure on the back of the sensor housing the control circuitry.

7. The device of claim 1, wherein the array of electrodes comprises more than 10 electrodes, wherein each electrode has a length that is greater than five times its width.

8. The device of claim 1, wherein the array of electrodes comprises a linear array of electrodes.

9. The device of claim 1, wherein the front of the sensor comprises a bio-compatible adhesive.

10. The device of claim 1, wherein the control circuitry comprises a second constant current source that is 180 degrees out of phase with the constant current source and wherein the controller is configured to drive current from the constant current source on the current source electrode and to drive current from the second constant current source on the current sink electrode.

11. The device of claim 1, further comprising a data recording unit configured to record the voltages and an indicator of the current source electrode, a current sink electrode, and voltage sensing electrodes from which the voltage was sensed.

12. The device of claim 1, further comprising a processor adapted to calculate a frequency response of an electrical parameter of the tissue beneath the sensor from the sensed voltages.

13. The device of claim 1, wherein the control circuitry is configured to apply a constant current at two or more frequencies between about 10 kHz and about 200 kHz.

14. The device of claim 1, wherein the device weights less than 2 pounds.

15. A method of determining tissue wetness based on the frequency response of an electrical parameter of a region of a tissue beneath a sensor, the method comprising:
applying a sensor having an array of electrodes on the subject;
sequentially repeating the steps of:
using a multiplexer to select, from the array of electrodes, a current source electrode, a current sink electrode, and pairs of voltage sensing electrodes,
applying a constant current at a plurality of different frequencies between the current source electrode and the current sink electrode and sensing voltages between the pairs of voltage sensing electrodes; and
calculating an electrical parameter of the tissue beneath the sensor from the sensed voltages at the applied frequencies; and
determining an indicator of tissue wetness from the electrical parameter.

16. The method of claim 15, wherein applying the constant current comprises applying an in-phase constant current to the current source electrode and applying a 180 degree out-of-phase constant current to the current sink electrode.

17. The method of claim 15, wherein applying the sensor comprises placing the sensor on the subject's back so that a long axis of the sensor is a proximal to distal axis that extends cranially to caudally along the subject's back, and wherein the electrodes on the sensor are positioned lateral to the subject's spine.

18. The method of claim 15, wherein determining the indicator of tissue wetness comprises determining an indicator of lung wetness.

19. The method of claim 15, wherein calculating the electrical parameter comprises calculating resistivities for region of the tissue beneath the sensor.

20. The method of claim 15, wherein determining the indicator of tissue wetness comprises determining the frequency response for the region of the tissue beneath the sensor.
